SEQUENCE LISTING (25 sequences)

SEQ ID NO: 1 - protein, 5 aa, synthetic construct
SFGMH

SEQ ID NO: 2 - protein, 17 aa, synthetic construct
YISSGSSTIYYGDTVKG

SEQ ID NO: 3 - protein, 15 aa, synthetic construct
EGGYYYGRSYYTMDY

SEQ ID NO: 4 - protein, 16 aa, synthetic construct
RSSQSIVHSNGNTYLE

SEQ ID NO: 5 - protein, 7 aa, synthetic construct
KVSNRFS

SEQ ID NO: 6 - protein, 9 aa, synthetic construct
FQGSHVPPT

SEQ ID NO: 7 - protein, 124 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 8 - protein, 112 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 9 - protein, 454 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 107 aa, synthetic construct [sequence omitted]

SEQ ID NO: 13 - protein, 42 aa, unidentified [sequence omitted]

SEQ ID NO: 14 - protein, 40 aa, unidentified
DAEFRHDSGYEVHHQKLVFFAEDVGSNKGAIIGLMVGGVV

SEQ ID NO: 15 - protein, 5 aa, synthetic construct
TYWIT

SEQ ID NO: 16 - protein, 17 aa, synthetic construct
DIYPGSSISNYNEKFKS

SEQ ID NO: 17 - protein, 10 aa, synthetic construct
EDGYDAWFAY

SEQ ID NO: 18 - protein, 16 aa, synthetic construct
RSSQSILHSNGNTYLE

SEQ ID NO: 19 - protein, 7 aa, synthetic construct
KVSNRFS

SEQ ID NO: 20 - protein, 9 aa, synthetic construct
FQGSHVPFT

SEQ ID NO: 21 - protein, 119 aa, synthetic construct
EVQLLESGGGLVQPGGSLRLSCAASGYTFTTYWITWVRQAPGKGLEWVSDIYPGSSISNYNEKFKSRFTISVDNSKNTLYLQMNSLRAEDTAVYYCAREDGYDAWFAYWGQGTLVTVSS

SEQ ID NO: 22 - protein, 112 aa, synthetic construct
DIQMTQSPSSLSASVGDRVTITCRSSQSILHSNGNTYLEWYQQKPGKAPKLLISKVSNRFSGVPSRFSGSGSGTDFTLTISSLQPEDFATYYCFQGSHVPFTFGQGTKLEIK

SEQ ID NO: 23 - protein, 330 aa, synthetic construct [sequence omitted]

SEQ ID NO: 24 - protein, 107 aa, synthetic construct [sequence omitted]

SEQ ID NO: 25 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

(19) United States
(12) Patent Application Publication
(10) Pub. No.: US 2025/0163135 A1
(43) Pub. Date: May 22, 2025

(54) BIOMARKERS FOR ALZHEIMER'S DISEASE TREATMENT

(71) Applicant: Eisai R&D Management Co., Ltd., Tokyo (JP)

(72) Inventors: Chad SWANSON, Nutley, NJ (US); Akihiko KOYAMA, Nutley, NJ (US); Michio KANEKIYO, Nutley, NJ (US); Michael IRIZARRY, Nutley, NJ (US); Lynn KRAMER, Nutley, NJ (US); June KAPLOW, Nutley, NJ (US); David VERBEL, Nutley, NJ (US); Shobha DHADDA, Nutley, NJ (US); Pallavi SACHDEV, Nutley, NJ (US); Larisa REYDERMAN, Nutley, NJ (US); Seiichi HAYATO,, Tokyo (JP); Ishani LANDRY, Nutley, NJ (US); Robert GORDON, Hatfield, Hertfordshire (GB)

(21) Appl. No.: 18/577,174

(22) PCT Filed: Jul. 8, 2022
(86) PCT No.: PCT/US2022/073576

§ 371 Date: Jan. 5, 2024

Related U.S. Application Data
(60) Provisional application No. 63/364,618, filed on May 12, 2022.
(60) Provisional application No. 63/269,372, filed on Mar. 15, 2022.
(60) Provisional application No. 63/306,028, filed on Feb. 2, 2022.
(60) Provisional application No. 63/264,551, filed on Nov. 24, 2021.
(60) Provisional application No. 63/263,928, filed on Nov. 11, 2021.
(60) Provisional application No. 63/263,255, filed on Oct. 29, 2021.
(60) Provisional application No. 63/203,444, filed on Jul. 22, 2021.
(60) Provisional application No. 63/220,434, filed on Jul. 9, 2021.

(51) Int. Cl. C07K 16/18; A61K 39/00; G01N 33/68

(52) U.S. Cl. CPC C07K 16/18; G01N 33/6896; A61K 2039/505; C07K 2317/24; G01N 2333/4709; G01N 2800/52

(57) ABSTRACT

Disclosed herein are method of diagnosing, selecting, monitoring, and treating subjects with Alzheimer's disease (AD) or suspected of having AD or another disorder associated with amyloid accumulation in the brain.

This application claims the benefit of and priority to U.S. Provisional Applications 63/220,434 filed Jul. 9, 2021; 63/203,444 filed Jul. 22, 2021; 63/263,255 filed Oct. 29, 2021; 63/263,928 filed Nov. 11, 2021; 63/264,551 filed Nov. 24, 2021; 63/306,028 filed Feb. 2, 2022; 63/269,372 filed Mar. 15, 2022; and 63/364,618 filed May 12, 2022, each entitled “BIOMARKERS FOR ALZHEIMER'S DISEASE TREATMENT,” the contents of which are expressly incorporated herein by reference in their entirety.

This invention was partially made with government support under Grant Nos. R01AG054029, R01AG061848, and 5U24AG057437-04, awarded by the National Instituted of Health. The government has certain rights in this invention.

Alzheimer's disease (AD) is a progressive, neurodegenerative disorder of unknown etiology and the most common form of dementia among older people. In 2006, there were 26.6 million cases of AD in the world (range: 11.4-59.4 million) (Brookmeyer, R., et al., Forecasting the global burden of Alzheimer's Disease. Alzheimer Dement. 2007; 3:186-91), while there were more than 5 million people in the United States reportedly living with AD (Alzheimer's Association, Alzheimer's Association report, 2010 Alzheimer's disease facts and figures. Alzheimer Dement. 2010; 6:158-94). By the year 2050, the worldwide prevalence of AD is predicted to grow to 106.8 million (range: 47.2-221.2 million), while in the United States alone the prevalence is estimated to be 11 to 16 million. (Brookmeyer, supra, and 2010 Alzheimer's disease facts and figures, supra).

The disease generally involves a global decline of cognitive function that progresses slowly and leaves end-stage subjects bedridden. AD subjects typically survive for only 3 to 10 years after symptom onset, although extremes of 2 and 20 years are known. (Hebert, L. E., et al., Alzheimer disease in the U.S. population: prevalence estimates using the 2000 census. Arch Neurol. 2003; 60:1119-1122.) AD is the seventh leading cause of all deaths in the United States and the fifth leading cause of death in Americans older than the age of 65 years, despite the fact that mortality due to AD is greatly underestimated because death certificates rarely attribute the cause of death to AD. (Alzheimer's Association. Alzheimer's Association report. 2010 Alzheimer's disease facts and figures. Alzheimer Dement. 2010; 6:158-94.)

AD represents a significant economic burden across industrialized countries with a substantial impact on healthcare systems and the public purse as well as on subjects and their families. In the United States alone, total payments for 2010 were estimated at $172 billion, including $123 billion for Medicare and Medicaid.

Histologically, the disease is characterized by neuritic plaques, found primarily in the association cortex, limbic system and basal ganglia. The major constituent of these plaques is amyloid beta peptide (Aβ). Aβ exists in various conformational states—monomers, oligomers, protofibrils, and insoluble fibrils. Details of the mechanistic relationship between onset of Alzheimer's disease and Aβ production is unknown. However, some anti-Aβ antibodies are undergoing clinical study now as potential therapeutic agents for Alzheimer's disease.

Despite the recent development of treatments for AD, including those targeting Aβ, there remains a need for better monitoring of treatments, including non-invasive assays to evaluate treatment efficacy and to calibrate treatment regimens in subjects. Currently, disease monitoring is largely dependent upon assays that are expensive and can increase the risk of complications to the subject Aβ positron emission tomography (PET) and cerebrospinal fluid (CSF) biomarker assays.

Accordingly, disclosed herein are improved methods of selecting, monitoring and treating patients with AD. In some embodiments, a patient is selected for treatment by
a. measuring a concentration of amyloid β 1-42 (Aβ42) and a concentration of amyloid β 1-40 (Aβ40) in a blood sample obtained from the subject to determine an Aβ 42 to Aβ40 (Aβ42/40 ratio);
b. optionally, measuring a concentration of phosphorylated tau181 (p-tau181) in the blood sample obtained from the subject;
c. selecting a patient for treatment who has an Aβ42/40 ratio below a threshold (e.g., a threshold of about 0.092) and, optionally, also has a p-tau181 concentration above a threshold.

In various embodiments, the methods comprise treating Alzheimer's disease (AD) in a subject having or suspected of having AD, comprising
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-amyloid β (Aβ) protofibril antibody;
d. measuring the concentration of Aβ42 and Aβ40 in a second blood sample obtained from the subject after the first sampling to determine a second Aβ42/40 ratio;
e. optionally, measuring a second level of p-tau181 in the second blood sample obtained from the subject;
and
f. administering a second therapeutically effective dose comprising the same or a lower amount of the anti-Aβ protofibril antibody than in the first dose to the subject having i) an elevated second ratio relative to the first ratio and/or ii) a second level of p-tau181 that is lower than the first level of p-tau181.

In some embodiments, more than one first dose and more than one second dose of the anti-Aβ protofibril antibody is administered, wherein the second doses are administered at a lower amount and/or a reduced frequency relative to the first doses.

In some embodiments, the methods comprise treating AD in a subject having or suspected of having AD, comprising
a. measuring a concentration of Aβ42 and a concentration of Aβ40 in a first blood sample obtained from the subject to determine a first ratio of Aβ42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-Aβ protofibril antibody;
d. measuring the concentration of Aβ42 and Aβ40 in a second blood sample obtained from the subject after the first sampling to determine a second Aβ42/40 ratio;
e. optionally, measuring a second level of p-tau181 in the second blood sample obtained from the subject; and
f. administering a second therapeutically effective dose comprising a higher amount of the anti-Aβ protofibril antibody than in the first dose or a different treatment for AD to the subject having i) the same or a reduced second ratio relative to the first ratio and/or ii) a second level of p-tau181 that is the same as or higher than the first level of p-tau181.

In some embodiments, the methods comprise reducing brain amyloid beta in a subject having or suspected of having AD, comprising
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-amyloid β (Aβ) protofibril antibody;
d. measuring the concentration of amyloid β 1-42 (Aβ42) and amyloid β 1-40 (Aβ40) in a second blood sample obtained from the subject after the first sampling to determine a second Aβ42/40 ratio;
e. optionally, measuring a second level of p-tau181 in the second blood sample obtained from the subject;
and
f. administering a second therapeutically effective dose comprising the same or a lower amount of the anti-Aβ protofibril antibody than in the first dose to the subject having i) an elevated second ratio relative to the first ratio and/or ii) a second level of p-tau181 that is lower than the first level of p-tau181,
thereby reducing brain amyloid beta in the subject.

In some embodiments, the methods comprise reducing brain amyloid beta in a subject having or suspected of having AD, comprising
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-amyloid β (Aβ) protofibril antibody;
d. measuring the concentration of amyloid β 1-42 (Aβ42) and amyloid β 1-40 (Aβ40) in a second blood sample obtained from the subject after the first sampling to determine a second Aβ42/40 ratio;
e. optionally, measuring a second level of p-tau181 in the second blood sample obtained from the subject;
and
f. administering a second therapeutically effective dose comprising a higher amount of the anti-Aβ protofibril antibody than in the first dose to the subject having i) the same or a reduced second ratio relative to the first ratio and/or ii) a second level of p-tau181 that is the same as or higher than the first level of p-tau181,
thereby reducing brain amyloid beta in the subject.

In some embodiments, the methods comprise monitoring treatment efficacy in a subject having or suspected of having AD, comprising:
a. administering to the subject a therapeutically effective dose of an anti-Aβ protofibril antibody;
b. measuring the concentration of Aβ42 and Aβ40 in a blood sample obtained from the subject to determine a Aβ42/40 ratio;
c. optionally, measuring the level of phosphorylated tau181 (p-tau181) in the blood sample;
d. comparing the Aβ42/40 ratio of the sample to a ratio in a sample from the patient prior to treatment or in a control, wherein the same or a higher Aβ42/40 ratio after treatment indicates an effective treatment; and
optionally, comparing the level of p-tau181 in a sample from the patient prior to treatment or in a control, wherein a reduced level of p-tau181 after treatment indicates an effective treatment.

In some embodiments, the methods comprise treating pre-Alzheimer's disease (pre-AD) in a subject, comprising:
a. measuring a concentration of amyloid β 1-42 (Aβ42) and a concentration of amyloid β 1-40 (Aβ40) in a blood sample obtained from the subject to determine a ratio of Aβ42 to Aβ40 (Aβ42/40 ratio); and
b. administering a treatment comprising a therapeutically effective dose of an anti-amyloid β (Aβ) protofibril antibody to the subject having an Aβ42/40 ratio below a threshold of about 0.092,
wherein the anti-Aβ protofibril antibody comprises a heavy chain variable region comprising an amino acid sequence of SEQ ID NO: 7 and a light chain variable region comprising an amino acid sequence of SEQ ID NO: 8, and wherein the subject exhibits at least one biomarker of AD (e.g., an Aβ42/40 ratio below a threshold of about 0.092) but is cognitively normal.

### BRIEF DESCRIPTION OF THE DRAWINGS

FIG. 1: Summary table of patient demographics and dosing regimen.

FIG. 2: Mean change in Aβ42/40 ratio by core treatment group and visit during Core, Gap and Open Label Extension (OLE) phases.

FIG. 3: Mean change in Aβ42/40 ratio and PET SUVR by core treatment group during Core, Gap and OLE phases.

FIG. 4: Summary table of change in PET SUVr and Plasma Aβ42/40 ratio during Core and OLE phases.

FIG. 5: Mean change in Aβ42/40 ratio and PET SUVR by core treatment group during OLE.

FIG. 6: Mean change from Core baseline measured by PET SUVr is negatively correlated with mean change from Core baseline in Aβ42/40 ratio.

FIG. 7: Mean change from OLE baseline measured by PET SUVr is negatively correlated with mean change from OLE baseline in Aβ42/40 ratio.

FIG. 8: Individual plasma Aβ42/40 ratio and PET SUVr changes plotted for all doses during Core phase.

FIG. 9: Individual plasma Aβ42/40 ratio and PET SUVr changes plotted for all doses during OLE phase.

FIG. 10: Individual subject data indicating correlation of PET SUVr with ADCOMS during Core, Gap, & OLE phases.

FIG. 11: Individual subject data indicating correlation of Aβ42/40 ratio with ADCOMS during Core, Gap, & OLE phases.

FIG. 12: Modeling data of Aβ42/40 ratio vs dosage in subjects who continued or discontinued treatment beyond 18 months.

FIG. 13: Modeling data of amyloid PET SUVr vs dosage in subjects who continued or discontinued treatment beyond 18 months.

FIG. 14: Individual Aβ42/40 ratio and amyloid PET values (right) and summary plot (left) prior to treatment. Subjects in the AHEAD 3-45 were grouped in one of two sister trials: Aβ intermediate amyloid (approximately 20-40 centiloids) and A45 elevated amyloid (>40 centiloids).

FIG. 15: Population correlation plot of ADCOMS vs. Plasma Aβ42/40 ratio during core study.

FIG. 16: Individual correlation plot of ADCOMS vs. Plasma Aβ42/40 ratio during core study.

FIG. 17: Model used to describe the relationship between the lecanemab concentration and the Aβ42/40 ratio change time course.

FIG. 18: Model used to describe the relationship between CFB for plasma Aβ42/40 ratio and clinical efficacy endpoints.

FIG. 19: Model used to describe the relationship between serum lecanemab concentration and the amyloid SUVr reduction time course.

FIG. 20: Correlation plot using patient data from the core study of the change from baseline of plasma Aβ42/40 ratio and amyloid PET SUVr.

FIG. 21: Mean change from OLE baseline measured by PET SUVr and mean change from OLE baseline in Aβ42/40 ratio; analysis includes additional patient data.

FIG. 22: Population correlation plot of plasma Aβ42/40 ratio relative to baseline vs. CDR-SB during Core study.

FIG. 23: Population correlation plot of plasma Aβ42/40 ratio relative to baseline vs. ADAS-Cog during Core study.

FIG. 24: Mean change in Aβ42/40 ratio and PET SUVR by core treatment group during Core, Gap and OLE phases; analysis includes additional patient data.

FIG. 25: Individual subject data indicating correlation of PET SUVr with CDR-SB during Core, Gap, & OLE phases; analysis includes additional patient data.

FIG. 26: Individual subject data indicating correlation of PET SUVr with ADCOMS during Core, Gap, & OLE phases; analysis includes additional patient data.

FIG. 27: Individual subject data indicating correlation of PET SUVr with ADAS-Cog during Core, Gap, & OLE phases; analysis includes additional patient data.

FIG. 28: Individual subject data indicating correlation of Aβ42/40 ratio with CDR-SB during Core, Gap, & OLE phases; analysis includes additional patient data.

FIG. 29: Individual subject data indicating correlation of Aβ42/40 ratio with ADCOMS during Core, Gap, & OLE phases; analysis includes additional patient data.

FIG. 30: Individual subject data indicating correlation of Aβ42/40 ratio with ADAS-Cog during Core, Gap, & OLE phases; analysis includes additional patient data.

FIG. 31: Mean change from baseline in Aβ42/40 ratio by treatment group during Core phase.

FIG. 32: Mean change from OLE baseline in Aβ42/40 ratio by treatment group during OLE phase.

FIG. 33: Individual PET SUVr and plasma Aβ42/40 ratio change from baseline plotted for all doses during Core phase.

FIG. 34: Model-predicted SUVr and plasma Aβ42/40 ratio and p-tau181 following 18 months treatment with lecanemab at 10 mg/kg biweekly or 10 mg/kg monthly FIG. 35: Model-predicted SUVr and plasma Aβ42/40 ratio and p-tau181 following continuous 10 mg/kg biweekly with or without treatment discontinuation.

### DETAILED DESCRIPTION

The “amyloid hypothesis” proposes that amyloid β (Aβ) peptides play a central role in the pathogenesis of AD. Specifically, it is hypothesized that neurodegeneration in AD may be caused by deposition of Aβ plaques in brain tissue due to an imbalance between Aβ production and Aβ clearance, leading to formation of neurofibrillary tangles containing tau protein. Aβ peptides generally exist in a dynamic continuum of conformational states such that species tend to progress from monomeric Aβ, to soluble Aβ assemblies that include a range of low molecular weight oligomers to higher molecular weight protofibrils, and finally to insoluble fibrils (plaques). Targeting these soluble and insoluble Aβ tangles and plaques may provide therapeutic benefit.

A number of immunotherapies have been developed with the intent to reduce the amount of insoluble Aβ fibrils deposited in the brain. However, a simple correlation between the quantity and progressive accumulation of insoluble amyloid plaques and the clinical course of AD has not been determined. While therapeutic strategies continue to focus on removal of insoluble amyloid plaques, an additional approach to therapy may include reducing the toxic Aβ aggregates, such as protofibrils, that may contribute to the neuronal degeneration characteristic of AD. (See, e.g., Dodort, J.-C. and May, P., “Overview on rodent models of Alzheimer's disease.” Curr. Protocols Neurosci. 2005; 9.22-1-9.22-6; Englund, H. et al., “Sensitive ELISA detection of amyloid-β protofibrils in biological samples.” J. Neurochem. 2007; 103:334-45; and Gotz, J. et al., “Transgenic animal models of Alzheimer's disease and related disorders: histopathology, behavior and therapy.” Mol. Psychiat. 2004; 9:664-83.)

In various embodiments, anti-Aβ protofibril antibodies, such as BAN2401 and other anti-Aβ protofibril antibodies, may be used to treat AD, e.g., by slowing AD progression in subjects, e.g., those at early stages of the disease when amyloid had been deposited in the brain but where the downstream neurodegenerative cascade thought to be triggered by the amyloid deposition was still relatively early in its course (i.e., limited brain tissue loss has been produced and associated clinical deficits are at a minimum).

In various embodiments, methods are disclosed herein for treating, monitoring treatment, and altering Aβ levels in patients receiving anti-Aβ protofibril antibodies, such as BAN2401, comprising evaluating a level of amyloid β 1-40 (Aβ40) and amyloid β 1-42 (Aβ42) to determine a ratio of Aβ 42 to Aβ40 (Aβ42/40 ratio). In some embodiments, the methods comprise measuring an Aβ42/40 ratio in a sample (e.g., a plasma sample) from a subject having or suspected of having AD before treatment and/or again in another sample during treatment (although it is to be understood that additional doses may be administered in between the sampling time points). In some embodiments, an increase in the Aβ42/40 ratio indicates treatment efficacy, e.g., a reduction in brain Aβ. In some embodiments, a subsequent dose of treatment is given after the second sampling if an elevated Aβ42/40 ratio is detected. In some embodiments, treatment may be titrated on the basis of the change in the Aβ42/40 ratio, e.g., dosage or treatment frequency may be reduced if an increase in the Aβ42/40 ratio is detected, alone or in combination with additional therapies such as BACE inhibitors or anti-tau antibodies. In some embodiments, dosage or treatment frequency may be increased, or an alternate treatment may be selected, if the Aβ42/40 ratio does not increase after the second sampling. In some embodiments, additional patient demographics, such as age and if the subject is a carrier of the apolipoprotein E ε4 gene allele, may be used to predict amyloid positivity (e.g. West et al, Mol Neurodegen (2021) 16-30, Jansen et al, JAMA (2015) 1924-1938, Ossenkoppele et al, JAMA (2015) 1939-1950). In some embodiments, an age and/or apolipoprotein E ε4 gene allele normalized measurement of the Aβ42/40 ratio from a subject is used to evaluate whether a sample (e.g., a plasma sample) from a subject indicates that the subject is amyloid positive or negative. For example, in some embodiments, a patient who is a carrier of an apolipoprotein E ε4 gene allele may be considered amyloid positive at a higher Aβ42/40 ratio than the ratio needed to indicate amyloid positivity in a subject who is not a carrier. Likewise, in another example, an older subject may be considered amyloid positive at a higher Aβ42/40 ratio than the ratio required to indicate positivity in a younger subject. In some embodiments, the Aβ42/40 is used in a Receiver Operating Characteristic (ROC) analysis to predict amyloid positivity. In some embodiments, additional patient demographics, such as age and if the subject is a carrier of an apolipoprotein E ε4 gene allele, may be used with the Aβ42/40 ratio in an ROC analysis to predict amyloid positivity. In some embodiments, the prediction of amyloid positivity in a patient is used to determine the dosage or frequency of treatment.

In some embodiments, the methods comprise measuring an Aβ42/40 ratio in a sample, e.g., a blood sample, from a subject having or suspected of having AD before treatment to identify a patient suitable for treatment and/or again in another sample during treatment to monitor treatment efficacy (although it is to be understood that additional doses may be administered in between the sampling time points). In some embodiments, treatment may be stopped and/or reduced (e.g., reduced frequency and/or dosage) if an increase in the Aβ42/40 ratio is detected between the first and second samplings. In some embodiments, after treatment has been stopped or reduced, a further measurement of the Aβ42/40 ratio may be made in a sample from the subject. In some embodiments, treatment is restarted, dosage is increased, and/or the frequency of administration is increased if a reduction in the Aβ42/40 ratio is detected. In some embodiments, the dosage or frequency of treatment is increased to return to the dosage and/or frequency used in a prior treatment, e.g., before a dose reduction and/or lengthening of the dose frequency had commenced. In some embodiments, the methods comprise measuring an Aβ42/40 ratio in a sample from a subject during treatment and again after stopping treatment or after the dosage or frequency of treatment has been reduced (it is to be understood that additional doses may be administered in between the sampling time points). In some embodiments, if a reduction in the Aβ42/40 ratio is detected, treatment is resumed, or the dosage or frequency of treatment is increased, in comparison to the dose or frequency during the period in which the ratio decreased. In some embodiments, multiple measurements may be made during a treatment prior to a decision to stop treatment and/or reduce treatment based on an elevated Aβ42/40 ratio (e.g., based on a trend showing increase in the Aβ42/40 ratio at each subsequent measurement). In some embodiments, multiple measurements may be taken after treatment has stopped or been reduced, and a decision to resume treatment and/or increase treatment may be taken based on a reduction in Aβ42/40 ratio (e.g., based on a trend showing a reduction in the Aβ42/40 ratio at each subsequent measurement). In some embodiments, following the resumption of treatment or the increased treatment regimen, one or more additional measurements may be made of the Aβ42/40 ratio in a sample from a subject. In some embodiments, treatment is continued if an increase in the Aβ42/40 ratio is observed in the subsequent measurements. In some embodiments, the measurement of the Aβ42/40 is done in conjunction with measuring one or more additional biomarkers (e.g., using a reduction in PET SUVr as an indicator of amyloid plaque reduction during and/or after treatment). In some embodiments, treatment may be stopped if a decrease in the Aβ42/40 ratio is detected between the first and a subsequent, e.g., second, third, or fourth, sampling. In some embodiments, treatment may be stopped due to a low therapeutic effect.

In some embodiments, any of the methods that comprise measuring an Aβ42/40 ratio may further comprise measuring one or more additional biomarkers, e.g., measuring phosphorylated tau (P-tau)(e.g., P-tau181). In some embodiments, the measurement of P-tau (e.g., P-tau181) is done in a sample, e.g., a blood sample, from a subject having or suspected of having AD before treatment and again in another sample during treatment (although it is to be understood that additional doses may be administered in between the sampling time points). In some embodiments, treatment may be stopped and/or reduced (e.g., reduced frequency and/or dosage) if a decrease in P-tau181 is detected between the first and second samplings. In some embodiments, after treatment has been stopped or reduced, a further measurement of the P-tau181 may be made in a sample from the subject. In some embodiments, treatment is restarted, dosage is increased, and/or the frequency of administration is increased if an increase in the P-tau181 is detected. In some embodiments, the dosage or frequency of treatment is increased to return to the dosage and/or frequency used in a prior treatment, e.g., before a dose reduction and/or lengthening of the dose frequency had commenced. In some embodiments, the methods comprise measuring P-tau181 in a sample from a subject during treatment and again after stopping treatment or after the dosage or frequency of treatment has been reduced (it is to be understood that additional doses may be administered in between the sampling time points). In some embodiments, if an increase in P-tau181 is detected, treatment is resumed, or the dosage or frequency of treatment is increased, in comparison to the dose or frequency during the period in which the level of P-tau181 decreased. In some embodiments, multiple measurements may be made during a treatment prior to stopping treatment and/or reducing treatment based on a decrease in P-tau181 (e.g., based on a trend showing a decrease in P-tau181 at each subsequent measurement). In some embodiments, multiple measurements may be taken after treatment has stopped or been reduced, before resuming treatment and/or increasing treatment based on an increase in P-tau181 (e.g., based on a trend showing an increase in P-tau181 at each subsequent measurement). In some embodiments, following the resumption of treatment or the increased treatment regimen, one or more additional measurements may be made of P-tau181 in a sample from a subject. In some embodiments, treatment is continued if a decrease in P-tau181 is observed in the subsequent measurements. In some embodiments, the measurement of P-tau181 is done in conjunction with measuring one or more additional biomarkers (e.g., using an increase in the Aβ42/40 ratio an indicator of amyloid plaque reduction during and/or after treatment).

In some embodiments, treatment is stopped and/or reduced (e.g., reduced frequency and/or dosage) if a decrease in P-tau (e.g., P-tau181) is detected between a first and second samplings in a subject and an increase in an Aβ42/40 ratio is detected in the samples. In some embodiments, treatment is resumed and/or increased (e.g., increased frequency and/or dosage) if an increase in P-tau (e.g., P-tau181) is detected after stopping and/or reducing an initial treatment in a subject and a decrease in an Aβ42/40 ratio is detected.

In some embodiments, treatment may be stopped if an increase in P-tau181 is detected between the first and a subsequent, e.g., second, third, or fourth, sampling. In some embodiments, treatment may be stopped due to a low therapeutic effect.

In some embodiments, provided herein is a method of reducing and/or slowing clinical decline in a subject, e.g., one having Pre-AD or early Alzheimer's disease, comprising administering a therapeutically effective amount of at least one anti-Aβ protofibril antibody (e.g., BAN2401) to a patient having an Aβ42/40 ratio less than about 0.092. In some embodiments, the anti-Aβ protofibril antibody (e.g., BAN2401) is administered in a therapeutically effective amount to increase the Aβ42/40 ratio above about 0.092. In some embodiments, increasing the Aβ42/40 ratio slows the cognitive decline of a patient (e.g., one having pre-AD or early AD) relative to the decline in the absence of treatment.

In some embodiments, a treatment comprises administering intravenously an anti-Aβ protofibril antibody before switching to a maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative, before switching to a maintenance dose. In some embodiments, a subject is switched to a maintenance dose without an initial titrating step to the maintenance dose. In some embodiments, a subject is switched to a maintenance dose with at least one titrating step to the maintenance dose, e.g., the subject's dosage or frequency of administration may be reduced in multiple steps until achieving a final maintenance dosing regime (e.g., a stepwise reduction from a subcutaneous treatment dosing regimen of 720 mg weekly to a maintenance dosing regimen of 360 mg weekly or 720 mg biweekly via intermediate dosing at intermediate amounts or time periods such as 540 mg weekly or 720 mg every 10 days). In some embodiments, a subject's maintenance dose is administered at the same amount and/or frequency as the dose during the treatment period. In some embodiments, a subject's maintenance dose is 50% of the dose during the treatment period. An anti-Aβ protofibril antibody, such as BAN2401, may be formulated in a pharmaceutical composition as disclosed in PCT/IB2021/000155 (WO2021/186245), which is incorporated herein by reference. In some embodiments, the composition comprises 80 mg/mL to 120 mg/mL BAN2401, 240 mM to 360 mM arginine, 0.03% w/v to 0.08% w/v polysorbate 80, and 30 mM to 70 mM citrate buffer. In some embodiments, the arginine is arginine, arginine hydrochloride, or a combination thereof. In some embodiments, the composition comprises a liquid dosage form comprising 100 mg/mL BAN2401, 50 mmol/L citrate, 350 mmol/L arginine, and 0.05% polysorbate 80. In some embodiments, the composition comprises 80 mg/mL to 240 mg/mL BAN2401, 140 mM to 260 mM arginine hydrochloride, 0.01% w/v to 0.1% w/v polysorbate 80, and 15 mM to 35 mM histidine buffer. In some embodiments, the composition comprises a liquid dosage form comprising 100 mg/mL BAN2401, 25 mmol/L histidine, 200 mmol/L arginine, and 0.05% polysorbate 80. In some embodiments, treatment is continued until a desired improvement in one or more biomarker or other treatment outcome measure is achieved, e.g., when an increase in the Aβ42/40 ratio is observed in a sample (e.g., a plasma sample) relative to the ratio in a sample taken from the subject before treatment, e.g., before 18 months of treatment. In some embodiments, a maintenance dosing regimen may further comprise one or more additional treatments in addition to an anti-Aβ protofibril antibody, e.g., it may comprise administering E2814.

In some embodiments, a treatment comprises subcutaneously administering an anti-Aβ protofibril antibody, e.g., BAN2401, before switching to a subcutaneous maintenance dose. In some embodiments, a treatment comprises subcutaneously administering BAN2401 weekly, e.g., weekly subcutaneous injection of 720 mg in two concurrent, e.g., sequential, injections of 360 mg (2×1.8 mL of 400 mg/2 mL) of the subcutaneous formulation, e.g., until a patient is amyloid-negative or e.g., for at least 18 months. In some embodiments, a treatment comprises subcutaneously administering BAN2401 weekly, e.g., at a dose of 720 mg, e.g., for at least 18 months or e.g., until a patient is amyloid-negative, and then switching to a maintenance dose. In some embodiments, a treatment comprises subcutaneously administering BAN2401 weekly, e.g., at a dose of 720 mg, e.g., for at least 18 months or e.g., until a patient is amyloid-negative, before switching to a weekly, subcutaneous maintenance dose, e.g., a dose of 360 mg. In some embodiments, a treatment comprises subcutaneously administering BAN2401 weekly, e.g., at a dose of 720 mg, e.g., for at least 18 months or e.g., until a patient is amyloid-negative, before switching to a biweekly subcutaneous maintenance dose, e.g., a dose of 720 mg. In some embodiments, a treatment comprises subcutaneously administering BAN2401 weekly, e.g., at a dose of 720 mg, e.g., for at least 18 months or e.g., until a patient is amyloid-negative, before switching to a monthly subcutaneous maintenance dose, e.g., a dose of 720 mg. In some embodiments, a subject's maintenance dose is administered at the same amount and/or frequency as the dose during the treatment period. In some embodiments, a subject's maintenance dose is 50% of the dose during the treatment period. In some embodiments, BAN2401, is formulated as disclosed in PCT/IB2021/000155 (WO2021/186245), which is incorporated herein by reference. In some embodiments, the composition comprises 80 mg/mL to 240 mg/mL BAN2401, 140 mM to 260 mM arginine hydrochloride, 0.01% w/v to 0.1% w/v polysorbate 80, and 15 mM to 35 mM histidine buffer. In some embodiments, the composition comprises a liquid dosage form comprising 200 mg/mL BAN2401, 25 mmol/L histidine, 200 mmol/L arginine, and 0.05% polysorbate 80. In some embodiments, a treatment comprises subcutaneously administering BAN2401 twice weekly, e.g., at 720 mg per dose, e.g., for at least 18 months or e.g., until a patient is amyloid-negative. In some embodiments, treatment is continued until a desired improvement in one or more biomarker or other treatment outcome measure is achieved, e.g., when an increase in the Aβ42/40 ratio is observed in a sample (e.g., a plasma sample) relative to the ratio in a sample taken from the subject before treatment, e.g., before 18 months of treatment.

In some embodiments, following a treatment period, a maintenance dose is administered. In some embodiments, a treatment comprises administering intravenously an anti-Aβ protofibril antibody before switching to an intravenous maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative, before switching to an intravenous maintenance dose. In some embodiments, a treatment comprises administering intravenously an anti-Aβ protofibril antibody before switching to a subcutaneous maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative, before switching to a subcutaneous maintenance dose, e.g., 720 mg administered weekly or biweekly, or 360 mg administered weekly.

In some embodiments, the maintenance dose is administered intravenously, e.g., after an intravenous treatment period as disclosed above. In some embodiments, an intravenous maintenance dose, e.g., a dosing of 10 mg/kg BAN2401, is administered every week, two weeks, every month, every two months, or every three months (quarterly). In some embodiments, the intravenous maintenance dose is administered every two weeks. In some embodiments, the intravenous maintenance dose is administered every four weeks. In some embodiments, the intravenous maintenance dose is administered every six weeks. In some embodiments, the intravenous maintenance dose is administered every eight weeks (2 months). In some embodiments, the intravenous maintenance dose is administered every three months (quarterly). In some embodiments, the intravenous maintenance dose is administered every 24 weeks (every six months or semi-annually). In some embodiments, the intravenous maintenance dose is 2.5 mg/kg-10 mg/kg. In some embodiments, the maintenance dose is administered as a biweekly, intravenous dose of 10 mg/kg BAN2401. In some embodiments, the maintenance dose is administered as an intravenous dose of 10 mg/kg every four weeks (monthly). In some embodiments, the maintenance dose is administered as an intravenous dose of 10 mg/kg every six weeks. In some embodiments, the maintenance dose is administered as an intravenous dose of 10 mg/kg every eight weeks (2 months). In some embodiments, the maintenance dose is administered as an intravenous dose of 10 mg/kg every twelve weeks (every three months or quarterly). In some embodiments, the maintenance dose is administered as an intravenous dose of 10 mg/kg every 24 weeks (every six months or semi-annually). In some embodiments, a treatment comprises administering intravenously an anti-Aβ protofibril antibody at 10 mg/kg, biweekly, e.g., for at least 18 months or e.g., until a patient is amyloid-negative, before switching to a weekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a biweekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a monthly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to an intravenous maintenance dose every six weeks. In some embodiments, a treatment comprises administering intravenously an anti-Aβ protofibril antibody at 10 mg/kg, biweekly, e.g., for at least 18 months or e.g., until a patient is amyloid-negative, before switching to an intravenous maintenance dose every eight weeks. In some embodiments, a treatment comprises administering intravenously an anti-Aβ protofibril antibody at 10 mg/kg, biweekly, e.g., for at least 18 months or e.g., until a patient is amyloid-negative, before switching to a quarterly intravenous maintenance dose.

In some embodiments, a maintenance dose is administered subcutaneously (e.g., as one or more subcutaneous injections). In some embodiments, a treatment comprises administering intravenously an anti-Aβ protofibril antibody before switching to a subcutaneous maintenance dose. In other embodiments, a treatment comprises subcutaneously administering an anti-Aβ protofibril antibody before switching to an intravenous maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative, before switching to a subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a weekly subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a weekly, 360 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a weekly, 720 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a biweekly, 720 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a monthly, 720 mg, subcutaneous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a quarterly, 720 mg, subcutaneous maintenance dose.

In some embodiments, a patient will begin treatment comprising administering intravenously an anti-Aβ protofibril antibody, e.g., at a dose of 10 mg/kg, then switch to a treatment (e.g., a maintenance treatment) comprising subcutaneously administering an anti-Aβ protofibril antibody, e.g., at a dose of 720 mg. In some embodiments, a patient will begin treatment comprising administering intravenously an anti-Aβ protofibril antibody at 10 mg/kg, biweekly, then switch to a treatment comprising subcutaneously administering BAN2401 weekly, e.g., at a dose of 720 mg, e.g., for a total treatment period of at least 18 months or e.g., until the patient is amyloid-negative. In some embodiments, a patient will begin treatment comprising administering intravenously an anti-Aβ protofibril antibody at 10 mg/kg, biweekly, then switch to a treatment comprising subcutaneously administering BAN2401 weekly, e.g., at a dose of 720 mg, before switching to a weekly, 360 mg, subcutaneous maintenance dose. In some embodiments, a patient will begin treatment comprising administering intravenously an anti-Aβ protofibril antibody at 10 mg/kg, biweekly, then switch to a treatment comprising subcutaneously administering BAN2401 weekly, e.g., at a dose of 720 mg, before switching to a monthly, 720 mg, subcutaneous maintenance dose.

In some embodiments, the maintenance dose is administered as a subcutaneous injection of the anti-Aβ protofibril antibody (e.g., BAN2401). In some embodiments, the maintenance dose is administered as a weekly subcutaneous injection of the subcutaneous formulation of the anti-Aβ protofibril antibody. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a quarterly, subcutaneous injection of 720 mg comprising two concurrent, e.g., sequential, injections of 360 mg (2×1.8 mL of 400 mg/2 mL) of the subcutaneous formulation. In some embodiments, the subcutaneous maintenance dose is administered weekly. In some embodiments, the subcutaneous maintenance dose is administered every two weeks. In some embodiments, the subcutaneous maintenance dose is administered every four weeks (monthly). In some embodiments, the subcutaneous maintenance dose is administered every six weeks. In some embodiments, the subcutaneous maintenance dose is administered every eight weeks (2 months). In some embodiments, the subcutaneous maintenance dose is administered every three months (twelve weeks or quarterly). In some subcutaneous embodiments, the maintenance dose is administered weekly, every two weeks, every 4 weeks, every 6 weeks, every 8 weeks, every 10 weeks, every 12 weeks, every 16 weeks, every 24 weeks, every 48 weeks, monthly, every 2 months, every 3 months, every 4 months, every 6 months, or every 12 months. In some embodiments, the subcutaneous maintenance dose comprises an anti-Aβ protofibril antibody at a dose of 300 mg to 800 mg, 300 mg to 400 mg, 400 mg to 500 mg, 400 mg to 450 mg, 450 mg to 500 mg, 500 mg to 600 mg, 500 mg to 550 mg, 550 mg to 600 mg, 600 mg to 700 mg, 600 mg to 650 mg, 650 mg to 700 mg, 700 mg to 800 mg, 700 mg to 750 mg, or 750 mg to 800 mg. In some embodiments, the maintenance dose is 300 mg, 310 mg, 320 mg, 330 mg, 340 mg, 350 mg, 360 mg, 370 mg, 380 mg, or 390 mg. In some embodiments, the maintenance dose is 400 mg, 410 mg, 420 mg, 430 mg, 440 mg, 450 mg, 460 mg, 470 mg, 480 mg, or 490 mg. In some embodiments, the maintenance dose is 500 mg, 510 mg, 520 mg, 530 mg, 540 mg, 550 mg, 560 mg, 570 mg, 580 mg, or 590 mg. In some embodiments, the maintenance dose is 600 mg, 610 mg, 620 mg, 630 mg, 640 mg, 650 mg, 660 mg, 670 mg, 680 mg, or 690 mg. In some embodiments, the maintenance dose is 700 mg, 710 mg, 720 mg, 730 mg, 740 mg, 750 mg, 760 mg, 770 mg, 780 mg, or 790 mg. In some embodiments, the maintenance dose is 800 mg to 1600 mg, 800 mg to 1000 mg, 800 mg to 900 mg, 900 mg to 1000 mg, 1000 mg to 1200 mg, 1000 mg to 1100 mg, 1100 mg to 1200 mg, 1200 mg to 1400 mg, 1200 mg to 1300 mg, 1300 mg to 1400 mg, 1400 mg to 1600 mg, 1400 mg to 1500 mg, or 1500 mg to 16000 mg. In some embodiments, the maintenance dose is 800 mg, 820 mg, 840 mg, 860 mg, 880 mg, 900 mg, 920 mg, 940 mg, 960 mg, or 980 mg. In some embodiments, the maintenance dose is 1000 mg, 1020 mg, 1040 mg, 1060 mg, 1080 mg, 1100 mg, 1120 mg, 1140 mg, 1160 mg, or 1180 mg. In some embodiments, the maintenance dose is 1200 mg, 1220 mg, 1240 mg, 1260 mg, 1280 mg, 1300 mg, 1320 mg, 1340 mg, 1360 mg, or 1380 mg. In some embodiments, the maintenance dose is 1400 mg, 1420 mg, 1440 mg, 1460 mg, 1480 mg, 1500 mg, 1520 mg, 1540 mg, 1560 mg, or 1580 mg. In some embodiments, the maintenance dose is provided in a single administration, e.g., administered as a single subcutaneous injection of 720 or 1440 mg, or in two or more administrations, e.g., two concurrent administrations of 360 mg for a total of 720 mg or two administrations of 720 mg for a total of 1440 mg, or four administrations of 360 mg for a total of 1440 mg. In some embodiments, the maintenance dose is 440 mg. In some embodiments, the maintenance dose is 580 mg. In some embodiments, the maintenance dose is administered as a single administration of 720 mg or two administrations of 360 mg. In some embodiments, the maintenance dose is 1440 mg. In some embodiments, the maintenance dose is administered as a weekly, subcutaneous injection of 720 mg. In some embodiments, the maintenance dose is administered as a weekly, subcutaneous injection of 360 mg. In some embodiments, the maintenance dose is administered as a biweekly, subcutaneous injection of 720 mg. In some embodiments, the maintenance dose is administered as a biweekly, subcutaneous injection of 1440 mg. In some embodiments, the maintenance dose is provided in a single, biweekly administration of 1440 mg comprising two concurrent, e.g., sequential administrations of 720 mg of the subcutaneous formulation for a total of 1440 mg.

In some embodiments, a treatment comprises subcutaneously administering an anti-Aβ protofibril antibody, e.g., BAN2401, before switching to an intravenous maintenance dose. In some embodiments, a treatment comprises subcutaneously administering BAN2401 weekly, e.g., a subcutaneous injection of 720 mg comprising two concurrent, e.g., sequential, injections of 360 mg (2×1.8 mL of 400 mg/2 mL), e.g., until a patient is amyloid-negative or e.g., for at least 18 months. In some embodiments, a treatment comprises subcutaneously administering BAN2401 weekly, e.g., at a dose of 720 mg, e.g., for at least 18 months or e.g., until a patient is amyloid-negative, and then switching to a maintenance dose. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg weekly. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg biweekly. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg monthly. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg every six weeks. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg every eight weeks. In some embodiments, a treatment comprises subcutaneously administering BAN2401 weekly, e.g., at a dose of 720 mg, e.g., for at least 18 months or e.g., until a patient is amyloid-negative, before switching to an intravenous maintenance dose of 10 mg/kg quarterly. In some embodiments, a subject's maintenance dose is administered at the same amount and/or frequency as the dose during the treatment period. In some embodiments, a subject's maintenance dose is 50% of the dose during the treatment period.

In some embodiments, the maintenance dose is administered intravenously, e.g., after an intravenous treatment period as disclosed above. In some embodiments, an intravenous maintenance dose, e.g., a dosing of 10 mg/kg BAN2401, is administered every week, two weeks, every month, every two months, or every three months (quarterly). In some embodiments, the intravenous maintenance dose is administered every two weeks. In some embodiments, the intravenous maintenance dose is administered every four weeks. In some embodiments, the intravenous maintenance dose is administered every six weeks. In some embodiments, the intravenous maintenance dose is administered every eight weeks (2 months). In some embodiments, the intravenous maintenance dose is administered every three months (quarterly). In some embodiments, the intravenous maintenance dose is administered every 24 weeks (every six months or semi-annually). In some embodiments, the intravenous maintenance dose is 2.5 mg/kg-10 mg/kg. In some embodiments, the maintenance dose is administered as a biweekly, intravenous dose of 10 mg/kg BAN2401. In some embodiments, the maintenance dose is administered as an intravenous dose of 10 mg/kg every four weeks (monthly). In some embodiments, the maintenance dose is administered as an intravenous dose of 10 mg/kg every six weeks. In some embodiments, the maintenance dose is administered as an intravenous dose of 10 mg/kg every eight weeks (2 months). In some embodiments, the maintenance dose is administered as an intravenous dose of 10 mg/kg every twelve weeks (every three months or quarterly). In some embodiments, the maintenance dose is administered as an intravenous dose of 10 mg/kg every 24 weeks (every six months or semi-annually). In some embodiments, a treatment comprises administering intravenously an anti-Aβ protofibril antibody at 10 mg/kg, biweekly, e.g., for at least 18 months or e.g., until a patient is amyloid-negative, before switching to a weekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a biweekly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to a monthly intravenous maintenance dose. In some embodiments, a treatment comprises administering intravenously an anti-Aβ protofibril antibody at 10 mg/kg, biweekly, e.g., for at least 18 months or e.g., until a patient is amyloid-negative, before switching to an intravenous maintenance dose every six weeks. In some embodiments, a treatment comprises administering intravenously an anti-Aβ protofibril antibody at 10 mg/kg, biweekly, e.g., for at least 18 months or e.g., until a patient is amyloid-negative, before switching to an intravenous maintenance dose every eight weeks. In some embodiments, a treatment comprises administering intravenously an anti-Aβ protofibril antibody at 10 mg/kg, biweekly, e.g., for at least 18 months or e.g., until a patient is amyloid-negative, before switching to a quarterly intravenous maintenance dose.

In some embodiments, a patient starts on an intravenous maintenance dose, e.g., a dosing of 10 mg/kg BAN2401 as disclosed above before switching to a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation. In some embodiments, a patient starts on a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation before switching to an intravenous maintenance dose, e.g., a dosing of 10 mg/kg BAN2401 as disclosed above.

In some embodiments, a patient is moved back from a maintenance dose to the initial treatment dose if the patient is determined to no longer be amyloid negative, e.g., as assessed by measuring an Aβ42/40 ratio below about 0.092 in a blood sample taken after switching to a maintenance dose and/or as determined by PET SUVr. In some embodiments, a patient's treatment is discontinued if the patient is determined to no longer be amyloid negative, e.g., as assessed by measuring an Aβ42/40 ratio below 0.092 in a blood sample taken after switching to a maintenance dose.

In some embodiments, the maintenance dose is administered at least every three months (e.g., quarterly) or every twelve weeks. In some embodiments, after switching to a maintenance dose, the Aβ42/40 ratio is measured in a sample (e.g., a plasma sample) from the subject. In some embodiments, the maintenance dose and/or frequency is selected to maintain an Aβ42/40 ratio achieved after the completion of the initial treatment (e.g., after 18 months of treatment). In some embodiments, the maintenance dose and/or frequency is selected to maintain a Aβ42/40 ratio at or above 0.092-0.094 (e.g., at or above 0.092). In some embodiments, the maintenance dose and/or frequency is selected to maintain a Aβ42/40 ratio above 0.092. In some embodiments, the maintenance dose is continued if the Aβ42/40 ratio remains unchanged or increases. In some embodiments, a patient's amyloid level may be monitored during the treatment with the maintenance dose, e.g., by a blood biomarker. In some embodiments, a patient's amyloid level may be monitored during the treatment with the maintenance dose by one or more biomarkers such as, but not limited to: (a) amyloid detected by PET scan from either a visual read or semiquantitative thresholds (SUVr or centiloid); (b) cerebrospinal fluid (CSF) Aβ 1-42, and/or Aβ1-42/1-40 ratio; and/or (c) blood biomarkers (such as plasma Aβ1-42, total tau (T-tau), and/or phosphorylated tau (P-tau)(e.g., P-tau181)). In some embodiments, a patient's biomarkers may be monitored at least once after switching to the maintenance dose. In some embodiments, a patient's biomarkers are evaluated at least 1 week, 2 weeks, 3 weeks, 1 month, 2 months, 3 months, 6 months, 12 month, 18 months, or 24 months after switching to the maintenance dose. In some embodiments, a subject is returned to the original dosing (e.g., 10 mg/kg BAN2401 biweekly) if one or more biomarkers worsen, e.g., if the Aβ42/40 ratio reduces relative the ratio measured in a sample at the end of the earlier treatment period (e.g., at 18 months after the start of treatment). In some embodiments, a subject is administered a higher dose (e.g., a 50% increase in the maintenance dose) if one or more biomarkers worsen, e.g., if the Aβ42/40 ratio reduces relative the ratio measured in a sample at the end of the earlier treatment period (e.g., at 18 months after the start of treatment). In some embodiments, a subject is administered a treatment at a higher frequency (e.g., a change from biweekly to weekly administration) if one or more biomarkers worsen, e.g., if the Aβ42/40 ratio reduces relative the ratio measured in a sample at the end of the earlier treatment period (e.g., at 18 months after the start of treatment).

In some embodiments, a subject's maintenance dose is the same as the dose during the treatment period. In some embodiments, a maintenance dose is selected (e.g., in conjunction with the evaluation of a change in the Aβ42/40 ratio) based on whether the patient is an ApoE4 carrier, e.g., with a greater increase in the Aβ42/40 ratio required to move from the initial treatment to a maintenance dose for a carrier than for a non-carrier. In some embodiments, the maintenance dose comprises two or more dosings, in which a first dosing is selected from the maintenance dose as exemplified above and a second and/or subsequent dosing comprising a lower amount and/or frequency of dosing than the first or previous dosing, respectively. In some embodiments, the switching to the second or subsequent dosing is determined based on one or more biomarkers as exemplified above, where the levels of the biomarkers are different from (e.g., improved over) the levels used in switching from an initial dose to the first dosing in the maintenance dose.

In some embodiments, after switching to a maintenance dose, a subject's biomarker levels will indicate increasing levels of amyloid in the brain. In some embodiments, after switching to a maintenance dose, a subject's biomarker levels, e.g. the plasma Aβ42/40 ratio, will began to decrease, indicating increasing levels of amyloid in the brain. In some embodiments, a subject on a maintenance dose will have a decrease in the Aβ42/40 ratio. In some embodiments, a subject is put on a maintenance dose chosen such that the subject will have a decrease in the Aβ42/40 ratio but the Aβ42/40 ratio will remain below the threshold for amyloid positivity, e.g. for at least one year (e.g., at least 1, 2, 3, 4, 5, 6, 7, 8, 9, or 10 years).

In some embodiments, after switching to a maintenance dose, a subject's biomarker levels, e.g. p-tau181, will began to increase, indicating increasing levels of amyloid in the brain. In some embodiments, a subject on a maintenance dose will have an increase in plasma p-tau181. In some embodiments, a subject on a maintenance dose will have an increase in p-tau181 but the level p-tau181 will remain above the threshold for amyloid positivity, e.g., for at least one year (e.g., at least 1, 2, 3, 4, 5, 6, 7, 8, 9, or 10 years).

In some embodiments, a patient's treatment is discontinued if a patient no longer has early AD, e.g., as assessed by cognitive evaluation, PET SUVr, and/or plasma biomarkers such as an Aβ42/40 ratio (e.g., if an Aβ42/40 ratio drops below 0.092 and/or an SUVr negativity increases above 1.17 as measured using florbetapir).

In some embodiments, a treatment is discontinued if a favorable biomarker level is achieved. In some embodiments, a treatment is discontinued if a favorable biomarker level is achieved after the completion of the initial treatment. In some embodiments, a treatment is discontinued if a favorable biomarker level is achieved and/or maintained (e.g., for a set period of time such as six months or a year) during a maintenance dosing. In some embodiments, a treatment is discontinued if a high Aβ42/40 ratio (e.g., an Aβ42/40 ratio at or about 0.09, 0.091, 0.092, 0.093, 0.094, 0.095, 0.096, 0.097, 0.099, 0.1) is achieved, e.g., after the completion of the initial treatment or during a maintenance dosing regimen. In some embodiments, a treatment is discontinued if an Aβ42/40 ratio at or above 0.092 is achieved. In some embodiments, a treatment is discontinued if an Aβ42/40 ratio above 0.092 is achieved. In some embodiments, a treatment is discontinued if an SUVr amyloid negativity level is at or below 1.17 as measured using florbetapir after the completion of the initial treatment or during a maintenance dosing regimen.

In some embodiments, a maintenance dose is discontinued if a favorable biomarker level is achieved after the completion of a set period of time on the maintenance treatment (e.g., six months or a year). In some embodiments, a maintenance dose is discontinued if a high Aβ42/40 ratio (e.g., an Aβ42/40 ratio at or about 0.09, 0.091, 0.092, 0.093, 0.094, 0.095, 0.096, 0.097, 0.099, 0.1) is achieved. In some embodiments, a maintenance dose is discontinued if an Aβ42/40 ratio at or above 0.092 is achieved. In some embodiments, a maintenance dose is discontinued if an Aβ42/40 ratio above 0.092 is achieved. In some embodiments, a maintenance dose is discontinued if the SUVr amyloid negativity level is at or below 1.17 as measured using florbetapir.

In some embodiments, a maintenance dose is discontinued if a favorable biomarker level is not maintained over the course of a maintenance treatment (e.g., if an Aβ42/40 ratio drops below about 0.092 and/or an SUVr negativity increases above 1.17 as measured using florbetapir). In some embodiments, a maintenance dose is discontinued if a favorable biomarker level is not maintained over the course of a maintenance treatment (e.g., if an Aβ42/40 ratio drops below 0.092 and/or an SUVr negativity increases above 1.17 as measured using florbetapir).

In some embodiments, a patient's amyloid level may be monitored for regression after treatment discontinuation, e.g., by a blood biomarker. In some embodiments, a patient's amyloid level may be monitored for regression after treatment discontinuation by one or more biomarkers such as, but not limited to: (a) amyloid detected by PET scan from either a visual read or semiquantitative thresholds (SUVr or centiloid); (b) cerebrospinal fluid (CSF) Aβ1-42, and/or Aβ 1-42/1-40 ratio; and/or (c) blood biomarkers (such as plasma Aβ 1-42, tau, total tau (T-tau), and/or P-tau (e.g., P-tau181)). In some embodiments, a patient's biomarkers may be monitored at least once after the discontinuation of treatment. In some embodiments, a patient's biomarkers are monitored at least 1 week, 2 weeks, 3 weeks, 1 month, 2 months, 3 months, 6 months, 12 month, 18 months, or 24 months after treatment discontinuation. In some embodiments, treatment is reinitiated if a patient's biomarker level becomes less favorable, e.g., a reduction in an Aβ42/40 ratio, e.g., to less than about 0.092. In some embodiments, treatment is reinitiated if a patient's biomarker level becomes less favorable, e.g., a reduction in an Aβ42/40 ratio, e.g., to less than 0.092.

In some embodiments, the maintenance dose is administered at least every three months (e.g., every three months, every two months, monthly, biweekly, or weekly). In some embodiments, the maintenance dose and/or frequency is selected to maintain a PET SUVr level achieved after the completion of the initial treatment. In some embodiments, the maintenance dose is selected to maintain a PET SUVr level at or below amyloid negativity (e.g. for florbetapir, PET SUVr of 1.17).

In some embodiments, the subject has been diagnosed with early AD. In some embodiments, the subject has been diagnosed as having mild cognitive impairment due to Alzheimer's disease-intermediate likelihood and/or has been diagnosed as having mild Alzheimer's disease dementia.

In some embodiments, the method of treatment comprises measuring the concentration of amyloid β 1-42 (Aβ42) and a concentration of amyloid β 1-40 (Aβ40) in a first blood sample obtained from the subject to determine a first ratio of Aβ42 to Aβ40 (Aβ42/40 ratio). In some embodiments, the subject is then administered a therapeutically effective dose of an anti-amyloid R (Aβ) protofibril antibody. In some embodiments, a second blood sample is obtained after the first sample to determine a second Aβ42/40 ratio. In some embodiments, a second blood sample is obtained from a subject after treatment has stopped or been reduced. In some embodiments, a change in the Aβ42/40 ratio is used to determine a second therapeutically effective dose. In some embodiments, a subject having an elevated second ratio relative to the first ratio is administered a second therapeutically effective dose comprising the same or a lower amount of the anti-Aβ protofibril antibody than in the first dose to the subject. In some embodiments, a subject having a lower second ratio relative to the first ratio is administered a second therapeutically effective dose comprising a higher amount of the anti-Aβ protofibril antibody than in the first dose. In some embodiments, a subject having a lower second ratio relative to the first ratio is administered a different treatment for AD. A first therapeutically effective dose may be administered multiple times (e.g., biweekly or monthly for 6-18 months) before changing to a second therapeutically effective dose or dosing regimen after measuring a second Aβ42/40 ratio. In some embodiments, a first therapeutically effective dose may be administered for at least 18 months before switching to a maintenance dose. In some embodiments, a first therapeutically effective dose may be administered until a patient is amyloid negative before switching to a maintenance dose. In some embodiments, a first therapeutically effective dose may be administered until a patient is amyloid negative (e.g., as measured by amyloid or tau positron emission tomography (PET), cerebrospinal fluid level of Aβ1-42 and/or Aβ1-42/1-40 ratio, cerebrospinal fluid level of total tau, cerebrospinal fluid level of neurogranin, cerebrospinal fluid level of neurofilament light peptide (NfL), and blood biomarkers as measured in the serum or plasma (e.g. levels of Aβ1-42, the ratio of two forms of amyloid-β peptide (Aβ1-42/1-40 ratio), plasma levels of plasma total tau (T-tau), levels of phosphorylated tau (P-tau) isoforms (including tau phosphorylated at 181 (P-tau181), 217 (P-tau217), and 231 (P-tau231)), glial fibrillary acidic protein (GFAP), and/or neurofilament light (NfL)) before switching to a maintenance dose. In some embodiments, a first therapeutically effective dose may be administered until a patient is amyloid negative, e.g., as measured by an Aβ42/40 ratio at or above 0.092-0.094 (e.g., at or above 0.092) or a florbetapir amyloid PET SUVr negativity at or below 1.17, before switching to a maintenance dose. In some embodiments, a first therapeutically effective dose may be administered until a patient is amyloid negative, e.g., as measured by an Aβ42/40 ratio above 0.092 or a florbetapir amyloid PET SUVr negativity at or below 1.17, before switching to a maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a maintenance dose.

In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to an intravenous maintenance dose (e.g., at 10 mg/kg, e.g., biweekly, or every 4, 6, 8, 10, or 12 weeks). In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a biweekly intravenous maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a monthly intravenous maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to an intravenous maintenance dose every six weeks. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to an intravenous maintenance dose every eight weeks. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to an intravenous maintenance dose every two months. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a quarterly intravenous maintenance dose.

In some embodiments, a first therapeutically effective dose comprises subcutaneously administering an anti-Aβ protofibril antibody at 720 mg (e.g., administering BAN2401 at 720 mg) weekly, e.g., for at least 18 months or e.g., until a patient is amyloid-negative before switching to a subcutaneous maintenance dose (e.g., at 720 mg, e.g., weekly, biweekly, or every 4, 6, 8, 10, or 12 weeks). In some embodiments, the maintenance dose is 360 mg weekly.

In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a weekly subcutaneous maintenance dose (e.g., at a dose of 720 mg). In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a weekly subcutaneous maintenance dose (e.g., at a dose of 360 mg). In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a biweekly subcutaneous maintenance dose (e.g., at a dose of 720 mg or at a dose of 360 mg). In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every month. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every six weeks. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every eight weeks. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a subcutaneous maintenance dose (e.g., at a dose of 720 mg or at a dose of 360 mg) every two months. In some embodiments, a first therapeutically effective dose comprises administering intravenously an anti-Aβ protofibril antibody at 10 mg/kg (e.g., administering BAN2401 at 10 mg/kg), biweekly, e.g., for at least 18 months or e.g., until a patient is amyloid-negative before switching to a quarterly subcutaneous maintenance dose (e.g., at a dose of 720 mg or at a dose of 360 mg).

In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a weekly subcutaneous maintenance dose (e.g., at a dose of 720 mg or at a dose of 360 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a biweekly subcutaneous maintenance dose (e.g., at a dose of 720 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a weekly subcutaneous maintenance dose (e.g., a single dose of 360 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a monthly subcutaneous maintenance dose (e.g., at a dose of 720 mg). In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every six weeks. In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every eight weeks. In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a subcutaneous maintenance dose (e.g., at a dose of 720 mg) every two months. In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., for at least 18 months or e.g., until a patient is amyloid-negative before switching to a quarterly subcutaneous maintenance dose (e.g., at a dose of 720 mg).

The following are definitions of terms used in the present application.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “about” when used in connection with doses, amounts, or ratios, include the value of a specified dose, amount, or ratio or a range of the dose, amount, or ratio that is recognized by one of ordinary skill in the art to provide a therapeutic effect equivalent to that obtained from the specified dose, amount, or ratio. The term “about” may refer to an acceptable error for a particular value as determined by one of skill in the art, which depends in part on how the values is measured or determined. In some embodiments, the term “about” means within 5% of a given value or range.

As used herein, “adjusted mean change from baseline” refers to the use of a statistical analysis to calculate the change in a biomarker value over time. In some embodiments, a linear mixed-effects model (MMRM) is used to account for at least one additional covariate to determine the adjusted mean change from baseline.

When a number is recited, either alone or as part of a numerical range, it should be understood that the numerical value can vary above and below the stated value by a variance of 10% of the stated value.

When a range of values is listed herein, it is intended to encompass each value and sub-range within that range. For example, “2.5 mg/kg to 10 mg/kg” is intended to encompass, for example, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 2.5 mg/kg to 3 mg/kg, 2.5 mg/kg to 4.5 mg/kg, 3 mg/kg to 4.5 mg/kg, 4.5 mg/kg to 8 mg/kg, 2.5 mg/kg to 9 mg/kg, and so forth.

Amyloid β 1-42 (Aβ42) refers to an amyloid beta monomer from amino acid 1 to 42 of the full-length protein (Table 5, SEQ ID NO:13). Amyloid β 1-40 (Aβ 1-40) refers to an amyloid beta monomer from amino acid 1 to 42 of the full-length protein (Table 5, SEQ ID NO:14).

Patients with “preclinical AD” or “pre-AD” as described herein, are cognitively normal individuals with intermediate or elevated levels of amyloid in the brain and can be identified by asymptomatic stages with or without memory complaints and emerging episodic memory and executive function deficits. Cognitively normal can include individuals who are CDR 0, or individuals within the normal ranges of cognitive test scores (MMSE, International Shopping List Task, Logical Memory, etc.). Preclinical AD occurs prior to significant irreversible neurodegeneration and cognitive impairment and is typically characterized by the appearance of in vivo molecular biomarkers of AD and the absence clinical symptoms. Preclinical AD biomarkers that may suggest the future development of Alzheimer's disease include, but are not limited to, one or more of intermediate or elevated levels of amyloid in the brain by amyloid or tau positron emission tomography (PET) (e.g., a centiloid measure of about 20-40, e.g., a measure of about 20-32), cerebrospinal fluid level of Aβ 1-42 and/or Aβ 1-42/1-40 ratio, cerebrospinal fluid level of total tau, cerebrospinal fluid level of neurogranin, cerebrospinal fluid level of neurofilament light peptide (NfL), and blood biomarkers as measured in the serum or plasma (e.g. levels of Aβ1-42, the ratio of two forms of amyloid-β peptide (Aβ1-42/1-40 ratio, e.g., a ratio of between about 0.092-0.094 or below about 0.092), plasma levels of plasma total tau (T-tau), levels of phosphorylated tau (P-tau) isoforms (including tau phosphorylated at 181 (P-tau181), 217 (P-tau217), and 231 (P-tau231)), glial fibrillary acidic protein (GFAP), and neurofilament light (NfL)). For example, it has been found that subjects treated with elenbecestat (E2609), a β-site amyloid precursor protein cleaving enzyme (BACE) inhibitor, who had amyloid baseline positron emission tomography (PET) standard uptake value ratios (SUVr values) of 1.4 to 1.9, exhibited the greatest slowing of cognitive decline while on treatment. See Lynch, S. Y. et al. “Elenbecestat, a BACE inhibitor: results from a Phase 2 study in subjects with mild cognitive impairment and mild-to-moderate dementia due to Alzheimer's disease.” Poster P4-389, Alzheimer's Association International Conference, Jul. 22-26, 2018, Chicago, IL, USA. Similarly, it has been found that subjects having a baseline florbetapir amyloid PET SUVr levels below 1.2 do not exhibit enough cognitive decline to be detectable, whereas subjects having SUVr levels above 1.6 appear to correlate with a plateau effect in which amyloid level has reached a saturation level and treatment does not result in a change of cognitive measures. See Dhadda, S. et al., “Baseline florbetapir amyloid PET standard update value ratio (SUVr) can predict clinical progression in prodromal Alzheimer's disease (pAD).” Poster P4-291, Alzheimer's Association International Conference, Jul. 22-26, 2018, Chicago, IL, USA.

“Early AD” or “early Alzheimer's disease,” (EAD) as used herein, is a continuum of AD severity from mild cognitive impairment due to AD-intermediate likelihood to mild Alzheimer's disease dementia. Subjects with early AD include subjects with mild Alzheimer's disease dementia as defined herein and subjects with mild cognitive impairment (MCI) due to AD-intermediate likelihood as defined herein. In some embodiments, subjects with early AD have MMSE scores of 22 to 30 and Clinical Dementia Rating (CDR) global range 0.5 to 1.0. Other methods for detecting early AD disease may employ the tests and assays specified below, including the National Institute of Aging-Alzheimer's Association (NIA-AA) core clinical criteria for probable Alzheimer's disease dementia in McKhann, G. M. et al., “The diagnosis of dementia due to Alzheimer's disease: Recommendations from the National Institute on Aging-Alzheimer's Association workgroups on diagnostic guidelines for Alzheimer's disease.” Alzheimer Dement. 2011; 7:263-9. Other methods include CDR-SB, ADCOMS Composite Clinical Score, the Mini-Mental State Examination, ADAS-Cog, ADAS MCI-ADL, modified iADRS, Wechsler Memory Scale-IV Logical Memory (subscale) I (WMS-IV LMI), and Wechsler Memory Scale-IV Logical Memory (subscale) II (WMS-IV LMII). In some embodiments, a subject with early AD has evidence of elevated amyloid in the brain or a positive amyloid load. In some embodiments, elevated amyloid in the brain or a positive amyloid load is indicated and/or confirmed by PET assessment. In some embodiments, elevated amyloid in the brain or a positive amyloid load is indicated and/or confirmed by a CSF assessment of markers such as Aβ1-42 (e.g., a soluble CSF biomarker analysis). In some embodiments, elevated amyloid in the brain or a positive amyloid load is indicated and/or confirmed by measuring the concentration of amyloid β 1-42 (Aβ42) and a concentration of amyloid β 1-40 (Aβ40) and calculating a ratio of Aβ42 to Aβ40 (Aβ42/40 ratio). In some embodiments, elevated amyloid in the brain or a positive amyloid load is indicated and/or confirmed by an MRI. In some embodiments, elevated amyloid in the brain or a positive amyloid load is indicated by retinal amyloid accumulation. In some embodiments, more than one assessment method is used.

In addition to measuring a serum or plasma Aβ1-42/1-40 ratio in a sample from a subject, the subject's amyloid level may alternatively be detected, or additionally confirmed, by one or more biomarkers such as, but not limited to: (a) amyloid detected by PET scan from either a visual read or semiquantitative thresholds (SUVr or centiloid); (b) cerebrospinal fluid (CSF) Aβ 1-42, and/or Aβ1-42/1-40 ratio; and/or (c) blood biomarkers (such as plasma Aβ1-42, tau, total tau (T-tau), and/or P-tau (e.g., P-tau181)). Secondary markers may confirm a primary amyloid determination and include but are not limited to markers of neuronal damage such as neurofilament light peptide (NfL) and markers of neuroinflammation such as glial fibrillary acidic protein (GFAP).

“Amyloid” refers to fibers that are unbranched, usually extracellular, and found in vivo; in addition, the fibers bind the dye Congo Red and then show green birefringence when viewed between crossed polarizers. Amyloid-forming proteins have been identified and associated with serious diseases, including amyloid-β peptide (Aβ) with Alzheimer's disease (AD), islet amyloid polypeptide (IAPP) with diabetes type 2, and prion protein (PrP) with the spongiform encephalopathies. As used herein, “amyloid,” “brain amyloid,” and “amyloid-β peptide (Aβ)” are used interchangeably.

In some embodiments, the subject has “elevated amyloid” or “intermediate amyloid.” As one of ordinary skill in the art will recognize, amyloid levels from amyloid PET can be reported using the Centiloid method in “centiloid” units (CL). (Klunk W E et al. The Centiloid Project: standardizing quantitative amyloid plaque estimation by PET. Alzheimer's Dement. 2015; 11:1-15 e1-4). The Centiloid method measures a tracer on a scale of 0 CL to 100 CL, where 0 is deemed the anchor-point and represents the mean in young healthy controls and 100 CL represents the mean amyloid burden present in subjects with mild to moderate severity dementia due to AD. (Id.) As is known to one of ordinary skill in the art, centiloid thresholds may vary, for example may be refined, based on new or additional scientific information. (See, e.g., http://www.gaain.org/centiloid-project.) An elevated level of amyloid can be set relative to a baseline threshold in a healthy control determined according to methods known to a POSA. For example, a centiloid value of 32.5 can be used as a threshold value for “elevated amyloid,” and an “intermediate amyloid” level refers to an Aβ amyloid PET in the range of 20-32.5 CL (e.g., 30 CL). In another example, a centiloid value of 40 can be used as a threshold value for “elevated amyloid,” and an “intermediate amyloid” level refers to an Aβ amyloid PET in the range of 20-40 CL.

Subjects with “mild Alzheimer's disease dementia,” or “mild AD dementia” as used herein, are subjects meeting the National Institute of Aging-Alzheimer's Association (NIA-AA) core clinical criteria for probable Alzheimer's disease dementia in McKhann, G. M. et al., “The diagnosis of dementia due to Alzheimer's disease: Recommendations from the National Institute on Aging-Alzheimer's Association workgroups on diagnostic guidelines for Alzheimer's disease.” Alzheimer Dement. 2011; 7:263-9. Also included herein are subjects who have a CDR score of 0.5 to 1.0 and a Memory Box score of 0.5 or greater at screening and baseline and subjects that exhibit change in the score on the Wechsler Memory Scale-Revised Logical Memory subscale II (WMS-R LM II).

Subjects with “MCI due to AD-intermediate likelihood,” as used herein are those identified as such in accordance with the NIA-AA core clinical criteria for mild cognitive impairment due to Alzheimer's disease-intermediate likelihood (see McKhann supra). For example, a subject may be symptomatic but not demented, with evidence of brain amyloid pathology making them less heterogeneous and more similar to mild Alzheimer's disease dementia subjects in cognitive and functional decline as measured by the ADCOMS Composite Clinical Score defined herein. Also included are subjects who have a CDR score of 0.5 and a Memory Box score of 0.5 or greater at screening and baseline. Furthermore, subjects who report a history of subjective memory decline with gradual onset and slow progression over the last 1 year before screening, which is corroborated by an informant, are also included herein. Memory decline and/or episodic memory impairment can be assessed in a subject by change in the score on the Wechsler Memory Scale-Revised Logical Memory subscale II (WMS-R LM II).

As used herein, “MMSE” refers to the Mini-Mental State Examination, a cognitive instrument commonly used for screening purposes, but also often measured longitudinally in AD clinical trials having a 30 point scale with higher scores indicating less impairment and lower scores indicating more impairment, ranging from 0 (most impaired) to 30 (no impairment). In some embodiments, seven items measuring orientation to time and place, registration, recall, attention, language, and drawing may be assessed as part of the MMSE score. (Folstein, M. F. et al., “Mini-mental state. A practical method for grading the cognitive state of patients for the clinician.” J. Psychiatr. Res. 1975; 12:189-98.)

As used herein, “ADAS-Cog” refers to Alzheimer's Disease Assessment Scale-Cognitive. The ADAS-Cog is a widely used cognitive scale in Alzheimer's disease trials having a structured scale that evaluates memory (word recall, delayed word recall, and word recognition), reasoning (following commands), language (naming, comprehension), orientation, ideational praxis (placing letter in envelope) and constructional praxis (copying geometric designs). (Rosen, W. G. et al., “A new rating scale for Alzheimer's disease.” Am. J. Psychiatry 1984; 141:1356-64.) Ratings of spoken language, language comprehension, word finding difficulty, ability to remember test instructions, maze, and number cancellation may also be obtained. In some embodiments, ADAS-Cog refers to the use of the Alzheimer Disease Assessment Scale-Cognitive Subscale₁₄ (ADAS-Cog14). In some embodiments, a modified version may be used herein and is scored from 0 to 90 points with a score of 0 indicating no impairment, and a score of 90 indicating maximum impairment. In some embodiments, the ADAS-Cog14 tasks include memory (word recall, delayed word recall, and word recognition), reasoning (following commands), language (naming, comprehension), orientation, ideational praxis (placing letter in envelope), constructional praxis (copying geometric designs), spoken language, language comprehension, word finding difficulty, ability to remember test instructions, maze, and number cancellation (Rosen et al, 1984).

As used herein, “CDR-SB” refers to clinical dementia rating-sum of boxes. The CDR is a clinical scale that describes 5 degrees of impairment in performance on each of 6 categories of function including memory, orientation, judgment and problem solving, community affairs, home and hobbies, and personal care. (Berg, L. et al., “Mild senile dementia of the Alzheimer type: 2. Longitudinal assessment.” Ann. Neurol. 1988; 23:477-84.) The ratings of degree of impairment obtained on each of the 6 categories of function are synthesized into 1 global rating of dementia CDR score (ranging from 0 to 3). A sum of boxes score provides an additional measure of change where each category has a maximum possible score of 3 points and the total score is a sum of the category scores giving a total possible score of 0 to 18 with higher scores indicating more impairment. The global score may be used as a clinical measure of severity of dementia.

As used herein, “ADCOMS” refers to Alzheimer's Disease Composite Score, a composite clinical score based on an analysis of four ADAS-Cog items (delayed word recall, orientation, word recognition, and word finding difficulty), two Mini Mental State Examination (MMSE) items (orientation to time, and drawing), and all six CDR-SB items (personal care, community affairs, home and hobbies, memory, orientation, and judgment and problem solving), as discussed in the Examples and in Wang, J. et al., “ADCOMS: a composite clinical outcome for prodromal Alzheimer's disease trials.” J. Neurol. Neurosurg. Psychiatry. 2016; 87:993-999. ADCOMS was developed to be particularly sensitive to disease progression during early stages of AD (i.e., preclinical AD or early AD).

In some embodiments, ADCOMS can be calculated using the following formula:

$$S ( t ) - = {\sum }_{i = 1}^{12} {a}_{i} {A}_{i} ( t ) + {\sum }_{i = 1}^{7} {b}_{i} {B}_{i} ( t ) + {\sum }_{i = 1}^{6} {c}_{i} {C}_{i} ( t )$$

where Aᵢ(t), Bᵢ(t) and Cᵢ(t) are item scores at time t corresponding to items from ADAS-cog, reversed MMSE scores, and CDR-SB, respectively (Wang, J. et al., “ADCOMS: a composite clinical outcome for prodromal Alzheimer's disease trials). ADCOMS is particularly sensitive to disease progression during early stages of AD, i.e., prodromal and mild AD.

As used herein, “ADCS MCI-ADL” refers to the Alzheimer's Disease Cooperative Study-Activities of Daily Living Scale for Mild Cognitive Impairment (ADCS MCI-ADL). The ADCS MCI-ADL is a clinical scale that assess the competence level of a patient at six basic activities of daily living. Additional examples are discussed in Kreutzer J. S., DeLuca J., Caplan B. (eds) Encyclopedia of Clinical Neuropsychology. Springer, New York, NY.

As used herein, “modified iADRS” or “iADRS” refers to a composite tool that combines scores from the ADAS Cog14 (all items) and the ADCS MCI-ADL (all items). The modified iADRS score can be used to evaluate disease progression:

$$Modified iADRS score = [ - 1 ( ADAS - cog 14 ) + 90 ] + ADCS MCI - ADL .$$

As used herein, “ApoE4-positive” subjects and “ApoE4 carriers” refer to subjects who harbor the ε4 variant of the apolipoprotein (APOE) gene. The ε4 variant is one of several major alleles of the apolipoprotein gene. The gene is generally responsible for metabolism of fats. It has been found that carriers of the apolipoprotein ε4 show significantly greater rates of amyloid retention when compared to non-carriers. (Drzezga, A. et al, “Effect of APOE genotype on amyloid plaque load and gray matter volume in Alzheimer disease.” Neurology. 2009; 72:1487-94.) In some embodiments, a subject treated herein is a heterozygous carrier of the apolipoprotein E ε4 gene allele. In some embodiments, the subject is a homozygous carrier of the apolipoprotein E ε4 gene allele. ApoE4 carriers may have a greater response to treatment when administered a composition comprising an anti-Aβ protofibril antibody (i.e. lecanemab) than ApoE4 non-carriers.

As used herein, whether an early AD subject is “amyloid positive” or “amyloid negative” may be determined based on whether the subject has a positive amyloid load. In some embodiments, a subject is determined to be amyloid-positive or amyloid-negative as indicated by longitudinal positron emission tomography (PET) assessment of an amyloid imaging agent uptake into the brain. In some embodiments, a subject is “amyloid negative” if the florbetapir amyloid PET SUVr negativity is below 1.17. In some embodiments, a subject is determined to be amyloid-positive or amyloid-negative by evaluation of the Aβ42/40 ratio in a sample (e.g., a plasma sample) from a subject, alone or in combination with another method such as PET measurement of brain amyloid. In some embodiments, a subject is “amyloid negative” if the Aβ42/40 ratio in a sample is at or about above 0.092-0.094 e.g., at about 0.092. In some embodiments, a subject is “amyloid negative” if the Aβ42/40 ratio in a sample is above 0.092. In some embodiments, a subject is determined to be amyloid-positive or amyloid-negative by a CSF assessment of the presence of amyloid pathology using assessments of markers such as Aβ1-42 (e.g., a soluble CSF biomarker analysis), alone or in combination with another method such as PET measurement of brain amyloid. In some embodiments, a qualitative visual read of PET scans may be used to determine amyloid positive and amyloid negative by categorizing subjects as having either “normal” or “abnormal” uptake on the basis of the PET image pattern. Readers will have been trained and certified to recognize brain PET images with abnormal or normal patterns of uptake, or the detection of amyloid is done through a semi-quantitative or quantitative approach. In some embodiments, a threshold will be set for quantitatively determining from a biomarker (e.g., serum or CSF) and/or PET scan whether an Aβ brain load indicates a subject is amyloid-positive or negative. In some embodiments, a subject is determined to be amyloid-positive or amyloid-negative by an MRI. In some embodiments, a subject is determined to be amyloid-positive or amyloid-negative by retinal amyloid accumulation. In some embodiments, a subject is determined to be amyloid-positive or amyloid-negative by behavioral/cognitive phenotypes.

As would be understood by one of ordinary skill in the art, digital, computerized, and/or conventional (e.g., pen and paper) cognitive tests may be used to detect early cognitive changes that may signal mild cognitive impairment and/or a risk for developing dementia, and thus may be used to identify subject in need of treatment as disclosed herein. Such tests, for example, may screen for cognitive impairment, and potentially identify individuals with MCI. Tests may use artificial intelligence to analyze cognitive test results to determine whether a case of mild cognitive impairment will escalate into Alzheimer's within a year. Diagnosing the condition early, before symptoms have begun to appear, may be used to assist physicians identify subjects in need of treatment as disclosed herein sooner, potentially delaying onset or lessening the severity of the neurodegenerative disease.

As used herein, the term “treat” refers to any administration or application of a therapeutic agent for a disease or disorder in a subject, and includes inhibiting the disease, slowing progression of the disease, delaying progression, arresting its development, reversing progression of disease (e.g., reversing build up of Aβ fibrils), preventing the onset or development of the disease, relieving or ameliorating one or more symptoms or underlying condition(s) of the disease, curing the disease, improving one or more clinical metrics, or preventing reoccurrence of one or more symptoms of the disease. In some embodiments, treatment of AD in a subject comprises an administration, e.g., an intravenous infusion, of an anti-amyloid β (Aβ) protofibril antibody.

As used herein, the term “infusion” refers to an active administration of one or more agents with an infusion time of, for example, approximately 60 minutes. In some embodiments, an anti-amyloid β (Aβ) protofibril antibody, described herein is systemically administered to a human subject via infusion. In some embodiments, an anti-amyloid β (Aβ) protofibril antibody is alternatively administered to the human subject, e.g., by subcutaneous injection. In some embodiments, the subcutaneous injection is a weekly injection. In some embodiments, the subcutaneous injection is a biweekly injection. In some embodiments, an anti-amyloid β (Aβ) protofibril antibody is administered to the human subject by intravenous infusion.

In some embodiments, the subject is administered a maintenance dose of a treatment. As used herein, the term “maintenance dose” refers to a dosage administered to a subject to maintain the desired therapeutic effect. In some embodiments, the maintenance dose is administered weekly, every two weeks, monthly, every two months, or every three months (quarterly) or every 24 weeks (every six months or semi-annually). In some embodiments, the maintenance dose comprises an anti-Aβ protofibril antibody. In some embodiments, the maintenance dose is administered as an intravenous infusion. In some embodiments, the intravenous infusion is administered biweekly (Q2W). In some embodiments, the intravenous infusion is administered every 4 weeks (Q4W). In some embodiments, the intravenous infusion is administered every 3 months (Q3M). In some embodiments, the intravenous infusion is a 10 mg/kg dose of BAN2401. In some embodiments, the intravenous infusion is a 10 mg/kg dose of BAN2401 administered biweekly. In some embodiments, the maintenance dose is administered subcutaneously, orally, or nasally. In some embodiments, the maintenance dose is administered subcutaneously.

In some embodiments, the maintenance dose is administered as a subcutaneous injection. In some embodiments, the maintenance dose is administered as a weekly, subcutaneous injection. In some embodiments, the maintenance dose is administered as a biweekly, subcutaneous injection. In some embodiments, the maintenance dose is administered as a monthly, subcutaneous injection. In some embodiments, the maintenance dose is administered as a quarterly, subcutaneous injection. In some embodiments, the maintenance dose is administered weekly or less frequently, e.g., every two weeks (biweekly), every four weeks, monthly, every six weeks, every eight weeks (2 months), every three months (quarterly) or every six monthly (semi-annually). In some embodiments, the maintenance dose is provided in a single administration, e.g., administered as a single subcutaneous injection of 720 or 1440 mg, or in two or more administrations, e.g., two concurrent administrations of 360 mg for a total of 720 mg or two administrations of 720 mg for a total of 1440 mg or four administrations of 360 mg for a total of 1440 mg. In some embodiments, the maintenance dose is 120 mg. In some embodiments, the maintenance dose is 180 mg. In some embodiments, the maintenance dose is 240 mg. In some embodiments, the maintenance dose is 360 mg. In some embodiments, the maintenance dose is 440 mg. In some embodiments, the maintenance dose is 480 mg. In some embodiments, the maintenance dose is 540 mg. In some embodiments, the maintenance dose is 440 mg. In some embodiments, the maintenance dose is 580 mg. In some embodiments, the maintenance dose is 600 mg. In some embodiments, the maintenance dose is 720 mg. In some embodiments, the maintenance dose is 840 mg. In some embodiments, the maintenance dose is 900 mg. In some embodiments, the maintenance dose is 960 mg. In some embodiments, the maintenance dose is 1080 mg. In some embodiments, the maintenance dose is 1200 mg. In some embodiments, the maintenance dose is 1260 mg. In some embodiments, the maintenance dose is 1320 mg. In some embodiments, the maintenance dose is 1440 mg. In some embodiments, the maintenance dose is administered as a weekly, subcutaneous injection of 720 mg. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 720 mg. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a biweekly, subcutaneous injection of 1440 mg. In some embodiments, the maintenance dose is provided in a single, biweekly administration of 1440 mg comprising two concurrent, e.g., two sequential administrations of 720 mg of the subcutaneous formulation for a total of 1440 mg or four sequential administrations of 360 mg for a total of 1440 mg.

In some embodiments, the maintenance dose is administered once or multiple times. In some embodiments, the maintenance dose is administered at a lower dose than during an earlier course of treatment and/or is administered less frequently than during the earlier course of treatment.

In some embodiments, after switching to a maintenance dose, a subject's biomarker levels may indicate increasing levels of amyloid in the brain. In some embodiments, after switching to a maintenance dose, a subject's biomarker levels, e.g. the plasma Aβ42/40 ratio, may began to decrease, indicating increasing levels of amyloid in the brain. In some embodiments, a subject on a maintenance dose may have a decrease in the Aβ42/40 ratio. In some embodiments, a subject is put on a maintenance dose chosen such that the subject may have a decrease in the Aβ42/40 ratio but the Aβ42/40 ratio may remain above the threshold for amyloid positivity, e.g. for at least one year (e.g., at least 1, 2, 3, 4, 5, 6, 7, 8, 9, or 10 years).

In some embodiments, after switching to a maintenance dose, a subject's biomarker levels, e.g. p-tau181, may began to increase, indicating increasing levels of amyloid in the brain. In some embodiments, a subject on a maintenance dose may have an increase in plasma p-tau181. In some embodiments, a subject on a maintenance dose may have an increase in p-tau181 but the level p-tau181 may remain below the threshold for amyloid positivity, e.g., for at least one year (e.g., at least 1, 2, 3, 4, 5, 6, 7, 8, 9, or 10 years).

As used herein, the term “prevent” refers to obtaining beneficial or desired results including, but not limited to, prophylactic benefit. For prophylactic benefit, the composition may be administered to a subject at risk of developing Alzheimer's disease, to a subject having one or more preclinical symptoms but not clinical symptoms of Alzheimer's disease, or to a subject reporting one or more of the physiological symptoms of Alzheimer's disease, even though a clinical diagnosis of having Alzheimer's has not been made. As used herein “prevention” may further include therapeutic benefit, by which is meant eradication or amelioration of the underlying condition being treated or of one or more of the physiological symptoms associated therewith.

As used herein, the term “ARIA” refers to amyloid-related imaging abnormality as evaluated using MRI. In some embodiments, ARIA includes amyloid related imaging abnormality edema/effusion (ARIA-E). In some embodiments, ARIA includes amyloid related imaging abnormality hemorrhage (ARIA-H). In some embodiments, subjects with ARIA experience headache, confusion, and/or seizure and these may be used to identify a subject with ARIA or to indicate further evaluation for ARIA. In some embodiments, ARIA is evaluated at specified intervals during treatment. In some embodiments, ARIA is evaluated when the subject experiences symptoms of ARIA. In some embodiments, maximum serum concentration (Cmax) of anti-A protofibril antibody can be used as a predictor of the risk of ARIA-E. In some embodiments, the use of a subcutaneous formulation may provide a reduced risk of ARIA-E (e.g., due to a lower Cmax) compared to an IV administration.

As used herein, the term “clinical decline” refers to a worsening of one or more clinical symptoms of AD. Methods for measuring clinical decline may employ the tests and assays specified herein. In some embodiments, clinical decline is determined by a worsening of ADCOMS. In some embodiments, clinical decline is determined by a worsening of MMSE. In some embodiments, clinical decline is determined by a worsening of ADAS-Cog. In some embodiments, clinical decline is determined by a worsening of FAQ. In some embodiments, clinical decline is determined by a worsening of CDR-SB. In some embodiments, clinical decline is determined by a worsening of Wechsler Memory Scale-IV Logical Memory (subscale) I and/or (subscale) II. In some embodiments, clinical decline is determined by a worsening of CDR score. In some embodiments, clinical decline refers to a worsening in one or more biomarkers of AD or brain measurement (e.g., by PET or MRI), e.g., of brain atrophy and/or amyloid accumulation.

As used herein, the term “blood sample” or “blood” refers to a sample of blood, including serum and/or blood plasma from a human subject. In some embodiments, blood will be collected from subjects to evaluate potential biomarkers of AD that may include amyloid fragments and isoforms, tau, and other protein biomarkers (e.g., NFL) for association with AD diagnosis, amyloid or tau load, or disease modification. In some embodiments, subjects are required to fast if possible before collection at Week 96 and Week 216. In other embodiments and/or at other time points, subjects do not require fasting. Pre-AD biomarker levels that may suggest the development of Alzheimer's disease include, but are not limited to, brain amyloid level, cerebrospinal fluid level of Aβ1-42, cerebrospinal fluid level of total tau, cerebrospinal fluid level of neurogranin, and cerebrospinal fluid level of neurofilament light chain (NfL).

### Measurement of Aβ42/40 Ratio

The disclosure and methods discussed herein depend in part on the surprising discovery that not only can Aβ42 and 40 be measured to calculate a ratio in blood samples, but also that treatment comprising an anti-Aβ protofibril antibody such as BAN2401 can lead to an increase in the ratio that correlates with reduced brain amyloid load and improved cognitive outcomes in subjects. The change in the ratio can therefore be used, in various embodiments, as a less invasive measure of treatment efficacy and to allow for monitoring and treatment decisions such as whether to increase or decrease the amount of antibody being administered, whether to increase or decrease the frequency of administration, whether to introduce a further therapeutic agent, and/or whether to discontinue treatment with the anti-Aβ protofibril antibody.

Methods for measuring the Aβ42/40 ratio are known in the art, such as assays using LC MS/MS. Methods may include the PrecivityAD™ assay (see, e.g., Kirmess et al., J. Clinica Chimica Acta 519: 267-275 (2021)) and the Sysmex assay (https://www.eisai.com/news/2019/news201990.html) for measuring Aβ42 and Aβ40 in a sample to use in calculating a ratio.

The measurement of the Aβ42/40 ratio may be used alone to evaluate treatment efficacy, or in conjunction with one or more additional criteria, such as PET measurement of Aβ radiotracer update, MRI evaluation of Aβ plaque, and/or behavioral measures, as discussed herein. Such assays may also be used to diagnose patients eligible for treatment (e.g. by measuring an Aβ42/40 ratio and determining a subject is suitable for treatment because of a lower ratio than observed in a healthy control subject, alone or in conjunction with measuring one or more additional marker of AD pathology in the subject). In some embodiments, the measurement of an Aβ42/40 ratio may be used in place of another method of measuring brain amyloid levels, such as a PET scan for determining a subject is suitable for treatment. In some embodiments, the measurement of an Aβ42/40 ratio may be used in place of another method of measuring brain amyloid levels, such as a PET scan for determining treatment efficacy and/or making treatment decisions such as whether to continue treatment, switch to a maintenance dose, etc.

In some embodiments, an Aβ42/40 ratio measurement may employ a relative change from baseline measurement. In some embodiments, an Aβ42/40 ratio measurement may employ a set threshold to determine a change in brain amyloid levels, e.g., to identify a patient suitable for treatment, e.g., with an anti-Aβ protofibril antibody, or to determine whether to continue treatment, or to determine whether to switch to a maintenance dose, or to conclude a patient is amyloid negative. In some embodiments, the threshold may be evaluated in conjunction with another measurement of brain amyloid load, such as a PET scan, to assist in determining whether a subject is suitable for treatment or continued treatment. In some embodiments, an Aβ42/40 ratio threshold may be used in place of another method of measuring brain amyloid levels, such as a PET scan. In some embodiments, an Aβ42/40 ratio threshold is at or about 0.09, 0.091, 0.092, 0.093, 0.094, 0.095, 0.096, 0.097, 0.099, 0.1. In some embodiments, the threshold is about 0.092. In some embodiments, the threshold is 0.092. In some embodiments, the threshold is about 0.094. In some embodiments, a decrease in the Aβ42/40 ratio below a threshold value may indicate a need to continue treatment or to select an increase in a dosing regimen. In some embodiments, an increase in the Aβ42/40 ratio above a threshold value may be used to indicate a treatment may be terminated (e.g., terminated in favor of a maintenance regimen) and/or to otherwise to determine a decrease in a dosing regimen or discontinuation in treatment. In some embodiments, a decrease in the Aβ42/40 ratio below a threshold value may be used to determine whether to discontinue a maintenance dosing regimen, e.g., and return to the prior treatment regimen.

### Anti-Aβ Protofibril Antibodies

In some embodiments, any anti-Aβ protofibril antibody may be used in the methods disclosed herein. In some embodiments, the antibody comprises one or more of the sequences listed in Tables 1-4, e.g., comprising a complete set of 6 complementarity determining regions (CDRs) and/or a complete set of variable regions and/or a complete set of heavy and light chain sequences from the tables. In some embodiments, the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3). In some embodiments, the anti-Aβ protofibril antibody comprises a heavy chain variable region comprising an amino acid sequence of SEQ ID NO: 7 and a light chain variable region comprising an amino acid sequence of SEQ ID NO: 8. In some embodiments, the anti-Aβ protofibril antibody comprises a heavy chain comprising an amino acid sequence of SEQ ID NO: 9 and a light chain comprising an amino acid sequence of SEQ ID NO: 10. “CDRs” used herein in the context of an antibody sequence or structure refers to complementarity determining regions, that provide the main determinants of antigen binding. Generally, the antigen-binding site has six CDRs; three in the VH (HCDR1, HCDR2, HCDR3), and three in the VL (LCDR1, LCDR2, LCDR3). The CDRs may be determined according to the Kabat numbering scheme. which may be determined by according to the Kabat numbering scheme (Kabat et al., Sequences of Proteins of Immunological Interest, 5th Ed. Public Health Service, National Institutes of Health, Bethesda, Md., 1991, hereafter referred to as “Kabat report”).

In some embodiments, the at least one anti-Aβ protofibril antibody comprises a human constant region. In some embodiments, the human constant region of the at least one anti-Aβ protofibril antibody comprises a heavy chain constant region chosen from IgG1, IgG2, IgG3, IgG4, IgM, IgA, IgE, and any allelic variation thereof as disclosed in the Kabat report. Any one or more of such sequences may be used in the present disclosure. In some embodiments, the heavy chain constant region is chosen from IgG1 and allelic variations thereof. The amino acid sequence of human IgG1 constant region is known in the art and set out in SEQ ID NO: 11.

In some embodiments, the human constant region of the at least one anti-Aβ antibody comprises a light chain constant region chosen from λ-α-chain constant regions and any allelic variation thereof as discussed in the Kabat report. Any one or more of such sequences may be used in the present disclosure. In some embodiments, the light chain constant region is chosen from κ and allelic variations thereof. The amino acid sequence of human κ chain constant region is known in the art and set out in SEQ ID NO: 12.

In some embodiments, the at least one anti-Aβ protofibril antibody comprises human heavy and light chain variable region frameworks. In some embodiments, the at least one anti-Aβ protofibril antibody comprises a heavy chain variable region comprising an amino acid sequence of SEQ ID NO: 7, and a light chain variable region comprising an amino acid sequence of SEQ ID NO: 8. In some embodiments, the at least one anti-Aβ protofibril antibody comprises a human IgG1 heavy chain constant region, and a human Ig kappa light chain constant region. In some embodiments, the at least one anti-Aβ protofibril antibody comprises a heavy chain constant region comprising an amino acid sequence of SEQ ID NO: 11, and a light chain constant region comprising an amino acid sequence of SEQ ID NO: 12.

In some embodiments, the at least one anti-Aβ protofibril antibody is BAN2401, also known as lecanemab. The terms “BAN2401” and “lecanemab” are used interchangeably and refer to a humanized IgG1 monoclonal version of mAb158, which is a murine monoclonal antibody raised to target protofibrils and disclosed in WO 2007/108756 and Journal of Alzheimer's Disease 43: 575-588 (2015). BAN2401 comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3) and is described in WO 2007/108756 and in Journal of Alzheimer's Disease 43:575-588 (2015). BAN2401 comprises (i) a heavy chain variable region comprising the amino acid sequence of SEQ ID NO: 7 and (ii) a light chain variable region comprising the amino acid sequence of SEQ ID NO: 8. The full length sequences of heavy chain and light chain of BAN2401 are set forth in SEQ ID NOs: 9 and 10 and is described in WO 2007/108756 and in Journal of Alzheimer's Disease 43:575-588 (2015).

Other non-limiting examples of suitable antibodies for use as the at least one anti-Aβ protofibril antibody in the present disclosure include aducanumab, as well as those disclosed in WO 2002/003911, WO 2005/123775, WO 2007/108756, WO 2011/001366, WO 2011/104696, and WO 2016/005466.

In some embodiments, the isolated anti-Aβ protofibril antibody is present in a concentration of at least 80 mg/mL. In some embodiments, the isolated anti-Aβ protofibril antibody is present in a concentration of at least 100 mg/mL. In some embodiments, the isolated anti-Aβ protofibril antibody is present in a concentration of at least 200 mg/mL. In some embodiments, the isolated anti-Aβ protofibril antibody is present in a concentration of at least 250 mg/mL. In some embodiments, the isolated antibody or fragment thereof is present in a concentration ranging from 80 mg/mL to 300 mg/mL. In some embodiments, the isolated anti-Aβ protofibril antibody is present in a concentration ranging from 85 mg/mL to 275 mg/mL. In some embodiments, the isolated anti-Aβ protofibril antibody is present in a concentration ranging from 90 mg/mL to 250 mg/mL. In some embodiments, the isolated anti-Aβ protofibril antibody is present in a concentration ranging from 95 mg/mL to 225 mg/mL. In some embodiments, the isolated anti-Aβ protofibril antibody is present in a concentration ranging from 100 mg/mL to 200 mg/mL. In some embodiments, the isolated antibody or fragment thereof is present in a concentration of 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, or 300 mg/mL. In some embodiments, the isolated antibody or fragment thereof is present in a concentration of 100 mg/mL. In some embodiments, the isolated antibody or fragment thereof is present in a concentration of 200 mg/mL. In some embodiments, the isolated antibody or fragment thereof is present in a concentration of 250 mg/mL. In some embodiments, the isolated antibody or fragment thereof is present in a concentration of 300 mg/mL. In some embodiments, the isolated antibody or fragment thereof is BAN2401.

As used herein, a “fragment” of an antibody comprises a portion of the antibody, for example comprising an antigen-binding or a variable region thereof. Non-limiting examples of fragments include Fab fragments, Fab′ fragments, F(ab′)2 fragments, Fv fragments, diabodies, linear antibodies, and single-chain antibody molecules.

### Therapeutically Effective Amount of at Least One Anti-Aβ Protofibril Antibody

In various embodiments, the methods of the present disclosure comprise administering to a subject a composition comprising a therapeutically effective amount of at least one anti-Aβ protofibril antibody. As used herein, the term a “therapeutically effective amount” refers to an amount of a compound or pharmaceutical composition sufficient to produce a desired therapeutic effect. In various embodiments, the therapeutically effective amount is an amount sufficient to increase an Aβ42/40 ratio when comparing the ratio in a sample, e.g., a blood sample, before and after treatment. In some embodiments, the therapeutically effective amount is initially 2.5-15 mg/kg, e.g., about 10 mg/kg. In some embodiments, after administering a first therapeutically effective amount for a period of time, e.g., 6-12 months or more, a second therapeutically effective amount is administered at a lower dosage, e.g., if an increase in an Aβ42/40 ratio is observed before and after administering the first therapeutically effective amount. In some embodiments, the second therapeutically effective amount is accompanied by one or more additional therapy, e.g., a BACE inhibitor and/or anti-tau antibody therapy. In some embodiments, the at least one additional therapeutic agent comprises one or more of BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof. In some embodiments, the at least one additional therapeutic agent comprises a BACE inhibitor. In some embodiments, the BACE inhibitor is chosen from CNP520, BI-1181181, LY2886721, LY3202626, PF-06751979, RG7129, atabecestat, elenbecestat, lanabecestat, and verubecestat. In some embodiments, the BACE inhibitor is elenbecestat.

One of ordinary skill in the art will understand that the therapeutically effective amount of the at least one anti-Aβ protofibril antibody administered to a subject may depend upon a number of factors including pharmacodynamic characteristics, route of administration, frequency of treatment, and health, age, and weight of the subject to be treated and, with the information disclosed herein, will be able to determine the appropriate amount for each subject.

In some embodiments, the therapeutically effective amount is a dose chosen to improve efficacy and/or maintain efficacy and improve at least one of safety and tolerability. In some embodiments, the therapeutically effective amount is chosen to lower at least one side effect and simultaneously improve efficacy and/or maintain efficacy.

In some embodiments, 0.5 mg/kg to 45 mg/kg, 0.5 mg/kg to 40 mg/kg, 0.5 mg/kg to 35 mg/kg, 0.5 mg/kg to 30 mg/kg, 0.5 mg/kg to 25 mg/kg, 0.5 mg/kg to 20 mg/kg, 0.5 mg/kg to 15 mg/kg, 0.5 mg/kg to 10 mg/kg, 0.5 mg/kg to 5 mg/kg, or 0.5 mg/kg to 2.5 mg/kg of at least one anti-Aβ protofibril antibody is administered to the subject relative to body weight of the subject.

In some embodiments, 2.5 mg/kg to 45 mg/kg, 2.5 mg/kg to 40 mg/kg, 2.5 mg/kg to 35 mg/kg, 2.5 mg/kg to 30 mg/kg, 2.5 mg/kg to 25 mg/kg, 2.5 mg/kg to 20 mg/kg, 2.5 mg/kg to 15 mg/kg, 2.5 mg/kg to 10 mg/kg, or 2.5 mg/kg to 5 mg/kg of at least one anti-Aβ protofibril antibody is administered to the subject relative to body weight of the subject.

In some embodiments, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, or 5 mg/kg to 10 mg/kg, of at least one anti-Aβ protofibril antibody is administered to the subject relative to body weight of the subject.

In some embodiments, 7.5 mg/kg to 45 mg/kg, 7.5 mg/kg to 40 mg/kg, 7.5 mg/kg to 35 mg/kg, 7.5 mg/kg to 30 mg/kg, 7.5 mg/kg to 25 mg/kg, 7.5 mg/kg to 20 mg/kg, 7.5 mg/kg to 15 mg/kg, or 7.5 mg/kg to 10 mg/kg of at least one anti-Aβ protofibril antibody is administered to the subject relative to body weight of the subject.

In some embodiments, from 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg of at least one anti-Aβ protofibril antibody is administered to the subject relative to body weight of the subject. In some embodiments, up to 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, or 0.5 mg/kg of at least one anti-Aβ protofibril antibody is administered to the subject relative to body weight of the subject.

In some embodiments, 0.5 mg/kg of at least one anti-Aβ protofibril antibody is administered to the subject relative to body weight of the subject. In some embodiments, 1 mg/kg of at least one anti-Aβ protofibril antibody is administered to the subject relative to body weight of the subject. In some embodiments, 2 mg/kg of at least one anti-Aβ protofibril antibody is administered to the subject relative to body weight of the subject. In some embodiments, 2.5 mg/kg of at least one anti-Aβ protofibril antibody is administered to the subject relative to body weight of the subject. In some embodiments, 3 mg/kg of at least one anti-Aβ protofibril antibody is administered to the subject relative to body weight of the subject. In some embodiments, 4 mg/kg of at least one anti-Aβ protofibril antibody is administered to the subject relative to body weight of the subject. In some embodiments, 5 mg/kg of at least one anti-Aβ protofibril antibody is administered to the subject relative to body weight of the subject. In some embodiments, 6 mg/kg of at least one anti-Aβ protofibril antibody is administered to the subject relative to body weight of the subject. In some embodiments, 7 mg/kg of at least one anti-Aβ protofibril antibody is administered to the subject relative to body weight of the subject. In some embodiments, 7.5 mg/kg of at least one anti-Aβ protofibril antibody is administered to the subject relative to body weight of the subject. In some embodiments, 8 mg/kg of at least one anti-Aβ protofibril antibody is administered to the subject relative to body weight of the subject. In some embodiments, 9 mg/kg of at least one anti-Aβ protofibril antibody is administered to the subject relative to body weight of the subject. In some embodiments, 10 mg/kg of at least one anti-Aβ protofibril antibody is administered to the subject relative to body weight of the subject. In some embodiments, 11 mg/kg of at least one anti-Aβ protofibril antibody is administered to the subject relative to body weight of the subject. In some embodiments, 12 mg/kg of at least one anti-Aβ protofibril antibody is administered to the subject relative to body weight of the subject. In some embodiments, 12.5 mg/kg of at least one anti-Aβ protofibril antibody is administered to the subject relative to body weight of the subject. In some embodiments, 13 mg/kg of at least one anti-Aβ protofibril antibody is administered to the subject relative to body weight of the subject. In some embodiments, 14 mg/kg of at least one anti-Aβ protofibril antibody is administered to the subject relative to body weight of the subject. In some embodiments, 15 mg/kg of at least one anti-Aβ protofibril antibody is administered to the subject relative to body weight of the subject. In some embodiments, 16, 17, 18, 19, or 20 mg/kg of at least one anti-Aβ protofibril antibody is administered to the subject relative to body weight of the subject. In some embodiments, 21, 22, 23, 24, or 25 mg/kg of at least one anti-Aβ protofibril antibody is administered to the subject relative to body weight of the subject.

In some embodiments, 27.5 mg/kg, 30 mg/kg, 32.5 mg/kg, 35 mg/kg, 37.5 mg/kg, 40 mg/kg, 42.5 mg/kg, 45 mg/kg, 47.5 mg/kg, or 50 mg/kg of at least one anti-Aβ protofibril antibody is administered to the subject relative to body weight of the subject.

As mentioned, in some embodiments, the at least one anti-Aβ protofibril antibody is BAN2401. Accordingly, in some embodiments, 0.5 mg/kg to 45 mg/kg, 0.5 mg/kg to 40 mg/kg, 0.5 mg/kg to 35 mg/kg, 0.5 mg/kg to 30 mg/kg, 0.5 mg/kg to 25 mg/kg, 0.5 mg/kg to 20 mg/kg, 0.5 mg/kg to 15 mg/kg, 0.5 mg/kg to 10 mg/kg, 0.5 mg/kg to 5 mg/kg, or 0.5 mg/kg to 2.5 mg/kg of BAN2401 is administered to the subject relative to body weight of the subject.

In some embodiments, 2.5 mg/kg to 45 mg/kg, 2.5 mg/kg to 40 mg/kg, 2.5 mg/kg to 35 mg/kg, 2.5 mg/kg to 30 mg/kg, 2.5 mg/kg to 25 mg/kg, 2.5 mg/kg to 20 mg/kg, 2.5 mg/kg to 15 mg/kg, 2.5 mg/kg to 10 mg/kg, or 2.5 mg/kg to 5 mg/kg of BAN2401 is administered to the subject relative to body weight of the subject.

In some embodiments, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, or 5 mg/kg to 10 mg/kg of BAN2401 is administered to the subject relative to body weight of the subject.

In some embodiments, 7.5 mg/kg to 45 mg/kg, 7.5 mg/kg to 40 mg/kg, 7.5 mg/kg to 35 mg/kg, 7.5 mg/kg to 30 mg/kg, 7.5 mg/kg to 25 mg/kg, 7.5 mg/kg to 20 mg/kg, 7.5 mg/kg to 15 mg/kg, or 7.5 mg/kg to 10 mg/kg of BAN2401 is administered to the subject relative to body weight of the subject.

In some embodiments, from 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg of BAN2401 is administered to the subject relative to body weight of the subject. In some embodiments, up to 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, or 0.5 mg/kg of BAN2401 is administered to the subject relative to body weight of the subject.

In some embodiments, 0.5 mg/kg of BAN2401 is administered to the subject relative to body weight of the subject. In some embodiments, 1 mg/kg of BAN2401 is administered to the subject relative to body weight of the subject. In some embodiments, 2 mg/kg of BAN2401 is administered to the subject relative to body weight of the subject. In some embodiments, 2.5 mg/kg of BAN2401 is administered to the subject relative to body weight of the subject. In some embodiments, 3 mg/kg of BAN2401 is administered to the subject relative to body weight of the subject. In some embodiments, 4 mg/kg of BAN2401 is administered to the subject relative to body weight of the subject. In some embodiments, 5 mg/kg of BAN2401 is administered to the subject relative to body weight of the subject. In some embodiments, 6 mg/kg of BAN2401 is administered to the subject relative to body weight of the subject. In some embodiments, 7 mg/kg of BAN2401 is administered to the subject relative to body weight of the subject. In some embodiments, 7.5 mg/kg of BAN2401 is administered to the subject relative to body weight of the subject. In some embodiments, 8 mg/kg of BAN2401 is administered to the subject relative to body weight of the subject. In some embodiments, 9 mg/kg of BAN2401 is administered to the subject relative to body weight of the subject. In some embodiments, 10 mg/kg of BAN2401 is administered to the subject relative to body weight of the subject. In some embodiments, 11 mg/kg of BAN2401 is administered to the subject relative to body weight of the subject. In some embodiments, 12 mg/kg of BAN2401 is administered to the subject relative to body weight of the subject. In some embodiments, 12.5 mg/kg of BAN2401 is administered to the subject relative to body weight of the subject. In some embodiments, 13 mg/kg of BAN2401 is administered to the subject relative to body weight of the subject. In some embodiments, 14 mg/kg of BAN2401 is administered to the subject relative to body weight of the subject. In some embodiments, 15 mg/kg of BAN2401 is administered to the subject relative to body weight of the subject. In some embodiments, 16, 17, 18, 19, or 20 mg/kg of BAN2401 is administered to the subject relative to body weight of the subject. In some embodiments, 21, 22, 23, 24, or 25 mg/kg of BAN2401 is administered to the subject relative to body weight of the subject. In some embodiments, 27.5 mg/kg, 30 mg/kg, 32.5 mg/kg, 35 mg/kg, 37.5 mg/kg, 40 mg/kg, 42.5 mg/kg, 45 mg/kg, 47.5 mg/kg, or 50 mg/kg of BAN2401 is administered to the subject relative to body weight of the subject.

In some embodiments, 2.5 mg/kg to 10 mg/kg of BAN2401 is administered to the subject relative to body weight of the subject. In some embodiments, 5 mg/kg to 10 mg/kg of BAN2401 is administered to the subject relative to body weight of the subject. In some embodiments, 2.5 mg/kg of BAN2401 is administered to the subject relative to body weight of the subject. In some embodiments, 5 mg/kg of BAN2401 is administered to the subject relative to body weight of the subject. In some embodiments, 7.5 mg/kg of BAN2401 is administered to the subject relative to body weight of the subject. In some embodiments, 10 mg/kg of BAN2401 is administered to the subject relative to body weight of the subject. In some embodiments, a reduced concentration of BAN2401 is administered if the initial dosing increases an Aβ42/40 ratio when comparing the ratio in a sample, e.g., a blood sample, before and after the initial treatment.

In some embodiments, a subject is administered a first dose of the anti-Aβ protofibril antibody without an initial titrating step up to the treatment dose (e.g., a subject starts treatment at 10 mg/kg with no titration). In some embodiments, a dose of BAN2401 may be used in treating AD without the need of a prior titrating step. In some embodiments, a subject is switched to a maintenance dose without an initial titrating step to the maintenance dose. Without being bound by theory, providing a therapeutic dose without a titration step may provide additional therapeutic benefits to the patient, e.g., a faster shift in plasma biomarkers toward amyloid negativity or facilitating identification sooner of patients that do not have a therapeutic change in plasma biomarkers in response to the anti-Aβ protofibril antibody (non-responders) and who would benefit from alternative treatment.

### Dosing Regimens for at Least One Anti-Aβ Protofibril Antibody

In various embodiments, the methods of the present disclosure comprise administering to a subject a composition comprising a therapeutically effective amount of at least one anti-Aβ protofibril antibody at a schedule that may be fixed and/or adjusted over time. One of ordinary skill in the art will understand that any of the therapeutically effective amounts of the at least one anti-Aβ protofibril antibody disclosed above may be administered one or more times according to one or more dosing regimens. One of ordinary skill in the art will be able to determine, depending upon a number of factors including pharmacodynamic characteristics, route of administration, dose, and health, age, and weight of the subject to be treated and, with the information disclosed herein, the appropriate dosing regimen(s) for each subject.

In some embodiments, a composition comprising, e.g., 2.5-15 mg/kg, e.g., 10 mg/kg of at least one anti-Aβ protofibril antibody relative to body weight of the subject, is administered to the subject once every two to four weeks. In some embodiments, the composition is administered to the subject once every month. In various embodiments, the composition is administered, e.g., biweekly or monthly, to increase an Aβ42/40 ratio when comparing the ratio in a sample, e.g., a blood sample, before and after treatment. In some embodiments, after administering a first composition, e.g., biweekly or monthly for a period of time, e.g., 6-12 months or more, a reduced dosing frequency is used, e.g., every 3, 4, 5, 6, 7, or 8 weeks, or every 2, 3, 4, 5, 6, 7, 8, 9, 10, 11, or 12 months, or more, if an increase in an Aβ42/40 ratio is observed. In some embodiments, the reduced frequency is accompanied by one or more additional therapy, e.g., a BACE inhibitor and/or anti-tau antibody therapy.

In some embodiments, a composition comprising at least one anti-Aβ protofibril antibody is administered every day, every other day, every third day, once every week, once every two weeks (“bi-weekly”, “biweekly” or “bw”), once every three weeks, once every four weeks (“four-week interval”), once every month (“mo”), once every five weeks, once every six weeks, once every seven weeks, once every eight weeks, once every two months, once every nine weeks, once every ten weeks, once every eleven weeks, once every twelve weeks, once every three months (quarterly), once every fourteen weeks, once every sixteen weeks, once every four months, once every eighteen weeks, once every twenty weeks, once every five months, once every 22 weeks, once every 24 weeks, once every six months (semi-annually), once every seven months, once every eight months, once every nine months, once every ten months, once every eleven months, once every twelve months (annually), once every thirteen months, once every fourteen months, once every fifteen months, once every sixteen months, once every seventeen months, or once every eighteen months. In some embodiments, a composition comprising at least one anti-Aβ protofibril antibody is administered every day, every other day, every third day, once every week, once every two weeks (“biweekly”), once every four weeks (“four-week interval”), or once every month. In some embodiments, a composition comprising a therapeutically effective amount of at least one anti-Aβ protofibril antibody is administered once every two weeks or once every four weeks. In some embodiments, a composition comprising a therapeutically effective amount of at least one anti-Aβ protofibril antibody is administered once every two weeks. In some embodiments, a composition comprising a therapeutically effective amount of at least one anti-Aβ protofibril antibody is administered once every four weeks.

In some embodiments, the initial treatment is administered for 1, 2, 3, 4, 5, 6, 7, 8, 9, 10, 11, 12, 13, 14, 15, 16, 17, 18, or more months. In some embodiments, before and after the initial treatment, an Aβ42/40 ratio is measured in a sample, e.g., a blood sample, from the subject.

In some embodiments, a composition comprising a therapeutically effective amount of at least one anti-Aβ protofibril antibody is administered once every week. In some embodiments, a composition comprising a therapeutically effective amount of at least one anti-Aβ protofibril antibody is administered once every two weeks. In some embodiments, a composition comprising a therapeutically effective amount of at least one anti-Aβ protofibril antibody is administered once every three weeks. In some embodiments, a composition comprising a therapeutically effective amount of at least one anti-Aβ protofibril antibody is administered once every four weeks. In some embodiments, a composition comprising a therapeutically effective amount of at least one anti-Aβ protofibril antibody is administered once every month.

In some embodiments, a composition comprising a therapeutically effective amount of BAN2401 is administered once every week. In some embodiments, a composition comprising a therapeutically effective amount of BAN2401 is administered once every two weeks. In some embodiments, a composition comprising a therapeutically effective amount of BAN2401 is administered once every three weeks. In some embodiments, a composition comprising a therapeutically effective amount of BAN2401 is administered once every four weeks. In some embodiments, a composition comprising a therapeutically effective amount of BAN2401 is administered once every month.

In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every week. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every two weeks. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every three weeks. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every four weeks. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every month.

In some embodiments, a composition comprising 2.5 mg/kg, 5 mg/kg, 7.5 mg/kg, or 10 mg/kg of BAN2401 relative to body weight of the subject is administered to the subject once every week. In some embodiments, a composition comprising 2.5 mg/kg, 5 mg/kg, 7.5 mg/kg, or 10 mg/kg of BAN2401 relative to body weight of the subject is administered to the subject once every two weeks. In some embodiments, a composition comprising 2.5 mg/kg, 5 mg/kg, 7.5 mg/kg, or 10 mg/kg of BAN2401 relative to body weight of the subject is administered to the subject once every three weeks. In some embodiments, a composition comprising 2.5 mg/kg, 5 mg/kg, 7.5 mg/kg, or 10 mg/kg of BAN2401 relative to body weight of the subject is administered to the subject once every four weeks. In some embodiments, a composition comprising 2.5 mg/kg, 5 mg/kg, 7.5 mg/kg, or 10 mg/kg of BAN2401 relative to body weight of the subject is administered to the subject once every month.

In some embodiments, a composition comprising 10 mg/kg of BAN2401 relative to body weight of the subject is administered to the subject once every two weeks. In some embodiments, a composition comprising 10 mg/kg of BAN2401 relative to body weight of the subject is administered to the subject once every month. In some embodiments, a reduced dosage frequency of BAN2401 and/or a reduced concentration of BAN2401 is administered if the initial dosing (e.g., every two or four weeks for 6-12 months or more) increases an Aβ42/40 ratio when comparing the ratio in a sample, e.g., a blood sample, before and after the initial treatment.

### Composition Comprising at Least One Anti-Aβ Protofibril Antibody

In some embodiments, the at least one anti-Aβ protofibril antibody is comprised in a composition. In some embodiments, the composition consists of at least one anti-Aβ protofibril antibody. In some embodiments, the antibody is present at a concentration of 50-250 mg/ML, e.g., 100-200 mg/mL. In some embodiments, the composition comprises at least one anti-Aβ protofibril antibody and further comprises at least one additional active and/or inactive component. In some embodiments, the at least one additional component can comprise one or more suitable physiologically acceptable excipients for human and/or veterinary use.

The compositions of the present disclosure may be in the form of a tablet, pill, capsule, solution, and/or any other suitable form deemed appropriate by one of ordinary skill in the art. The route of administration of the compositions of the present disclosure may be any suitable route, including intravenous, subcutaneous, oral, and nasal. In some embodiments, the composition is formulated as a sterile, non-pyrogenic liquid for intravenous administration. In some embodiments, the composition is a saline solution.

In some embodiments, the at least one additional component in the composition comprises buffer(s). In some embodiments, the at least one additional component comprises emulsifier(s). In some embodiments, the at least one additional component comprises sodium citrate, sodium chloride, histidine, arginine, arginine hydrochloride, and/or polysorbate 80. In some embodiments, the sodium citrate may be present at a concentration ranging from 1 mM to 150 mM. In some embodiments, the sodium citrate may be present at a concentration of 25 mM. In some embodiments, the sodium citrate may be present at a concentration of 50 mM. In some embodiments, the sodium chloride may be present at a concentration ranging from 25 mM to 250 mM. In some embodiments, the arginine may be present at a concentration ranging from 240 mM to 360 mM. In some embodiments, the arginine hydrochloride may be present at a concentration ranging from 100 mM to 250 mM. In some embodiments, the histidine may be present at a concentration ranging from 10 mM to 50 mM. In some embodiments, the sodium citrate may be present at a concentration of 125 mM. In some embodiments, the polysorbate 80 may be present at a concentration ranging from 0.001% (w/v) to 2% (w/v). In some embodiments, the polysorbate 80 may be present at a concentration of 0.02% (w/v). In some embodiments, the polysorbate 80 may be present at a concentration of 0.05% (w/v).

In some embodiments, the composition is a liquid dosage form comprising at least one anti-Aβ protofibril antibody, such as BAN2401, and further comprising, for instance, sodium citrate, sodium chloride, and polysorbate 80. In some embodiments, the composition is a liquid dosage form comprising 50 mmol/L citrate, 350 mmol/L arginine, and 0.05% polysorbate 80.

In some embodiments, the composition is a liquid dosage form comprising at least one anti-Aβ protofibril antibody, such as BAN2401, and further comprising, for instance, arginine hydrochloride, histidine, and polysorbate 80. In some embodiments, the composition is a liquid dosage form comprising 25 mmol/L histidine, 200 mmol/L arginine, 0.05% polysorbate 80. PCT/IB2021/000155 (WO2021/186245) is incorporated herein by reference for suitable intravenous and subcutaneous formulations.

Concomitant Administration of at Least One Anti-Aβ Protofibril Antibody and at Least One Alzheimer's Disease Medication Other than BAN2401

In some embodiments, provided herein is a method of treating a subject, e.g., one having Pre-AD or early Alzheimer's disease, comprising concomitantly administering a therapeutically effective amount of at least one anti-Aβ protofibril antibody such as BAN2401 and a therapeutically effective amount of at least one Alzheimer's disease medication other than BAN2401. In some embodiments, provided herein is a method of reducing and/or slowing clinical decline in a subject, e.g., one having Pre-AD or early Alzheimer's disease, comprising concomitantly administering a therapeutically effective amount of at least one anti-Aβ protofibril antibody such as BAN2401 and a therapeutically effective amount of at least one Alzheimer's disease medication other than BAN2401. The at least one additional therapy may comprise an additional anti-Aβ antibody such as aducanumab. In some embodiments, the at least one additional therapy may comprise a BACE inhibitor and/or an anti-tau antibody. In some embodiments, the additional therapy is given in place of an anti-Aβ protofibril antibody such as BAN2401 if initial treatment with the first antibody does not lead to an increase in the Aβ42/40 ratio, or the additional therapy is given in combination with an increased dosage or frequency of the first antibody. In some embodiments, the additional therapy is given in combination with a reduced dosage or administration frequency of an anti-Aβ protofibril antibody such as BAN2401 if initial treatment with the first antibody leads to an increase in the Aβ42/40 ratio.

In some embodiments, provided herein is a method of treating a subject having pre-AD, or a patient that is symptomatic for Alzheimer's disease (e.g., early Alzheimer's disease), comprising concomitantly administering a therapeutically effective amount of at least one anti-Aβ protofibril antibody such as BAN2401 and a therapeutically effective amount of an anti-tau antibody or antigen binding fragment thereof that is capable of binding to human tau, e.g., the anti-tau antibody or antigen binding fragment comprises E2814 or an antigen binding fragment thereof. E2814 is disclosed in US 2019/0112364 A1 as clone 7G6-HCzu25/LCzu18, the sequences of which are incorporated by reference herein. In some embodiments, provided herein is a method of reducing and/or slowing clinical decline in a subject, e.g., one having pre-AD, or a patient that is symptomatic for Alzheimer's disease (e.g., early Alzheimer's disease), comprising concomitantly administering a therapeutically effective amount of at least one anti-Aβ protofibril antibody such as BAN2401 and a therapeutically effective amount of an anti-tau antibody or antigen binding fragment thereof that is capable of binding to human tau, e.g., the anti-tau antibody or antigen binding fragment comprises E2814 or an antigen binding fragment thereof. E2814 is disclosed in US 2019/0112364 A1 as clone 7G6-HCzu25/LCzu18, the sequences of which are incorporated by reference herein. In some embodiments, the isolated anti-tau antibody or antigen binding fragment thereof that is capable of binding to human tau comprises six CDRs (HCDR1, HCDR2, HCDR3, LCDR1, LCDR2, and LCDR3) comprising the amino acid sequences of SEQ ID NO:15 (HCDR1), SEQ ID NO:16 (HCDR2), SEQ ID NO:17 (HCDR3), SEQ ID NO:18 (LCDR1), SEQ ID NO:19 (LCDR2), and SEQ ID NO:20 (LCDR3). See, e.g., Table 11. In some embodiments, the isolated anti-tau antibody or antigen binding fragment thereof that is capable of binding to human tau comprises six CDRs (HCDR1, HCDR2, HCDR3, LCDR1, LCDR2, and LCDR3) from a heavy chain variable region of SEQ ID NO: 21 and a light chain variable region of SEQ ID NO: 22. In some embodiments, the anti-tau antibody or antigen binding fragment thereof that is capable of binding to human tau comprises a heavy chain variable region of SEQ ID NO: 21 and a light chain variable region of SEQ ID NO: 22. See, e.g., Table 12. In some embodiments, the heavy chain constant region comprises SEQ ID NO: 23. In some embodiments, the heavy chain constant region comprises SEQ ID NO: 24. See, e.g., Table 13.

In some embodiments, a patient that is symptomatic for Alzheimer's disease is administered the anti-Aβ protofibril antibody (e.g., BAN2401) for at least 24 weeks then administered the isolated anti-tau antibody or antigen binding fragment thereof that is capable of binding to human tau (e.g., E2814) in conjunction with the isolated anti-Aβ protofibril antibody. In some embodiments, a patient that is symptomatic for Alzheimer's disease is administered the anti-Aβ protofibril antibody, e.g., for 24 weeks or until the patient's Aβ42/40 ratio increases above a threshold (e.g., 0.092), then administered the isolated anti-tau antibody or antigen binding fragment thereof that is capable of binding to human tau in conjunction with the isolated anti-Aβ protofibril antibody. In some embodiments, a patient that is symptomatic for Alzheimer's disease is administered the anti-Aβ protofibril antibody for 24 weeks or until the patient is amyloid negative, then administered the isolated anti-tau antibody or antigen binding fragment thereof that is capable of binding to human tau in conjunction with the isolated anti-Aβ protofibril antibody.

In some embodiments, the patient is asymptomatic for Alzheimer's disease (pre-AD) and is first administered an isolated anti-tau antibody or antigen binding fragment thereof that is capable of binding to human tau (e.g., E2814), e.g., for 52 weeks before being administered the isolated anti-tau antibody or antigen binding fragment thereof that is capable of binding to human tau in conjunction with an isolated anti-Aβ protofibril antibody (e.g., BAN2401). In some embodiments, a patient that is asymptomatic for Alzheimer's disease is administered the isolated anti-tau antibody or antigen binding fragment thereof that is capable of binding to human tau for 52 weeks or until the patient's Aβ42/40 ratio increases above a threshold (e.g., 0.092), then administered the isolated anti-tau antibody or antigen binding fragment thereof that is capable of binding to human tau in conjunction with the isolated anti-Aβ protofibril antibody. In some embodiments, a patient that is asymptomatic for Alzheimer's disease is administered the isolated anti-tau antibody or antigen binding fragment thereof that is capable of binding to human tau for 52 weeks or until the patient is amyloid negative, then administered the isolated anti-tau antibody or antigen binding fragment thereof that is capable of binding to human tau in conjunction with the isolated anti-Aβ protofibril antibody.

In some embodiments, the at least one Alzheimer's disease medication is chosen from elenbecestat, donepezil, galantamine, memantine, and rivastigmine. In some embodiments, the at least one Alzheimer's disease medication is a combination of donepezil and memantine. In some embodiments, the at least one additional therapeutic agent comprises one or more of BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof. In some embodiments, the at least one additional therapeutic agent is a BACE inhibitor. In some embodiments, the BACE inhibitor is chosen from CNP520, BI-1181181, LY2886721, LY3202626, PF-06751979, RG7129, atabecestat, elenbecestat, lanabecestat, and verubecestat. In some embodiments, the BACE inhibitor is elenbecestat. In some embodiments, the BACE inhibitor is chosen from CNP520, BI-1181181, LY2886721, LY3202626, PF-06751979, RG7129, atabecestat, elenbecestat, lanabecestat, and verubecestat.

In some embodiments, donepezil may be administered at its approved dose. In some embodiments, galantamine may be administered at its approved dose. In some embodiments, memantine may be administered at its approved dose. In some embodiments, rivastigmine may be administered at its approved dose.

In some embodiments, elenbecestat may be administered at a dose ranging from 5 mg/day to 100 mg/day, 10 mg/day to 75 mg/day, 5 mg/day to 50 mg/day, or 15 mg/day to 50 mg/day. In some embodiments, elenbecestat may be administered at a dose ranging from about 5 mg/day to about 100 mg/day, about 10 mg/day to about 75 mg/day, about 5 mg/day to about 50 mg/day, or about 15 mg/day to about 50 mg/day. In some embodiments, elenbecestat may be administered at a dose of 5 mg/day, 10 mg/day, 15 mg/day, 20 mg/day, 25 mg/day, 30 mg/day, or 50 mg/day dosage. In some embodiments, elenbecestat may be administered at a dose of 5 mg/day. In some embodiments, elenbecestat may be administered at a dose of 15 mg/day. In some embodiments, elenbecestat may be administered at a dose of 50 mg/day.

In some embodiments, elenbecestat may be administered at a dose ranging from 5 mg/day to 100 mg/day, 10 mg/day to 75 mg/day, 5 mg/day to 50 mg/day, or 15 mg/day to 50 mg/day. In some embodiments, elenbecestat may be administered at a dose ranging from about 5 mg/day to about 100 mg/day, about 10 mg/day to about 75 mg/day, about 5 mg/day to about 50 mg/day, or about 15 mg/day to about 50 mg/day. In some embodiments, elenbecestat may be administered at a dose of 5 mg/day, 10 mg/day, 15 mg/day, 20 mg/day, 25 mg/day, 30 mg/day, or 50 mg/day dosage. In some embodiments, elenbecestat may be administered at a dose of 5 mg/day. In some embodiments, elenbecestat may be administered at a dose of 15 mg/day. In some embodiments, elenbecestat may be administered at a dose of 50 mg/day.

### Therapeutic Effect

In various embodiments, provided herein is a method of reducing clinical decline in a subject having early Alzheimer's disease comprising administering to said subject a composition comprising a therapeutically effective amount of at least one anti-Aβ protofibril antibody disclosed herein. In some embodiments, the subject having early Alzheimer's disease has been diagnosed as having mild cognitive impairment due to Alzheimer's disease-intermediate likelihood and/or has been diagnosed as having mild Alzheimer's disease dementia. In some embodiments, the subject having early Alzheimer's disease is ApoE4-positive.

Any of the anti-Aβ protofibril antibodies, therapeutically acceptable amounts thereof, dosing regimens therefor, and compositions comprising the same that are disclosed herein may be used in the method of reducing clinical decline in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, once every month, once every five weeks, once every six weeks, once every seven weeks, once every eight weeks, once every two months, once every nine weeks, once every ten weeks, once every eleven weeks, once every twelve weeks, once every three months (quarterly), once every fourteen weeks, once every sixteen weeks, once every four months, once every eighteen weeks, once every twenty weeks, once every five months, once every 22 weeks, once every 24 weeks, once every six months (semi-annually), once every seven months, once every eight months, once every nine months, once every ten months, once every eleven months, once every twelve months (annually), once every thirteen months, once every fourteen months, once every fifteen months, once every sixteen months, once every seventeen months, or once every eighteen months. In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, or at least 46% relative to placebo as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 35% relative to placebo as determined by ADCOMS. In some embodiments, the clinical decline is reduced by 20% to 30% relative to placebo as determined by ADCOMS. In some embodiments, the clinical decline is reduced by 27% to 35% relative to placebo as determined by ADCOMS. In some embodiments, the clinical decline is reduced by at least 20% relative to placebo as determined by ADCOMS. In some embodiments, the clinical decline is reduced by at least 35% relative to placebo as determined by ADCOMS. In some embodiments, the clinical decline is reduced by at least 20% as determined by ADCOMS. In some embodiments, the clinical decline is reduced by at least 30% as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 45% relative to placebo as determined by ADCOMS after 6 months of administration of the composition comprising a therapeutically effective amount of at least one anti-Aβ protofibril antibody. In some embodiments, the clinical decline is reduced by at least 35% relative to placebo as determined by ADCOMS after 12 months of administration of the composition comprising a therapeutically effective amount of at least one anti-Aβ protofibril antibody. In some embodiments, the clinical decline is reduced by at least 30% relative to placebo as determined by ADCOMS after 18 months of administration of the composition comprising a therapeutically effective amount of at least one anti-Aβ protofibril antibody. In some embodiments, the clinical decline is reduced by at least 46% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, or at least 52% relative to placebo as determined by ADCOMS, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 33% relative to placebo as determined by ADCOMS, wherein the subject has been diagnosed as having mild cognitive impairment due to Alzheimer's disease-intermediate likelihood. In some embodiments, the clinical decline is reduced by at least 20%, such as by at 25% or at least 28%, relative to placebo as determined by ADCOMS, wherein the subject has been diagnosed as having mild cognitive impairment due to Alzheimer's disease-intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease-intermediate likelihood. In some embodiments, the clinical decline is reduced by at least 25%, such as by at least 30%, at least 35%, at least 40%, at least 45%, at least 50%, or at least 52% relative to placebo as determined by ADCOMS, wherein the subject has been diagnosed as having mild cognitive impairment due to Alzheimer's disease-intermediate likelihood. In some embodiments, the clinical decline is reduced by at least 52% relative to placebo as determined by ADCOMS, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-intermediate likelihood is reduced by at least 30% relative to placebo as determined by ADCOMS after 6 months of administration of the composition comprising a therapeutically effective amount of at least one anti-Aβ protofibril antibody. In some embodiments, the clinical decline in the subject diagnosed as having mild cognitive impairment due to Alzheimer's disease-intermediate likelihood is reduced by at least 25% relative to placebo as determined by ADCOMS after 12 months of administration of the composition comprising a therapeutically effective amount of at least one anti-Aβ protofibril antibody. In some embodiments, the clinical decline in the subject diagnosed as having mild cognitive impairment due to Alzheimer's disease-intermediate likelihood is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild cognitive impairment due to Alzheimer's disease-intermediate likelihood is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, or at least 33% relative to placebo as determined by ADCOMS, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild cognitive impairment due to Alzheimer's disease-intermediate likelihood. In some embodiments, the clinical decline is reduced by at least 20%, such as by at 25%, at least 28%, or at least 33%, relative to placebo as determined by ADCOMS, wherein the subject has been diagnosed as having mild cognitive impairment due to Alzheimer's disease-intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease-intermediate likelihood. In some embodiments, the clinical decline is reduced by at least 33% relative to placebo as determined by ADCOMS, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-intermediate likelihood is reduced by at least 33% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, or at least 78% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 80% relative to placebo as determined by ADCOMS, wherein the subject has been diagnosed as having Alzheimer's disease dementia. In some embodiments, the clinical decline is reduced by 35% to 78% relative to placebo as determined by ADCOMS, wherein the subject has been diagnosed as having Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the clinical decline is reduced by at least 50%, such as by at least 52% or at least 53% relative to placebo as determined by ADCOMS, wherein the subject has been diagnosed as having mild Alzheimer's disease dementia. In some embodiments, the clinical decline is reduced by at least 70%, such as by at least 75% or at least 78%, relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 70% relative to placebo as determined by ADCOMS after 6 months of administration of the composition comprising a therapeutically effective amount of at least one anti-Aβ protofibril antibody. In some embodiments, the clinical decline in the subject diagnosed as having mild Alzheimer's disease dementia is reduced by at least 50% relative to placebo as determined by ADCOMS after 12 months of administration of the composition comprising a therapeutically effective amount of at least one anti-Aβ protofibril antibody. In some embodiments, the clinical decline in the subject diagnosed as having mild Alzheimer's disease dementia is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild Alzheimer's disease dementia is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild Alzheimer's disease dementia. In some embodiments, the clinical decline is reduced by at least 20%, such as by at 25%, at least 28%, or at least 35%, relative to placebo as determined by ADCOMS, wherein the subject has been diagnosed as having mild Alzheimer's disease dementia. In some embodiments, the clinical decline is reduced by at least 25%, such as by at least 30% or at least 35%, relative to placebo as determined by ADCOMS, wherein the subject has been diagnosed as having mild Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 35% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 75%, at least 80%, at least 85%, at least 90%, at least 95%, at least 100%, at least 110%, at least 120%, at least 130%, at least 140%, or at least 150% relative to placebo as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 150% relative to placebo as determined by ADAS-cog. In some embodiments, the clinical decline is reduced by 45% to 145% relative to placebo as determined by ADAS-cog. In some embodiments, the clinical decline is reduced by 45% to 55% relative to placebo as determined by ADAS-cog. In some embodiments, the clinical decline is reduced by at least 30% relative to placebo as determined by ADAS-cog. In some embodiments, the clinical decline is reduced by at least 35% relative to placebo as determined by ADAS-cog. In some embodiments, the clinical decline is reduced by at least 40% as determined by ADAS-cog. In some embodiments, the clinical decline is reduced by at least 45% as determined by ADAS-cog. In some embodiments, the clinical decline is reduced by at least 47% as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 100%, such as at least 120% or at least 140%, relative to placebo as determined by ADAS-cog after 6 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 12 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 18 months of administration of the composition comprising a therapeutically effective amount of at least one anti-Aβ protofibril antibody. In some embodiments, the clinical decline is reduced by at least 47%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 56%, at least 47%, at least 48%, at least 49%, at least 50%, at least 51%, at least 52%, at least 53%, at least 54%, at least 55%, at least 56%, at least 57%, or at least 58% relative to placebo as determined by ADAS-cog, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject has been diagnosed as having mild cognitive impairment due to Alzheimer's disease-intermediate likelihood. In some embodiments, the clinical decline is reduced by at least 50%, such as by at 52%, at least 55%, or at least 58%, relative to placebo as determined by ADAS-cog, wherein the subject has been diagnosed as having mild cognitive impairment due to Alzheimer's disease-intermediate likelihood. In some embodiments, the clinical decline is reduced by at least 58% relative to placebo as determined by ADAS-cog, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-intermediate likelihood is reduced by at least 58% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, or at least 41% relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 50% relative to placebo as determined by ADAS-cog, wherein the subject has been diagnosed as having mild Alzheimer's disease dementia. In some embodiments, the clinical decline is reduced by at least 35%, such as by at 38%, at least 40%, or at least 41%, relative to placebo as determined by ADAS-cog, wherein the subject has been diagnosed as having mild Alzheimer's disease dementia. In some embodiments, the clinical decline is reduced by at least 41% relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 41% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% relative to placebo as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 60% relative to placebo as determined by CDR-SB. In some embodiments, the clinical decline is reduced by 25% to 60% relative to placebo as determined by CDR-SB. In some embodiments, the clinical decline is reduced by 25% to 50% relative to placebo as determined by CDR-SB. In some embodiments, the clinical decline is reduced by at least 20% relative to placebo as determined by CDR-SB. In some embodiments, the clinical decline is reduced by at least 30% relative to placebo as determined by CDR-SB. In some embodiments, the clinical decline is reduced by at least 25%, such as at least 26% or at least 28%, as determined by CDR-SB. In some embodiments, the clinical decline is reduced by at least 30%, such as at least 35% or at least 38%, as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 30%, such as at least 35% or at least 40%, relative to placebo as determined by CDR-SB after 6 months of administration of the composition comprising a therapeutically effective amount of at least one anti-Aβ protofibril antibody. In some embodiments, the clinical decline is reduced by at least 30%, such as at least 35% or at least 45%, relative to placebo as determined by CDR-SB after 12 months of administration of the composition comprising a therapeutically effective amount of at least one anti-Aβ protofibril antibody. In some embodiments, the clinical decline is reduced by at least 20%, such as at least 25%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, or at least 14% relative to placebo as determined by CDR-SB, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 10% to 20% relative to placebo as determined by CDR-SB, wherein the subject has been diagnosed as having mild cognitive impairment due to Alzheimer's disease-intermediate likelihood. In some embodiments, the clinical decline is reduced by at least 5%, such as by at 10%, at least 12%, or at least 14%, relative to placebo as determined by CDR-SB, wherein the subject has been diagnosed as having mild cognitive impairment due to Alzheimer's disease-intermediate likelihood. In some embodiments, the clinical decline is reduced by at least 14% relative to placebo as determined by CDR-SB, wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-intermediate likelihood is reduced by at least 14% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, or at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 60% relative to placebo as determined by CDR-SB, wherein the subject has been diagnosed as having mild Alzheimer's disease dementia. In some embodiments, the clinical decline is reduced by at least 45%, such as by at 48%, at least 50%, or at least 51%, relative to placebo as determined by CDR-SB, wherein the subject has been diagnosed as having mild Alzheimer's disease dementia. In some embodiments, the clinical decline is reduced by at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 51% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of at least one anti-Aβ protofibril antibody. In some embodiments, the reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the reduction in clinical decline is determined after 1 month of administration of the composition comprising a therapeutically effective amount of at least one anti-Aβ protofibril antibody. In some embodiments, the reduction in clinical decline is determined after 6 months of administration of the composition comprising a therapeutically effective amount of at least one anti-Aβ protofibril antibody. In some embodiments, the reduction in clinical decline is determined after 12 months of administration of the composition comprising a therapeutically effective amount of at least one anti-Aβ protofibril antibody. In some embodiments, the reduction in clinical decline is determined after 18 months of administration of the composition comprising a therapeutically effective amount of at least one anti-Aβ protofibril antibody. In some embodiments, the reduction in clinical decline is determined after 60 months of administration of the composition comprising a therapeutically effective amount of at least one anti-Aβ protofibril antibody. In some embodiments, the reduction in clinical decline is determined after 63 months of administration of the composition comprising a therapeutically effective amount of at least one anti-Aβ protofibril antibody.

In some embodiments, the reduction in clinical decline is determined after administration of a composition comprising a therapeutically effective amount of BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of BAN2401. In some embodiments, the reduction in clinical decline is determined after 1 month, 6 months, 12 months, 18 months, 60 months, and/or 63 months of administration of the composition comprising a therapeutically effective amount of BAN2401. In some embodiments, the reduction in clinical decline is determined after 1 month of administration of the composition comprising a therapeutically effective amount of BAN2401. In some embodiments, the reduction in clinical decline is determined after 6 months of administration of the composition comprising a therapeutically effective amount of BAN2401. In some embodiments, the reduction in clinical decline is determined after 12 months of administration of the composition comprising a therapeutically effective amount of BAN2401. In some embodiments, the reduction in clinical decline is determined after 18 months of administration of the composition comprising a therapeutically effective amount of BAN2401. In some embodiments, the reduction in clinical decline is determined after 60 months of administration of the composition comprising a therapeutically effective amount of BAN2401. In some embodiments, the reduction in clinical decline is determined after 63 months of administration of the composition comprising a therapeutically effective amount of BAN2401.

In some embodiments, the subject is ApoE4-positive.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, or at least 74% relative to placebo as determined by ADCOMS, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ antibody.

In some embodiments, the clinical decline is reduced by 60% to 80%, such as by 63% to 74%, relative to placebo as determined by ADCOMS, wherein the subject is ApoE4-positive. In some embodiments, the clinical decline is reduced by at least 60%, such as at least 63%, relative to placebo as determined by ADCOMS, wherein the subject is ApoE4-positive. In some embodiments, the clinical decline is reduced by at least 65%, such as at least 67%, relative to placebo as determined by ADCOMS, wherein the subject is ApoE4-positive. In some embodiments, the clinical decline is reduced by at least 70%, such as at least 74%, relative to placebo as determined by ADCOMS, wherein the subject is ApoE4-positive.

In some embodiments, the clinical decline in the ApoE4-positive subject is reduced by at least 70% relative to placebo as determined by ADCOMS after 6 months of administration of the composition comprising a therapeutically effective amount of at least one anti-Aβ protofibril antibody. In some embodiments, the clinical decline in the ApoE4-positive subject is reduced by at least 60% relative to placebo as determined by ADCOMS after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by ADCOMS after 18 months of administration of the composition comprising a therapeutically effective amount of at least one anti-Aβ protofibril antibody. In some embodiments, the clinical decline in the ApoE4-positive subject is reduced by at least 63%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 290%, at least 295%, at least 300%, at least 305%, at least 310%, at least 315%, at least 320%, at least 325%, at least 330%, or at least 331% relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced 70% to 400%, such as 80% to 350%, relative to placebo as determined by ADAS-cog, wherein the subject is ApoE4-positive. In some embodiments, the clinical decline is reduced by at least 70%, such as at least 75% or at least 80%, relative to placebo as determined by ADAS-cog, wherein the subject is ApoE4-positive. In some embodiments, the clinical decline is reduced by at least 80%, such as at least 90% or at least 100%, relative to placebo as determined by ADAS-cog, wherein the subject is ApoE4-positive. In some embodiments, the clinical decline is reduced by at least 300%, such as at least 330%, relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 300% relative to placebo as determined by ADAS-cog after 6 months of administration of the composition comprising a therapeutically effective amount of at least one anti-Aβ protofibril antibody. In some embodiments, the clinical decline in the ApoE4-positive subject is reduced by at least 80%, such as at least 90% or at least 100%, relative to placebo as determined by ADAS-cog after 12 months of administration of the composition comprising a therapeutically effective amount of at least one anti-Aβ protofibril antibody. In some embodiments, the clinical decline in the ApoE4-positive subject is reduced by at least 70%, such as at least 75%, at least 80%, or at least 84%, relative to placebo as determined by ADAS-cog after 18 months of administration of the composition comprising a therapeutically effective amount of at least one anti-Aβ protofibril antibody. In some embodiments, the clinical decline in the ApoE4-positive subject is reduced by at least 84%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, or at least 87% relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 35% to 150%, such as 40% to 100% or 45% to 90%, relative to placebo as determined by CDR-SB, wherein the subject is ApoE4-positive. In some embodiments, the clinical decline is reduced by at least 35%, such as at least 40% or at least 45%, relative to placebo as determined by CDR-SB, wherein the subject is ApoE4-positive. In some embodiments, the clinical decline is reduced by at least 50%, such as at least 55% or at least 60%, relative to placebo as determined by CDR-SB, wherein the subject is ApoE4-positive. In some embodiments, the clinical decline is reduced by at least 70%, such as at least 80% or at least 85%, relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 35%, such as at least 40% or at least 45%, relative to placebo as determined by CDR-SB after 6 months of administration of the composition comprising a therapeutically effective amount of at least one anti-Aβ protofibril antibody. In some embodiments, the clinical decline in the ApoE4-positive subject is reduced by at least 70%, such as at least 75% or at least 80%, relative to placebo as determined by CDR-SB after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by CDR-SB after 18 months of administration of the composition comprising a therapeutically effective amount of at least one anti-Aβ protofibril antibody. In some embodiments, the clinical decline in the ApoE4-positive subject is reduced by at least 60%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, or at least 59% relative to placebo as determined by ADCOMS, wherein the subject is ApoE4-positive, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 70%, such as 38% to 59%, relative to placebo as determined by ADCOMS, wherein the subject is ApoE4-positive, and wherein the subject has been diagnosed as having mild cognitive impairment due to Alzheimer's disease-intermediate likelihood. In some embodiments, the clinical decline is reduced by at least 30%, such as at least 35% or at least 38%, relative to placebo as determined by ADCOMS, wherein the subject is ApoE4-positive, and wherein the subject has been diagnosed as having mild cognitive impairment due to Alzheimer's disease-intermediate likelihood. In some embodiments, the clinical decline is reduced by at least 45%, such as at least 50% or at least 53%, relative to placebo as determined by ADCOMS, wherein the subject is ApoE4-positive, and wherein the subject has been diagnosed as having mild cognitive impairment due to Alzheimer's disease-intermediate likelihood. In some embodiments, the clinical decline is reduced by at least 50%, such as at least 55% or at least 59%, relative to placebo as determined by ADCOMS, wherein the subject is ApoE4-positive, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease-intermediate likelihood is reduced by at least 50%, such as at least 55%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease-intermediate likelihood is reduced by at least 30%, such as at least 35%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease-intermediate likelihood is reduced by at least 45%, such as at least 50% or at least 55%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 100%, such as at least 110%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 100%, such as at least 110%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70% or at least 75%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADAS-Cog, wherein the subject is ApoE4-positive, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 300%, such as 45% to 250% or 50% to 250%, relative to placebo as determined by ADAS-Cog, wherein the subject is ApoE4-positive, and wherein the subject has been diagnosed as having mild cognitive impairment due to Alzheimer's disease-intermediate likelihood. In some embodiments, the clinical decline is reduced by at least 40%, such as at least 45% or at least 50%, relative to placebo as determined by ADAS-Cog, wherein the subject is ApoE4-positive, and wherein the subject has been diagnosed as having mild cognitive impairment due to Alzheimer's disease-intermediate likelihood. In some embodiments, the clinical decline is reduced by at least 60, such as at least 70%, at least 75%, or at least 80%, relative to placebo as determined by ADAS-Cog, wherein the subject is ApoE4-positive, and wherein the subject has been diagnosed as having mild cognitive impairment due to Alzheimer's disease-intermediate likelihood. In some embodiments, the clinical decline is reduced by at least 100%, such as at least 150% or at least 200% relative to placebo as determined by ADAS-Cog, wherein the subject is ApoE4-positive, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease-intermediate likelihood is reduced by at least 100%, such as at least 150% or at least 200%, relative to placebo as determined by ADAS-cog after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease-intermediate likelihood is reduced by at least 40%, such as at least 45% or at least 50%, relative to placebo as determined by ADAS-cog after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease-intermediate likelihood is reduced by at least 50%, such as at least 60%, at least 70%, or at least 75%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is ApoE4-positive, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 90%, such as 25% to 80% or 30% to 75%, relative to placebo as determined by CDR-SB, wherein the subject is ApoE4-positive, and wherein the subject has been diagnosed as having mild cognitive impairment due to Alzheimer's disease-intermediate likelihood. In some embodiments, the clinical decline is reduced by at least 25%, such as at least 30%, relative to placebo as determined by CDR-SB, wherein the subject is ApoE4-positive, and wherein the subject has been diagnosed as having mild cognitive impairment due to Alzheimer's disease-intermediate likelihood. In some embodiments, the clinical decline is reduced by at least 30%, such as at least 35% or 40%, relative to placebo as determined by CDR-SB, wherein the subject is ApoE4-positive, and wherein the subject has been diagnosed as having mild cognitive impairment due to Alzheimer's disease-intermediate likelihood. In some embodiments, the clinical decline is reduced by at least 35%, such as at least 40% or 45%, relative to placebo as determined by CDR-SB, wherein the subject is ApoE4-positive, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease-intermediate likelihood is reduced by at least 35%, such as at least 40% or at least 45%, relative to placebo as determined by CDR-SB composition comprising a therapeutically effective amount of at least one anti-Aβ protofibril antibody. In some embodiments, the clinical decline in the ApoE4-positive subject diagnosed as mild cognitive impairment due to Alzheimer's disease-intermediate likelihood is reduced by at least 20%, such as at least 25% or at least 30%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease-intermediate likelihood is reduced by at least 35%, such as at least 40%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, or at least 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 76% to 119% relative to placebo as determined by ADCOMS, wherein the subject is ApoE4-positive, and wherein the subject has been diagnosed as having mild Alzheimer's disease dementia. In some embodiments, the clinical decline is reduced by 76% relative to placebo as determined by ADCOMS, wherein the subject is ApoE4-positive, and wherein the subject has been diagnosed as having mild Alzheimer's disease dementia. In some embodiments, the clinical decline is reduced by 113% relative to placebo as determined by ADCOMS, wherein the subject is ApoE4-positive, and wherein the subject has been diagnosed as having mild Alzheimer's disease dementia. In some embodiments, the clinical decline is reduced by 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, at least 174%, at least 175%, at least 176%, at least 177%, at least 178%, at least 179%, at least 180%, at least 190%, at least 200%, at least 210%, at least 220%, at least 230%, at least 240%, at least 250%, at least 275%, at least 300%, at least 325%, at least 350%, at least 375%, at least 400%, at least 425%, at least 450%, at least 475%, at least 500%, at least 550%, at least 600%, at least 650%, at least 700%, at least 750%, at least 800%, at least 850%, at least 900%, at least 950%, at least 1000%, at least 1001%, at least 1002%, at least 1003%, at least 1004%, at least 1005%, at least 1006%, at least 1007%, at least 1008%, at least 1009%, at least 1010%, at least 1011%, at least 1012%, at least 1013%, at least 1014%, at least 1015%, at least 1016%, at least 1017%, at least 1018%, at least 1019%, at least 1020%, at least 1021%, at least 1022%, or at least 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 58% to 1023% relative to placebo as determined by ADAS-Cog, wherein the subject is ApoE4-positive, and wherein the subject has been diagnosed as having mild Alzheimer's disease dementia. In some embodiments, the clinical decline is reduced by 58% relative to placebo as determined by ADAS-Cog, wherein the subject is ApoE4-positive, and wherein the subject has been diagnosed as having mild Alzheimer's disease dementia. In some embodiments, the clinical decline is reduced by 171% relative to placebo as determined by ADAS-Cog, wherein the subject is ApoE4-positive, and wherein the subject has been diagnosed as having mild Alzheimer's disease dementia. In some embodiments, the clinical decline is reduced by 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, or at least 174% relative to placebo as determined by CDR-SB, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 70% to 200%, such as 75% to 180% or 82% to 174%, relative to placebo as determined by CDR-SB, wherein the subject is ApoE4-positive, and wherein the subject has been diagnosed as having mild Alzheimer's disease dementia. In some embodiments, the clinical decline is reduced by at least 70%, such as at least 80% relative to placebo as determined by CDR-SB, wherein the subject is ApoE4-positive, and wherein the subject has been diagnosed as having mild Alzheimer's disease dementia. In some embodiments, the clinical decline is reduced by at least 75%, such as at least 80% or at least 85%, relative to placebo as determined by CDR-SB, wherein the subject is ApoE4-positive, and wherein the subject has been diagnosed as having mild Alzheimer's disease dementia. In some embodiments, the clinical decline is reduced by at least 150%, such as at least 160% or 170%, relative to placebo as determined by CDR-SB, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 70%, such as at least 75%, at least 80%, or at least 85%, relative to placebo as determined by CDR-SB composition comprising a therapeutically effective amount of at least one anti-Aβ protofibril antibody. In some embodiments, the clinical decline in the ApoE4-positive subject diagnosed as having mild Alzheimer's disease dementia is reduced by at least 130%, such as at least 140%, at least 150%, at least 160%, or at least 170%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70%, at least 75%, or at least 80%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the subject is ApoE4-negative.

In some embodiments, the clinical decline is reduced by at least 1%, at least 2%, at least 3%, at least 4%, at least 5%, at least 6%, at least 7%, at least 8%, at least 9%, at least 10%, at least 11%, or at least 12% relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 5% to 15% relative to placebo as determined by ADCOMS, wherein the subject is ApoE4-negative. In some embodiments, the clinical decline is reduced by at least 5%, such as at least 7%, relative to placebo as determined by ADCOMS, wherein the subject is ApoE4-negative. In some embodiments, the clinical decline is reduced by at least 10%, such as at least 12%, relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least −2% relative to placebo as determined by ADCOMS after 6 months of administration of the composition comprising a therapeutically effective amount of at least one anti-Aβ protofibril antibody. In some embodiments, the clinical decline in the ApoE4-negative subject is reduced by at least 10% relative to placebo as determined by ADCOMS after 12 months of administration of the composition comprising a therapeutically effective amount of at least one anti-Aβ protofibril antibody. In some embodiments, the clinical decline in the ApoE4-negative subject is reduced by at least 5%, such as at least 7%, relative to placebo as determined by ADCOMS after 18 months of administration of the composition comprising a therapeutically effective amount of at least one anti-Aβ protofibril antibody. In some embodiments, the clinical decline in the ApoE4-negative subject is reduced by at least 7%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, or at least 72% relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 80% relative to placebo as determined by ADAS-cog, wherein the subject is ApoE4-negative. In some embodiments, the clinical decline is reduced by at least 35%, such as at least 40% or at least 43%, relative to placebo as determined by ADAS-cog, wherein the subject is ApoE4-negative. In some embodiments, the clinical decline is reduced by at least 40%, such as at least 45% or at least 46%, relative to placebo as determined by ADAS-cog, wherein the subject is ApoE4-negative. In some embodiments, the clinical decline is reduced by at least 65%, such as at least 70% or at least 72%, relative to placebo as determined by ADAS-cog, wherein the subject is ApoE4-negative. In some embodiments, the clinical decline is reduced by at least 43%, relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is increased by 7%, 6%, 5%, 5%, 3%, 2%, or 1% relative to placebo as determined by CDR-SB, wherein the subject is ApoE4-negative. In some embodiments, the clinical decline is reduced by at least 1%, at least 2%, or at least 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody. In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, or at least 26% relative to placebo as determined by ADCOMS, wherein the subject is ApoE4-negative, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 15% to 26% relative to placebo as determined by ADCOMS, wherein the subject is ApoE4-negative, and wherein the subject has been diagnosed as having mild cognitive impairment due to Alzheimer's disease-intermediate likelihood. In some embodiments, the clinical decline is reduced by 15% relative to placebo as determined by ADCOMS, wherein the subject is ApoE4-negative, and wherein the subject has been diagnosed as having mild cognitive impairment due to Alzheimer's disease-intermediate likelihood. In some embodiments, the clinical decline is reduced by 26% relative to placebo as determined by ADCOMS, wherein the subject is ApoE4-negative, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, or at least 166% relative to placebo as determined by ADAS-Cog, wherein the subject is ApoE4-negative, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 200%, such as 60% to 180% or 65% to 170%, relative to placebo as determined by ADAS-Cog, wherein the subject is ApoE4-negative, and wherein the subject has been diagnosed as having mild cognitive impairment due to Alzheimer's disease-intermediate likelihood. In some embodiments, the clinical decline is reduced by at least 50%, such as at least 55% or at least 65%, relative to placebo as determined by ADAS-Cog, wherein the subject is ApoE4-negative, and wherein the subject has been diagnosed as having mild cognitive impairment due to Alzheimer's disease-intermediate likelihood. In some embodiments, the clinical decline is reduced by at least 70%, such as at least 75% or at least 80%, relative to placebo as determined by ADAS-Cog, wherein the subject is ApoE4-negative, and wherein the subject has been diagnosed as having mild cognitive impairment due to Alzheimer's disease-intermediate likelihood. In some embodiments, the clinical decline is reduced by at least 150%, such as at least 160%, relative to placebo as determined by ADAS-Cog, wherein the subject is ApoE4-negative, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least 150%, such as at least 160%, relative to placebo as determined by ADAS-Cog after 6 months of administration of the composition comprising a therapeutically effective amount of at least one anti-Aβ protofibril antibody. In some embodiments, the clinical decline in the ApoE4-negative subject is reduced by at least 70%, such as at least 75% or at least 80%, relative to placebo as determined by ADAS-Cog after 12 months of administration of the composition comprising a therapeutically effective amount of at least one anti-Aβ protofibril antibody. In some embodiments, the clinical decline in the ApoE4-negative subject is reduced by at least 50%, such as at least 60% or at least 65%, relative to placebo as determined by ADAS-Cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, or at least 5% relative to placebo as determined by CDR-SB, wherein the subject is ApoE4-negative, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 5% relative to placebo as determined by CDR-SB, wherein the subject is ApoE4-negative, and wherein the subject has been diagnosed as having mild cognitive impairment due to Alzheimer's disease-intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12% relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 10%, such as at least 12%, relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

Conversion of a Subject from Amyloid Positive to Amyloid Negative

In various embodiments, a subject is administered a composition comprising at least one anti-Aβ protofibril antibody disclosed herein before exhibiting cognitive symptoms of AD (pre-AD). In some embodiments, the pre-AD patient is amyloid negative, e.g., as determined by PET SUVr and/or by plasma biomarkers such as an Aβ42/40 ratio in a blood sample. In some embodiments, the pre-AD patient has an Aβ42/40 ratio of above 0.092, e.g., a ratio of about 0.092-0.094, and optionally the patient has intermediate amyloid β, e.g., as measured by PET SUVr prior to treatment. In some embodiments, a patient with pre-AD may be administered a treatment regimen comprising an anti-Aβ protofibril antibody (i.e. lecanemab) to prevent amyloid positivity. In some embodiments, a patient with pre-AD may be administered a treatment regimen comprising an anti-Aβ protofibril antibody (i.e. lecanemab) to delay onset of amyloid positivity. In some embodiments, a patient with pre-AD may be administered a treatment regimen comprising an anti-Aβ protofibril antibody (i.e. lecanemab) to prevent a worsening in one or more biomarkers of AD or brain measurement (e.g., by PET or MRI), e.g., of brain atrophy and/or amyloid accumulation. In some embodiments, a pre-AD patient who is amyloid negative is administered a reduced dose or dosing frequency as compared to a dose or frequency given to a patient who is amyloid positive (e.g., an intravenous infusion is given at less than 10 mg/kg or less than biweekly, or a subcutaneous administration is provided at less than 720 mg or less than weekly). In some embodiments, a pre-AD patient who is amyloid negative is moved to a maintenance dosing regimen sooner (e.g., in less than 18 months).

In some embodiments, a pre-AD patient has an Aβ42/40 ratio of below about 0.092, and the patient is administered a treatment regimen comprising an anti-Aβ protofibril antibody (i.e. lecanemab) to increase the Aβ42/40 ratio to at or above about 0.092. In some embodiments, the patient has intermediate amyloid β, e.g., as measured by PET SUVr prior to treatment. In some embodiments, treatment reduces the amyloid β, e.g., as measured by PET SUVr. In some embodiments, treatment converts the patient from amyloid positive to amyloid negative status, e.g., as assessed by the Aβ42/40 ratio and/or PET SUVr).

In various embodiments, also provided herein is a method of converting an amyloid-positive subject to an amyloid-negative subject. In some embodiments, said method comprises administering to said subject a composition comprising at least one anti-Aβ protofibril antibody disclosed herein. In some embodiments, said subject having early Alzheimer's disease has been diagnosed as having mild cognitive impairment due to Alzheimer's disease-intermediate likelihood and/or has been diagnosed as having mild Alzheimer's disease dementia. In some embodiments, the method further comprises evaluating the efficacy of a treatment by measuring an Aβ42/40 ratio before administering a first dose of the composition comprising the anti-Aβ protofibril antibody, and measuring again after administering the antibody, e.g., after 6-12 or 18 or 24 or 36 months of treatment. In some embodiments, the Aβ42/40 ratio is calculated by measuring a concentration of amyloid β 1-42 (Aβ42) and a concentration of amyloid β 1-40 (Aβ40) in a blood sample obtained from the subject to determine a first ratio of Aβ 42 to Aβ40 (Aβ42/40 ratio). In some embodiments, an increase in the Aβ42/40 ratio after administration of the first dose of the composition comprising the anti-Aβ protofibril antibody, e.g., an increase to a ratio of about 0.05-0.1, e.g., about 0.08-0.1, e.g., about 0.092, indicates a change from amyloid positive to amyloid negative in the brain of the subject. In some embodiments, an increase in the Aβ42/40 ratio after administration of the first dose of the composition comprising the anti-Aβ protofibril antibody, e.g., an increase to a ratio above 0.092, indicates a change from amyloid positive to amyloid negative in the brain of the subject. In some embodiments, an increase in the Aβ42/40 ratio after 6 months or after 12 months or after 18 months or after 24 months or after 36 months following the start of administration of the composition comprising the anti-Aβ protofibril antibody, e.g., an increase to a ratio of about 0.05-0.1, e.g., about 0.08-0.1, e.g., about 0.092, indicates a change from amyloid positive to amyloid negative in the brain of the subject. In some embodiments, a subject who changes to amyloid negative is given a reduced dose or frequency of the anti-Aβ protofibril antibody, alone or in combination with at least one additional therapy, e.g., a BACE inhibitor and/or anti-tau antibody.

Any of the anti-Aβ protofibril antibodies, therapeutically acceptable amounts thereof, dosing regimens therefor, and compositions comprising the same that are disclosed herein may be used in the method of converting an amyloid-positive subject to an amyloid-negative subject. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, once every month, once every five weeks, once every six weeks, once every seven weeks, once every eight weeks, once every two months, once every nine weeks, once every ten weeks, once every eleven weeks, once every twelve weeks, once every three months (quarterly), once every fourteen weeks, once every sixteen weeks, once every four months, once every eighteen weeks, once every twenty weeks, once every five months, once every 22 weeks, once every 24 weeks, once every six months (semi-annually), once every seven months, once every eight months, once every nine months, once every ten months, once every eleven months, once every twelve months (annually), once every thirteen months, once every fourteen months, once every fifteen months, once every sixteen months, once every seventeen months, or once every eighteen months.

In some embodiments, the method comprises measuring an Aβ42/40 ratio before administering a first dose of the composition comprising the anti-Aβ protofibril antibody, and measuring again after administering the antibody, e.g., after 6-12 or 18 or 24 months of treatment. In some embodiments, an increase in the Aβ42/40 ratio, e.g., to a ratio of about 0.08-0.1, e.g., about 0.092, indicates a change from amyloid positive to amyloid negative in the brain of the subject. In some embodiments, an increase in the Aβ42/40 ratio, e.g., to a ratio above 0.092, indicates a change from amyloid positive to amyloid negative in the brain of the subject. In some embodiments, a subject who changes to amyloid negative is given a reduced dose or frequency of the anti-Aβ protofibril antibody, alone or in combination with at least one additional therapy, e.g., a BACE inhibitor and/or anti-tau antibody.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, or at least 81% of the subjects being converted from amyloid positive to amyloid negative, as determined by visual reads of amyloid PET images.

In some embodiments, administration of the composition results in a conversion of 50% to 100%, such as 60% to 90%, of subjects from amyloid positive to amyloid negative, as determined by visual reads of amyloid PET images. In some embodiments, administration of the composition results in at least 55%, such as at least 60% or at least 65%, of the subjects being amyloid negative, as determined by visual reads of amyloid PET images, after 12 months of administration of the composition comprising a therapeutically effective amount of at least one anti-Aβ protofibril antibody. In some embodiments, administration of the composition results in at least 70%, such as at least 75% or at least 80%, of the subjects being amyloid negative, as determined by visual reads of amyloid PET images, after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images, wherein the subjects are ApoE4-positive.

In some embodiments, administration of the composition results in 75% to 100%, such as 80% to 100% or 85% to 100% of the subjects being amyloid negative, as determined by visual reads of amyloid PET images, wherein the subjects are ApoE4-positive. In some embodiments, administration of the composition results in at least 75%, such as at least 80% or at least 85%, of the subjects being amyloid negative, as determined by visual reads of amyloid PET images, wherein the subjects are ApoE4-positive. In some embodiments, administration of the composition results in 100% of the subjects being amyloid negative, as determined by visual reads of amyloid PET images, wherein the subjects are ApoE4-positive.

In some embodiments, at least 75%, such as at least 80% or at least 85%, of the ApoE4-positive subjects are amyloid negative, as determined by visual reads of amyloid PET images, after 12 months of administration of the composition comprising a therapeutically effective amount of at least one anti-Aβ protofibril antibody. In some embodiments, at least 75%, such as at least 80%, at least 85%, at least 90%, or at least 95%, of the ApoE4-positive subjects are amyloid negative, as determined by visual reads of amyloid PET images, after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, or at least 79% of the subjects being amyloid negative, as determined by visual reads of amyloid PET images, wherein the subjects are ApoE4-negative.

In some embodiments, administration of the composition results in 50% to 100%, such as 55% to 90%, of the subjects being amyloid negative, as determined by visual reads of amyloid PET images, wherein the subjects are ApoE4-negative. In some embodiments, administration of the composition results in at least 50% of the subjects being amyloid negative, as determined by visual reads of amyloid PET images, wherein the subjects are ApoE4-negative. In some embodiments, administration of the composition results in at least 70% of the subjects being amyloid negative, as determined by visual reads of amyloid PET images, wherein the subjects are ApoE4-negative.

### Reduction of Brain Amyloid Level

In various embodiments, also provided herein is a method of reducing brain amyloid level in a subject in need thereof. In some embodiments, the method comprises measuring an Aβ42/40 ratio before administering a first dose of a composition comprising the anti-Aβ protofibril antibody, and measuring again after administering the antibody, e.g., after 6-12 months of treatment. In some embodiments, an increase in the Aβ42/40 ratio indicates a reduction in brain amyloid in the brain of the subject. In some embodiments, a subject who exhibits a reduction in brain amyloid as determined by the change in the Aβ42/40 ratio is given a reduced dose or frequency of the anti-Aβ protofibril antibody, alone or in combination with at least one additional therapy, e.g., a BACE inhibitor and/or anti-tau antibody.

In some embodiments, the subject has early Alzheimer's disease. In some embodiments, the subject has Alzheimer's disease, Down's Syndrome, chronic traumatic encephalopathy, cerebral amyloid angiopathy, Lewy Body Dementia, or another brain disease or conditions with Aβ peptide-containing soluble and/or insoluble Aβ aggregates.

One of ordinary skill in the art will understand that, in addition to subjects having Alzheimer's disease, Aβ plaque deposits are present in the brains of subjects having other neurodegenerative diseases and conditions and thus that the methods disclosed herein may be beneficial for subjects having such neurodegenerative diseases and/or conditions. Such diseases and conditions are known to include, for example, Down's Syndrome, chronic traumatic encephalopathy, cerebral amyloid angiopathy, and Lewy Body Dementia. (See, e.g., Catafau et al., “Amyloid PET imaging: applications beyond Alzheimer's disease,” Clin. Transl. Imaging 3(1): 39-55 (2015); and Banerjee, G. et al., “The increasing impact of cerebral amyloid angiopathy: essential new insights for clinical practice,” J. Neurol. Neurosurg. Psychiatry 88: 982-994 (2017).)

In some embodiments, the subject having early Alzheimer's disease has been diagnosed as having mild cognitive impairment due to Alzheimer's disease-intermediate likelihood and/or has been diagnosed as having mild Alzheimer's disease dementia. In some embodiments, the subject having early Alzheimer's disease is ApoE4-positive.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, once every month, once every five weeks, once every six weeks, once every seven weeks, once every eight weeks, once every two months, once every nine weeks, once every ten weeks, once every eleven weeks, once every twelve weeks, once every three months (quarterly), once every fourteen weeks, once every sixteen weeks, once every four months, once every eighteen weeks, once every twenty weeks, once every five months, once every 22 weeks, once every 24 weeks, once every six months (semi-annually), once every seven months, once every eight months, once every nine months, once every ten months, once every eleven months, once every twelve months (annually), once every thirteen months, once every fourteen months, once every fifteen months, once every sixteen months, once every seventeen months, or once every eighteen months. In some embodiments, the at least one anti-Aβ protofibril antibody is BAN2401.

In some embodiments, said method results in a reduced brain amyloid level after administration relative to the brain amyloid level prior to said administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration. In some embodiments, the above-recited reductions in brain amyloid levels are determined by visual reads of amyloid PET images and are expressed as PET standard uptake value ratios (SUVr values).

As used herein, the term “PET” or “Amyloid PET” refers to Amyloid positron emission tomography imaging. In some embodiments, PET imaging (also referred to as a PET scan) is performed to assess for amyloid pathology. In some embodiments, amyloid PET is assessed with a PET tracer and uses the same tracer in follow-up assessments. In some embodiments, the PET imaging uses a florbetapir tracer. In some embodiments, the PET imaging uses a flutemetamol tracer.

Amyloid positron emission tomography (PET) imaging can be used to confirm the presence of amyloid pathology in the brain of early AD subjects in the screening phase of the study and/or to evaluate the effects of the at least one anti-AB antibody on amyloid levels in the brain, both by whole brain analysis (e.g., the average of 5-6 cortical regions) and brain region analysis. In some embodiments, the PET scan uses florbetapir. In some embodiments, amyloid plaque load can be identified by a PET imaging uptake visual read, e.g., by a trained radiologist. In some embodiments, 2 readers (1 designated as Primary Reader) visually assess the images to determine whether the scan is positive or negative for amyloid. In further embodiments, four regions of the brain are assessed for uptake of the imaging agent: the temporal lobes, the occipital lobes, the prefrontal cortex, and the parietal cortex and a positive amyloid scan has either 1 region with intense gray matter uptake that is greater than the white matter uptake and extends to the outer edges of the brain, or 2 regions with areas of reduced gray-white contrast. In further embodiments, if disagreement occurs between 2 readers, both meet to review the scan for a consensus read.

In some embodiments, amyloid plaque load can be identified by a standard uptake value ratio (SUVr) as compared to a reference region. Methods for calculating PET SUVr are known in the art and may include those described herein. In some embodiments, a Standard Uptake Value Ratio Quantitative analysis of amyloid levels is completed using PMOD Biomedical Image Quantification Software (PMOD Technologies, Zurich, Switzerland). In some embodiments, PET images are first assessed for subject movement in the X, Y, and Z planes and corrected for motion, if needed, before individual images (e.g., 5-minute emission frames) are averaged, e.g., using a PMOD Averaging Function (PET frames averaged to increase the signal to noise ratio). In some embodiments, corresponding MRIs from subjects are prepared (e.g., using matrix size reduction processing, cropping of the MRI to include only the brain, segmentation to separate images into binary maps of gray matter, white matter, and CSF, and stripping the image of skull leaving only brain mask). In some embodiments, the averaged PET images and prepared MRIs are matched using the PMOD Matching Function, placing the images in the same orientation. In some embodiments, a Brain Normalization function, e.g., as provided by PMOD software, is used along with Brain Norm and Rigid Matching transformation matrices, to produce an averaged PET. In some embodiments, this averaged PET which is normalized to the MNInst space (Senjem et al, 2005) that is in the same orientation as the subject's segmented MRI for quantitative analysis. In some embodiments, the PMOD Mask Function is used to mask the brain and zero the image outside of the mask to create a Normalized Gray Matter PET and a Normalized White Matter PET. Standard uptake values (SUVs) may be calculated for all gray matter mapped regions and the 3 white matter regions (pons, cerebellar white, and subcortical white) using PMOD software calculated using the normalized PET, subject weight, and injected dose of tracer to arrive at the units of SUVs. In some embodiments, the SUVr is the ratio of the global cortical average as compared to a reference region of choice. In some embodiments, a whole cerebellum mask is used as the reference region. In some embodiments, the reference region is subcortical white matter, derived whole cerebellum, whole cerebellum adjusted by subcortical white matter, cerebellar gray matter, and composite reference regions consisting of cerebellar cortex, pons subcortical white matter, and cerebella white matter.

In some embodiments, after administration of the first dose of the composition the adjusted mean change from baseline in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to baseline. In some embodiments, the adjusted mean change from baseline in a subject's PET SUVr value is reduced by −0.20 to −0.30.

In some embodiments, the amyloid beta plaque levels in the brain are evaluated using PET imaging. In some embodiments, the PET imaging uses a florbetapir tracer. In some embodiments, the PET imaging used a flutemetamol tracer. In some further embodiments, different tracers may yield different results. In some embodiments, the adjusted mean reduction threshold is dependent upon the tracer used. In some embodiments, comparing global cortical average versus whole cerebellum reference, the adjusted mean change from baseline in a subject's PET SUVr value is reduced by at least −0.20, such as at least −0.25, after 12 months of administration of the composition comprising a therapeutically effective amount of at least one anti-Aβ protofibril antibody. In some embodiments, the adjusted mean change from baseline in a subject's PET SUVr value is reduced by at least −0.25, such as at least −0.30, after 18 months of administration of the composition comprising a therapeutically effective amount of at least one anti-Aβ protofibril antibody.

In some embodiments, the reduction of amyloid in the brain is determined by imaging using binding of radiotracers for brain Aβ amyloid and visualized with PET. In some embodiments, the reduction in the adjusted mean change from baseline is at least −50, such as at least −55 or at least −59 centiloid after 12 months of administration of the composition comprising a therapeutically effective amount of at least one anti-Aβ protofibril antibody. In some embodiments, the reduction in the adjusted mean change from baseline is at least −60, such as at least −65 or at least −70 centiloid after 18 months of administration of the composition comprising a therapeutically effective amount of at least one anti-Aβ protofibril antibody.

In some embodiments, said method results in an increase cerebrospinal fluid Aβ 1-42 level relative to the cerebrospinal fluid Aβ1-42 level prior to said administration. In some embodiments, said method results in an increase of cerebrospinal fluid Aβ1-42 level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ1-42 level prior to said administration.

In some embodiments, administration of the composition results in a brain amyloid level reduction of −0.20 to −0.45, such as from −0.25 to −0.35 as determined by visual reads of amyloid PET images, wherein the subject is ApoE4-positive. In some embodiments, administration of the composition results in a brain amyloid level reduction of at least −0.25, as determined by visual reads of amyloid PET images, wherein the subject is ApoE4-positive. In some embodiments, administration of the composition results in a brain amyloid level reduction of at least 0.30, as determined by visual reads of amyloid PET images, wherein the subject is ApoE4-positive.

In some embodiments, a subject's brain amyloid level is determined by visual reads of amyloid PET images and expressed as a PET standard uptake value ratio (SUVr value). In some embodiments, administration of the composition results in a brain amyloid level reduction, as measured by a PET SUVr value, of at least −0.01, at least −0.02, at least −0.03, at least −0.04, at least −0.05, at least −0.06, at least −0.07, at least −0.08, at least −0.09, at least −0.10, at least −0.11, at least −0.12, at least −0.13, at least −0.14, at least −0.15, at least −0.16, at least −0.17, at least −0.18, at least −0.19, at least −0.20, at least −0.21, at least −0.22, at least −0.23, at least −0.24, at least −0.25, at least −0.26, at least −0.27, at least −0.28, or at least −0.29 relative to placebo, wherein the subject is ApoE4-negative.

In some embodiments, administration of the composition results in a brain amyloid level reduction of −0.10 to −0.40, as measured by a PET SUVr value, wherein the subject is ApoE4-negative. In some embodiments, administration of the composition results in a brain amyloid level reduction of at least −0.20, as measured by a PET SUVr value, wherein the subject is ApoE4-negative. In some embodiments, administration of the composition results in a brain amyloid level reduction of at least −0.25, as measured by a PET SUVr value, wherein the subject is ApoE4-negative.

In some embodiments, a subject's brain amyloid level is determined by visual reads of amyloid PET images and expressed as a PET standard uptake value ratio (SUVr value). In some embodiments, administration of the composition results in a brain amyloid level reduction of −0.10 to −0.40, as measured by a PET SUVr value, wherein the subject is ApoE4-negative. In some further embodiments, the subject has an increase in the Aβ42/40 ratio after administration of the first dose of the composition comprising the anti-Aβ protofibril antibody, e.g., an increase to a ratio of about 0.05-0.1, e.g., about 0.08-0.1, e.g., about 0.092, indicating a change from amyloid positive to amyloid negative in the brain of the subject. In some further embodiments, the subject has an increase in the Aβ42/40 ratio after administration of the first dose of the composition comprising the anti-Aβ protofibril antibody, e.g., an increase to a ratio above 0.092, indicating a change from amyloid positive to amyloid negative in the brain of the subject. In some embodiments, the subject has an increase in the Aβ42/40 ratio after 6 months or after 12 months or after 18 months or after 24 months of administration of the first dose of the composition comprising the anti-Aβ protofibril antibody, e.g., an increase to a ratio of about 0.05-0.1, e.g., about 0.08-0.1, e.g., about 0.092, indicating a change from amyloid positive to amyloid negative in the brain of the subject. In some embodiments, the subject has an increase in the Aβ42/40 ratio after 6 months or after 12 months or after 18 months or after 24 months of administration of the first dose of the composition comprising the anti-Aβ protofibril antibody, e.g., an increase to a ratio above 0.092, indicating a change from amyloid positive to amyloid negative in the brain of the subject. In some embodiments, an increase in the Aβ42/40 ratio indicates a reduction in brain amyloid level, as determined by visual reads of amyloid PET images. In some embodiments, a subject with a reduction in brain amyloid level is given a reduced dose or frequency of the anti-Aβ protofibril antibody, alone or in combination with at least one additional therapy, e.g., a BACE inhibitor and/or anti-tau antibody.

### Additional Biomarker Changes

### Cerebrospinal Fluid Level of Neurogranin

In some embodiments, administration of a composition comprising a therapeutically effective amount of at least one anti-Aβ protofibril antibody disclosed herein to a subject results in a reduction in cerebrospinal fluid level of neurogranin in the subject. In some embodiments, the administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, or at least 10%, relative to baseline, in cerebrospinal fluid level of neurogranin.

In some embodiments, administration to a subject of a composition comprising a therapeutically effective amount of at least one anti-Aβ protofibril antibody disclosed herein results in a reduction in cerebrospinal fluid level of neurogranin after 18 months of administration of the composition comprising a therapeutically effective amount of at least one anti-Aβ protofibril antibody. In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, or at least 10%, relative to baseline, cerebrospinal fluid level of neurogranin after 18 months of administration of the composition.

In some embodiments, administration to a subject of a composition comprising a therapeutically effective amount of at least one anti-Aβ protofibril antibody disclosed herein results in a reduction of at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, or at least about 65 μg/mL, relative to baseline, cerebrospinal fluid level of neurogranin. In some embodiments, administration to a subject of a composition comprising a therapeutically effective amount of at least one anti-Aβ protofibril antibody disclosed herein results in a reduction of at least about 65 μg/mL, relative to baseline, cerebrospinal fluid level of neurogranin.

In some embodiments, administration to a subject of a composition comprising a therapeutically effective amount of at least one anti-Aβ protofibril antibody disclosed herein results in a reduction of at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, or at least about 65 μg/mL, relative to baseline, of cerebrospinal fluid level of neurogranin after 18 months of administration of the composition comprising a therapeutically effective amount of at least one anti-Aβ protofibril antibody. In some embodiments, administering to a subject a composition comprising a therapeutically effective amount of at least one anti-Aβ protofibril antibody disclosed herein results in a reduction of at least 65 μg/mL, relative to baseline, of cerebrospinal fluid level of neurogranin after 18 months of administration of the composition.

In some embodiments, the at least one anti-Aβ protofibril antibody is BAN2401.

In some embodiments, the therapeutically effective amount of at least one anti-Aβ protofibril antibody is 10 mg/kg. In some embodiments, the composition comprising a therapeutically effective amount of at least one anti-Aβ protofibril antibody disclosed herein is administered biweekly or monthly. In some embodiments, a composition comprising 10 mg/kg of BAN2401 is administered biweekly. In some embodiments, a composition comprising 10 mg/kg of BAN2401 is administered monthly.

### Cerebrospinal Fluid Level of Neurofilament Light Chain

In some embodiments, administration to a subject of a composition comprising a therapeutically effective amount of at least one anti-Aβ protofibril antibody disclosed herein results in a reduction, relative to placebo, in cerebrospinal fluid level of neurofilament light chain. In some embodiments, administration to a subject of a composition comprising a therapeutically effective amount of at least one anti-Aβ protofibril antibody disclosed herein results in a reduction of at least 5%, at least 10%, at least 15%, at least 20%, at least 25%, at least 30%, at least 35%, at least 40%, at least 45%, or at least 50%, relative to placebo, in cerebrospinal fluid level of neurofilament light chain.

In some embodiments, administration to a subject of a composition comprising a therapeutically effective amount of at least one anti-Aβ protofibril antibody disclosed herein results in a reduction, relative to placebo, in cerebrospinal fluid level of neurofilament light chain after 18 months of administration of the composition. In some embodiments, administration to a subject of a composition comprising a therapeutically effective amount of at least one anti-Aβ protofibril antibody disclosed herein results in a reduction, relative to placebo, of at least 5%, at least 10%, at least 15%, at least 20%, at least 25%, at least 30%, at least 35%, at least 40%, at least 45%, or at least 50%, relative to baseline, in cerebrospinal fluid level of neurofilament light chain after 18 months of administration of the composition.

In some embodiments, administration to a subject of a composition comprising a therapeutically effective amount of at least one anti-Aβ protofibril antibody disclosed herein results in production of more than about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, relative to baseline, of cerebrospinal fluid level of neurofilament light chain. In some embodiments, administration to a subject of a composition comprising a therapeutically effective amount of at least one anti-Aβ protofibril antibody disclosed herein results in production of no more than about 75 μg/mL, relative to baseline, of cerebrospinal fluid level of neurofilament light chain.

In some embodiments, administration to a subject of a composition comprising a therapeutically effective amount of at least one anti-Aβ protofibril antibody disclosed herein results in production of more than about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, relative to baseline, of cerebrospinal fluid level of neurofilament light chain after 18 months of administration of the composition. In some embodiments, administration to a subject of a composition comprising a therapeutically effective amount of at least one anti-Aβ protofibril antibody disclosed herein results in production of no more than about 75 μg/mL, relative to baseline, of cerebrospinal fluid level of neurofilament light chain after 18 months of administration of the composition.

In some embodiments, the at least one anti-Aβ protofibril antibody is BAN2401.

In some embodiments, the therapeutically effective amount of at least one anti-Aβ protofibril antibody disclosed herein is 10 mg/kg. In some embodiments, a composition comprising a therapeutically effective amount of at least one anti-Aβ protofibril antibody disclosed herein is administered biweekly or monthly. In some embodiments, a composition comprising 10 mg/kg of BAN2401 is administered biweekly. In some embodiments, a composition comprising 10 mg/kg of BAN2401 is administered monthly.

### Cerebrospinal Fluid Level of Phospho-Tau

In some embodiments, administration to a subject of a composition comprising a therapeutically effective amount of at least one anti-Aβ protofibril antibody disclosed herein results in a reduction in cerebrospinal fluid level of phospho-Tau (p-tau). In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, at least 10%, at least 11%, at least 12%, or at least 13% relative to baseline, of cerebrospinal fluid level of phospho-Tau.

In some embodiments, administration to a subject of a composition comprising a therapeutically effective amount of at least one anti-Aβ protofibril antibody disclosed herein results in a reduction in cerebrospinal fluid level of phospho-Tau after 18 months of administration of the composition. In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, at least 10%, at least 11%, at least 12%, or at least 13%, relative to baseline, of cerebrospinal fluid level of phospho-Tau after 18 months of administration of the composition comprising a therapeutically effective amount of at least one anti-Aβ protofibril antibody.

In some embodiments, administration to a subject of a composition comprising a therapeutically effective amount of at least one anti-Aβ protofibril antibody disclosed herein results in a reduction of at least about 65 μg/mL, at least about 70 μg/mL, at least about 75 μg/mL, at least about 80 μg/mL, at least about 85 μg/mL, at least about 90 μg/mL, or at least about 95 μg/mL, relative to baseline, of cerebrospinal fluid level of phospho-Tau. In some embodiments, administration to a subject of a composition comprising a therapeutically effective amount of at least one anti-Aβ protofibril antibody disclosed herein results in a reduction of at least about 95 μg/mL, relative to baseline, of cerebrospinal fluid level of phospho-Tau.

In some embodiments, administration to a subject of a composition comprising a therapeutically effective amount of at least one anti-Aβ protofibril antibody disclosed herein results in a reduction of at least about 65 μg/mL, at least about 70 μg/mL, at least about 75 μg/mL, at least about 80 μg/mL, at least about 85 μg/mL, at least about 90 μg/mL, or at least about 95 μg/mL, relative to baseline, of cerebrospinal fluid level of phospho-Tau after 18 months of administration of the composition comprising a therapeutically effective amount of at least one anti-Aβ protofibril antibody. In some embodiments, administration to a subject of a composition comprising a therapeutically effective amount of at least one anti-Aβ protofibril antibody disclosed herein results in a reduction of at least 95 μg/mL, relative to baseline, of cerebrospinal fluid level of phospho-Tau after 18 months of administration of the composition comprising a therapeutically effective amount of at least one anti-Aβ protofibril antibody.

In some embodiments, the at least one anti-Aβ protofibril antibody is BAN2401.

In some embodiments, the therapeutically effective amount of at least one anti-Aβ protofibril antibody is 10 mg/kg. In some embodiments, the composition comprising a therapeutically effective amount of at least one anti-Aβ protofibril antibody disclosed herein is administered biweekly or monthly. In some embodiments, a composition comprising 10 mg/kg of BAN2401 is administered biweekly. In some embodiments, a composition comprising 10 mg/kg of BAN2401 is administered monthly.

### Brain Volume

In some embodiments, administration to a subject of a composition comprising a therapeutically effective amount of at least one anti-Aβ protofibril antibody disclosed herein results in an improvement, relative to placebo, of total hippocampal atrophy as measured by volumetric MRI (vMRI). In some embodiments, a subject's brain volume (e.g., total ventricular volume, total, right and/or left hippocampal volumes) is measured before treatment. In some embodiments, a subject's brain volume (e.g., total ventricular volume, total, right and/or left hippocampal volumes) is measured at 6 and 12 months after treatment. In some embodiments, administration to a subject of a composition comprising a therapeutically effective amount of at least one anti-Aβ protofibril antibody disclosed herein results in an improvement, relative to placebo, of brain volume atrophy as measured by vMRI.

### Treating a Subject Having Early Alzheimer's Disease Resulting in Reduction of Severity of Symptom Relative to Severity Prior to Treatment

In various embodiments, also provided herein is a method of treating a subject having early Alzheimer's disease. In some embodiments, the method comprises administering to said subject a composition comprising a therapeutically effective amount of at least one anti-Aβ protofibril antibody disclosed herein. In some embodiments, the subject having early Alzheimer's disease has been diagnosed as having mild cognitive impairment due to Alzheimer's disease-intermediate likelihood and/or has been diagnosed as having mild Alzheimer's disease dementia. In some embodiments, the subject having early Alzheimer's disease is ApoE4-positive. In some embodiments, the method comprises measuring an Aβ42/40 ratio in a subject before administering a first dose of a composition comprising the anti-Aβ protofibril antibody, and measuring again after administering the antibody, e.g., after 6-12 months of treatment. In some embodiments, an increase in the Aβ42/40 ratio indicates a reduction in the severity of at least one symptom associated with Alzheimer's disease, wherein the severity of at least one symptom associated with Alzheimer's disease is reduced by at least 10%, 20%, 30%, 40%, 50%, 60%, 70%, 80%, 90%, or 95% relative to the severity of the same symptom in the same subject prior to treatment. In some embodiments, the severity of the at least one symptom associated with Alzheimer's disease is determined by ADCOMS, PET, MMSE, CDR-SB, and/or ADAS-Cog. In some embodiments, the at least one symptom associated with Alzheimer's disease is chosen from clinical decline and brain amyloid level.

In some embodiments, the severity of at least one symptom associated with Alzheimer's disease is reduced by at least 1%. In some embodiments, the severity of at least one symptom associated with Alzheimer's disease is reduced by at least 10%. In some embodiments, the severity of at least one symptom associated with Alzheimer's disease is reduced by at least 20%. In some embodiments, the severity of at least one symptom associated with Alzheimer's disease is reduced by at least 30%. In some embodiments, the severity of at least one symptom associated with Alzheimer's disease is reduced by at least 40%. In some embodiments, the severity of at least one symptom associated with Alzheimer's disease is reduced by at least 50%. In some embodiments, the severity of at least one symptom associated with Alzheimer's disease is reduced by at least 60%. In some embodiments, the severity of at least one symptom associated with Alzheimer's disease is reduced by at least 70%. In some embodiments, the severity of at least one symptom associated with Alzheimer's disease is reduced by at least 80%. In some embodiments, the severity of at least one symptom associated with Alzheimer's disease is reduced by at least 90%. In some embodiments, the severity of at least one symptom associated with Alzheimer's disease is reduced by at least 95%.

In some embodiments, the above-recited reduction in severity is determined after 1 month, 6 months, 12 months, 18 months, 60 months, and/or 63 months of administration of the composition comprising a therapeutically effective amount of at least one anti-Aβ protofibril antibody.

In some embodiments, a subject with a reduction in brain amyloid as determined by an increased Aβ42/40 ratio exhibits a reduction in the severity of at least one symptom associated with Alzheimer's disease. In some embodiments, a subject with an increased Aβ42/40 ratio is given a reduced dose or frequency of the anti-Aβ protofibril antibody, alone or in combination with at least one additional therapy, e.g., a BACE inhibitor and/or anti-tau antibody.

Prevention and/or Delay of Onset of Alzheimer's Disease

In various embodiments, also provided herein is a method of preventing and/or delaying onset of Alzheimer's disease, e.g., in ApoE4-positive subjects. In some embodiments, said method comprises determining the brain amyloid level of a subject and then, if the brain amyloid level of the subject is above a first predetermined level, administering a composition comprising a therapeutically effective amount of at least one anti-Aβ protofibril antibody.

In some embodiments, the subject is pre-symptomatic (cognitively unimpaired) and is selected for treatment (e.g., treatment comprising at least one anti-Aβ protofibril antibody such as BAN2401) based on a reduced Aβ42/40 ratio in a blood (e.g., plasma) sample relative to the ratio in a blood sample from a healthy subject or an average ratio from a population of such healthy subjects. In some embodiments, the pre-symptomatic subject is ApoE4-positive. In some embodiments, treatment prevents or delays the onset of AD symptoms. In some embodiments, the subject is aged 55-80.

In some embodiments, the method further comprises measuring the post-administration brain amyloid level of the subject. In some embodiments, treatment increase the Aβ42/40 ratio in a blood sample relative to the level in a sample from the subject prior to treatment comprising the anti-Aβ protofibril antibody. In some embodiments, treatment is continued if an increase in the Aβ42/40 ratio relative to the level in a sample from the subject prior to treatment comprising the anti-Aβ protofibril antibody is observed.

In various embodiments, one or more additional biomarker is measured to select a pre-symptomatic subject for treatment and/or to monitor treatment efficacy.

In some embodiments, brain amyloid levels are measured by PET in a subject in conjunction with measuring the Aβ42/40 ratio in a blood sample from the subject. In some embodiments, brain amyloid levels are not measured by PET (e.g., to reduce cost or increase speed of screening). In some embodiments, the Aβ42/40 ratio in a blood sample from a subject is measured with or without the brain amyloid measurement by PET prior to the treatment for screening or selecting a subject to be treated with lecanemab. In some embodiments, the method further comprises determining a cerebrospinal fluid level of Aβ1-42 and/or cerebrospinal fluid total tau level. In some embodiments, a total tau level in blood is measured. In some embodiments, a p-tau level in blood is measured, e.g., one or more of p-181 or p-217 tau.

In some embodiments, the method further comprises determining a cerebrospinal fluid level of neurogranin.

In some embodiments, the method further comprises determining a cerebrospinal fluid level of neurofilament light chain.

In some embodiments, said method further comprises administering the composition if the post-administration brain amyloid level is above a second predetermined level.

In some embodiments, said method further comprises monitoring the brain amyloid level of the subject after administration until the brain amyloid level of the subject is below a first predetermined level.

In some embodiments, said method further comprises administering the composition if the post-administration cerebrospinal fluid level of Aβ1-42 and/or cerebrospinal fluid total tau level is above a predetermined level.

In some embodiments, said method further comprises administering the composition if the post-administration cerebrospinal fluid level of neurogranin is above a predetermined level.

In some embodiments, said method further comprises administering the composition if the post-administration neurofilament light chain is above a predetermined level.

In some embodiments, said method further comprises administering at least one additional therapeutic agent. In some embodiments, the at least one additional therapeutic agent is chosen from BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof. In some embodiments, the at least one additional therapeutic agent is a BACE inhibitor. In some embodiments, the BACE inhibitor is chosen from CNP520, BI-1181181, LY2886721, LY3202626, PF-06751979, RG7129, atabecestat, elenbecestat, lanabecestat, and verubecestat. In some embodiments, the BACE inhibitor is elenbecestat.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, once every month (“mo”), once every five weeks, once every six weeks, once every seven weeks, once every eight weeks, once every two months, once every nine weeks, once every ten weeks, once every eleven weeks, once every twelve weeks, once every three months (quarterly), once every fourteen weeks, once every sixteen weeks, once every four months, once every eighteen weeks, once every twenty weeks, once every five months, once every 22 weeks, once every 24 weeks, once every six months (semi-annually), once every seven months, once every eight months, once every nine months, once every ten months, once every eleven months, once every twelve months (annually), once every thirteen months, once every fourteen months, once every fifteen months, once every sixteen months, once every seventeen months, or once every eighteen months.

In some embodiments, the at least one anti-Aβ protofibril antibody is BAN2041.

In some embodiments, administration of the composition comprising at least one anti-Aβ protofibril antibody results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one anti-Aβ protofibril antibody results in 75% to 100%, such as 80% to 100% or 85% to 100% of the subjects being amyloid negative, as determined by visual reads of amyloid PET images. In some embodiments, administration of the composition comprising at least one anti-Aβ protofibril antibody results in at least 75%, such as at least 80% or at least 85%, of the subjects being amyloid negative, as determined by visual reads of amyloid PET images. In some embodiments, administration of the composition comprising at least one anti-Aβ protofibril antibody results in 100% of the subjects being amyloid negative, as determined by visual reads of amyloid PET images.

In some embodiments, at least 75%, such as at least 80% or at least 85%, of the subjects are amyloid negative, as determined by visual reads of amyloid PET images, after 12 months of administration of the composition comprising a therapeutically effective amount of at least one anti-Aβ protofibril antibody. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the method results in a reduced brain amyloid level after administration relative to the brain amyloid level prior to the administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration. In some embodiments, the above-recited reductions in brain amyloid levels are determined by visual reads of amyloid PET images and are expressed as PET standard uptake value ratios (SUVr values).

In some embodiments, the adjusted mean change in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to the brain amyloid level prior to administration of the composition comprising at least one anti-Aβ protofibril antibody. In some embodiments, the adjusted mean change in a subject's PET SUVr value from the brain amyloid level prior to the administration of the composition comprising at least one anti-Aβ protofibril antibody is reduced by −0.20 to −0.30.

In some embodiments, comparing global cortical average versus whole cerebellum reference, the adjusted mean change from the brain amyloid level prior to the administration of the composition comprising at least one anti-Aβ protofibril antibody in a subject's PET SUVr value is reduced by at least −0.20, such as at least −0.25, after 12 months of administration of the composition comprising a therapeutically effective amount of at least one anti-Aβ protofibril antibody. In some embodiments, the adjusted mean change in a subject's PET SUVr value is reduced by at least −0.25, such as at least −0.30, relative to the subject's PET SUVr value prior to the administration, after 18 months of administration of the composition comprising a therapeutically effective amount of at least one anti-Aβ protofibril antibody.

In some embodiments, the reduction of amyloid in the brain is determined by imaging using binding of radiotracers for brain Aβ amyloid and visualized with PET. In some embodiments, the reduction in the adjusted mean change from the subject's level prior to the administration of the composition comprising a therapeutically effective amount of at least one anti-Aβ protofibril antibody is at least −50, such as at least −55 or at least −59 centiloid after 12 months of administration of the composition comprising a therapeutically effective amount of at least one anti-Aβ protofibril antibody. In some embodiments, the reduction in the adjusted mean change from the subject's level prior to the administration of the composition comprising a therapeutically effective amount of at least one anti-Aβ protofibril antibody is at least −60, such as at least −65 or at least −70 centiloid after 18 months of administration of the composition comprising a therapeutically effective amount of at least one anti-Aβ protofibril antibody.

In some embodiments, the method results in an increased cerebrospinal fluid Aβ1-42 level relative to the cerebrospinal fluid Aβ1-42 level prior to the administration. In some embodiments, the method results in an increased of cerebrospinal fluid Aβ1-42 level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ1-42 level prior to the administration.

In some embodiments, administration of the composition comprising at least one anti-Aβ protofibril antibody results in a brain amyloid level reduction of −0.20 to −0.45, such as from −0.25 to −0.35 as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody results in a brain amyloid level reduction of at least −0.25, as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody results in a brain amyloid level reduction of at least 0.30, as determined by visual reads of amyloid PET images, relative to the brain amyloid level prior to administration of the composition comprising at least one anti-Aβ protofibril antibody.

Also provided herein is another method of preventing and/or delaying onset Alzheimer's disease in ApoE4-positive subjects. In some embodiments, said method comprises determining the brain amyloid level of a subject and then, if the brain amyloid level of the subject is above a first predetermined level, administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody.

In some embodiments, the method further comprises measuring the post-administration brain amyloid level of the subject.

In some embodiments, the method further comprises determining a cerebrospinal fluid level of Aβ1-42 and/or cerebrospinal fluid total tau level.

In some embodiments, the method further comprises determining a cerebrospinal fluid level of neurogranin.

In some embodiments, the method further comprises determining a cerebrospinal fluid level of neurofilament light chain.

In some embodiments, said method further comprises administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody if the post-administration brain amyloid level is above a second predetermined level.

In some embodiments, said method further comprises administering the composition if the post-administration cerebrospinal fluid level of Aβ1-42 and/or cerebrospinal fluid total tau level is above a predetermined level.

In some embodiments, said method further comprises administering the composition if the post-administration cerebrospinal fluid level of neurogranin is above a predetermined level.

In some embodiments, said method further comprises administering the composition if the post-administration cerebrospinal fluid level of neurofilament light chain is above a predetermined level.

In some embodiments, said method further comprises monitoring the brain amyloid level of the subject after administration until the brain amyloid level of the subject is below a first predetermined level.

In some embodiments, said method further comprises administering at least one additional therapeutic agent. In some embodiments, the at least one additional therapeutic agent is chosen from BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof.

In some embodiments, the at least one additional therapeutic agent is a BACE inhibitor. In some embodiments, the BACE inhibitor is elenbecestat.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, once every month (“mo”), once every five weeks, once every six weeks, once every seven weeks, once every eight weeks, once every two months, once every nine weeks, once every ten weeks, once every eleven weeks, once every twelve weeks, once every three months (quarterly), once every fourteen weeks, once every sixteen weeks, once every four months, once every eighteen weeks, once every twenty weeks, once every five months, once every 22 weeks, once every 24 weeks, once every six months (semi-annually), once every seven months, once every eight months, once every nine months, once every ten months, once every eleven months, once every twelve months (annually), once every thirteen months, once every fourteen months, once every fifteen months, once every sixteen months, once every seventeen months, or once every eighteen months.

In some embodiments, the at least one anti-Aβ protofibril antibody is BAN2041.

In some embodiments, the at least one therapeutic agent is a BACE inhibitor. In some embodiments, the BACE inhibitor is elenbecestat.

In some embodiments, administration of the composition comprising at least one anti-Aβ protofibril antibody and the composition comprising a therapeutically effective amount of at least one therapeutic agent results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one anti-Aβ protofibril antibody and the composition comprising a therapeutically effective amount of at least one therapeutic agent results in 75% to 100%, such as 80% to 100% or 85% to 100% of the subjects being amyloid negative, as determined by visual reads of amyloid PET images. In some embodiments, administration of the composition comprising at least one anti-Aβ protofibril antibody and the composition comprising a therapeutically effective amount of at least one therapeutic agent results in at least 75%, such as at least 80% or at least 85%, of the subjects being amyloid negative, as determined by visual reads of amyloid PET images. In some embodiments, administration of the composition comprising at least one anti-Aβ protofibril antibody and the composition comprising a therapeutically effective amount of at least one therapeutic agent results in 100% of the subjects being amyloid negative, as determined by visual reads of amyloid PET images.

In some embodiments, at least 75%, such as at least 80% or at least 85%, of the subjects are amyloid negative, as determined by visual reads of amyloid PET images, after 12 months of administration of the composition comprising a therapeutically effective amount of at least one anti-Aβ protofibril antibody and the composition comprising a therapeutically effective amount of at least one therapeutic agent. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one anti-Aβ protofibril antibody and the composition comprising a therapeutically effective amount of at least one therapeutic agent. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the method results in a reduced brain amyloid level after administration relative to the brain amyloid level prior to the administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration. In some embodiments, the above-recited reductions in brain amyloid levels are determined by visual reads of amyloid PET images and are expressed as PET standard uptake value ratios (SUVr values).

In some embodiments, the adjusted mean change in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to the brain amyloid level prior to administration of the composition comprising at least one anti-Aβ protofibril antibody and the composition comprising a therapeutically effective amount of at least one therapeutic agent. In some embodiments, the adjusted mean change in a subject's PET SUVr value from the brain amyloid level prior to the administration of the composition comprising at least one anti-Aβ protofibril antibody and the composition comprising a therapeutically effective amount of at least one therapeutic agent is reduced by −0.20 to −0.30.

In some embodiments, comparing global cortical average versus whole cerebellum reference, the adjusted mean change from the brain amyloid level prior to the administration of the composition comprising at least one anti-Aβ protofibril antibody and the composition comprising a therapeutically effective amount of at least one therapeutic agent in a subject's PET SUVr value is reduced by at least −0.20, such as at least −0.25, after 12 months of administration of the composition comprising a therapeutically effective amount of at least one anti-Aβ protofibril antibody and the composition comprising a therapeutically effective amount of at least one therapeutic agent. In some embodiments, the adjusted mean change in a subject's PET SUVr value is reduced by at least −0.25, such as at least −0.30, relative to the subject's PET SUVr value prior to the administration, after 18 months of administration of the composition comprising a therapeutically effective amount of at least one anti-Aβ protofibril antibody and the composition comprising a therapeutically effective amount of at least one therapeutic agent.

In some embodiments, the reduction of amyloid in the brain is determined by imaging using binding of radiotracers for brain Aβ amyloid and visualized with PET. In some embodiments, the reduction in the adjusted mean change from the subject's level prior to the administration of the composition comprising a therapeutically effective amount of at least one anti-Aβ protofibril antibody and the composition comprising a therapeutically effective amount of at least one therapeutic agent is at least −50, such as at least −55 or at least −59 centiloid after 12 months of administration of the composition comprising a therapeutically effective amount of at least one anti-Aβ protofibril antibody and the composition comprising a therapeutically effective amount of at least one therapeutic agent. In some embodiments, the reduction in the adjusted mean change from the subject's level prior to the administration of the composition comprising a therapeutically effective amount of at least one anti-Aβ protofibril antibody is at least −60, such as at least −65 or at least −70 centiloid after 18 months of administration of the composition comprising a therapeutically effective amount of at least one anti-Aβ protofibril antibody and the composition comprising a therapeutically effective amount of at least one therapeutic agent.

In some embodiments, the method results in an increased cerebrospinal fluid Aβ1-42 level relative to the cerebrospinal fluid Aβ1-42 level prior to the administration. In some embodiments, the method results in an increased of cerebrospinal fluid Aβ1-42 level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ1-42 level prior to the administration.

In some embodiments, administration of the composition comprising at least one anti-Aβ protofibril antibody and the composition comprising a therapeutically effective amount of at least one therapeutic agent results in a brain amyloid level reduction of −0.20 to −0.45, such as from −0.25 to −0.35 as determined by visual reads of amyloid PET images, relative to the brain amyloid level prior to administration of the composition comprising at least one anti-Aβ protofibril antibody and the composition comprising a therapeutically effective amount of at least one therapeutic agent. In some embodiments, administration of the composition comprising at least one anti-Aβ protofibril antibody and the composition comprising a therapeutically effective amount of at least one therapeutic agent results in a brain amyloid level reduction of at least −0.25, as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody and the composition comprising a therapeutically effective amount of at least one therapeutic agent results in a brain amyloid level reduction of at least 0.30, as determined by visual reads of amyloid PET images, relative to the brain amyloid level prior to administration of the composition comprising at least one anti-Aβ protofibril antibody and the composition comprising a therapeutically effective amount of at least one therapeutic agent.

### EXEMPLARY EMBODIMENTS

1. A method of treating Alzheimer's disease (AD) in a subject having or suspected of having AD, comprising
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-amyloid β (Aβ) protofibril antibody;
d. measuring the concentration of Aβ42 and Aβ40 in a second blood sample obtained from the subject after the first sampling to determine a second Aβ42/40 ratio;
e. optionally, measuring a second level of p-tau181 in the second blood sample obtained from the subject;
and
f. administering a second therapeutically effective dose comprising the same or a lower amount of the anti-Aβ protofibril antibody than in the first dose to the subject having i) an elevated second ratio relative to the first ratio and/or ii) a second level of p-tau181 that is lower than the first level of p-tau181.

2. A method of treating AD in a subject having or suspected of having AD, comprising
a. measuring a concentration of Aβ42 and a concentration of Aβ40 in a first blood sample obtained from the subject to determine a first ratio of Aβ42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-Aβ protofibril antibody;
d. measuring the concentration of Aβ42 and Aβ40 in a second blood sample obtained from the subject after the first sampling to determine a second Aβ42/40 ratio;
e. optionally, measuring a second level of p-tau181 in the second blood sample obtained from the subject;
and
f. administering a second therapeutically effective dose comprising a higher amount of the anti-Aβ protofibril antibody than in the first dose or a different treatment for AD to the subject having i) the same or a reduced second ratio relative to the first ratio and/or ii) a second level of p-tau181 that is the same as or higher than the first level of p-tau181.

3. The method of embodiment 1 or embodiment 2, wherein the subject has AD.

4. The method of embodiment 1 or embodiment 2, wherein the subject has been diagnosed with early AD.

5. The method of embodiment 1 or embodiment 2, wherein the subject has been diagnosed as having mild cognitive impairment due to Alzheimer's disease-intermediate likelihood and/or has been diagnosed as having mild Alzheimer's disease dementia.

6. The method of embodiment 5, wherein the subject has been diagnosed with mild cognitive impairment due to Alzheimer's disease-intermediate likelihood by National Institute of Aging-Alzheimer's Association (NIA-AA) core clinical criteria.

7. The method of embodiment 5, wherein the subject has been diagnosed with mild cognitive impairment due to Alzheimer's disease-intermediate likelihood by a CDR global score of 0.5 and a Memory Box score of 0.5 or greater before treatment.

8. The method of embodiment 5, wherein the subject has been diagnosed with mild cognitive impairment due to Alzheimer's disease-intermediate likelihood by a history of subjective memory decline with gradual onset and slow progression over the last 1 year before treatment, e.g., as corroborated by an informant.

9. The method of embodiment 5, wherein the subject has been diagnosed with mild Alzheimer's disease dementia by the NIA-AA core clinical criteria for probable Alzheimer's disease dementia.

10. The method of embodiment 5, wherein the subject has been diagnosed with mild Alzheimer's disease dementia by a CDR score of 0.5 to 1.0 and a Memory Box score of 0.5 or greater before treatment.

11. The method of any one of the embodiments 1-10, wherein the subject is amyloid-positive prior to administration, e.g., as indicated by a PET assessment, a CSF assessment of Aβ(1-42), MRI, retinal amyloid accumulation, and/or specific behavioral/cognitive phenotypes.

12. The method of any one of the embodiments 1-11, wherein the subject has at least one copy of the ApoE4 gene.

13. The method of any one of the embodiments 1-12, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).

14. The method of embodiment 13, wherein the anti-Aβ protofibril antibody comprises a heavy chain variable region comprising an amino acid sequence of SEQ ID NO: 7 and a light chain variable region comprising an amino acid sequence of SEQ ID NO: 8.

15. The method of embodiment 13 or 14, wherein the anti-Aβ protofibril antibody comprises a heavy chain comprising an amino acid sequence of SEQ ID NO: 9 and a light chain comprising an amino acid sequence of SEQ ID NO: 10.

16. The method of any one of the embodiments 1-15, wherein the second sample is obtained at least 1 week, 2 weeks, 3 weeks, 1 month, 2 months, 3 months, 6 months, 12 month, 18 months or 24 months after the first sample.

17. The method of any one of the embodiments 1-16, wherein the anti-Aβ protofibril antibody is administered as an infusion.

18. The method of any one of the embodiments 1-17, wherein the Aβ42/40 ratio is measured using an LC MS/MS platform.

19. The method of any one of the embodiments 1-18, wherein the first therapeutically effective dose of the anti-Aβ protofibril antibody comprises 2.5 mg/kg to 15 mg/kg relative to the weight of the subject, e.g., about 10 mg/kg.

20. The method of any one of the embodiments 1-19, wherein the therapeutically effective dose of the anti-Aβ protofibril antibody comprises 10 mg/kg relative to the weight of the subject, wherein the dose is administered once every 2 weeks.

21. The method according to embodiment 19, wherein the composition is administered once every 2 weeks or once every month.

22. The method of any one of the embodiments 1 or 3-21, wherein the frequency of administration is reduced e.g., to monthly, bimonthly, quarterly, or semi-annual administration, in a subject having an elevated second Aβ42/40 ratio relative to the first Aβ42/40 ratio and/or a second level of p-tau181 that is lower than the first level of p-tau181.

23. The method of any one of the embodiments 2-21, wherein the frequency of administration is increased in a subject having a reduced second Aβ42/40 ratio relative to the first Aβ42/40 ratio and/or a second level of p-tau181 that is the same as or higher than the first level of p-tau181.

24. The method any one of the embodiments 1 or 3-22, wherein the elevated second Aβ42/40 ratio relative to the first Aβ42/40 ratio indicates that the treatment has converted the subject from amyloid-positive to amyloid-negative.

25. The method of embodiment 1, wherein the amount of the anti-Aβ protofibril antibody in the second therapeutically effective dose is reduced, relative to the amount in the first therapeutically effective dose and/or the frequency of administration is reduced, e.g., from biweekly to monthly or to every six months.

26. The method of embodiment 1, wherein the second ratio is elevated by at least 10% relative to the first ratio, e.g., when measured at least 6 months after a first therapeutically effective dose of the anti-Aβ protofibril antibody, and/or wherein the ratio is increased to at least about 0.092.

27. The method of any one of the embodiments 1-26, wherein the subject is sequentially or simultaneously administered at least one additional AD medication.

28. The method any one of the embodiments 1-27, wherein the second therapeutically effective dose is administered in combination with a second therapeutic.

29. The method of any one of the embodiments 1-28, wherein the method results in a reduction of a cerebrospinal fluid level of Aβ1-42, total tau, phospho-tau, and/or neurogranin, and/or results in a slowing of increase in cerebrospinal fluid level of neurofilament light chain as compared to before treatment.

30. The method of any one of the embodiments 1-29, further comprising measuring brain amyloid level in the subject before and/or after treatment, e.g., by measuring a PET SUVr value.

31. The method of embodiment 30, comprising administering the second therapeutically effective dose of the anti-Aβ protofibril antibody if a reduction in brain amyloid level is detected, e.g., as determined by an adjusted mean reduction from baseline in a PET SUVr value of at least about 0.10, or 0.15, or 0.20, e.g., after 6 or 12 months of treatment with the first therapeutically effective dose.

32. The method of any one of the embodiments 1-31, wherein the method reduces brain amyloid level in the subject, as measured by an adjusted mean change from baseline in a PET SUVr value.

33. The method of embodiment 32, wherein the adjusted mean change from baseline in the PET SUVr value is a reduction of at least about 0.10, or 0.15, or 0.20, e.g., after 12 months of treatment with the first therapeutically effective dose.

34. The method of any one of the embodiments 1-33, wherein the treatment delays clinical decline as determined by ADCOMS.

35. The method of any one of the embodiments 1-33, wherein the treatment delays clinical decline as determined by ADAS MCI-ADL.

36. The method of any one of the embodiments 1-33, wherein the treatment delays clinical decline as determined by modified iADRS.

37. The method of any one of the embodiments 1-33, wherein the treatment delays clinical decline as measured by a CDR-SB.

38. The method of any one of the embodiments 1-33, wherein the treatment delays clinical decline as measured by an ADAS-Cog.

39. The method of any one of the embodiments 1-38, wherein the method of treatment further comprises monitoring for ARIA, e.g., ARIA-E and/or ARIA-H, e.g., as observed by MRI.

40. The method of any one of the embodiments 1 or 3-39, wherein the method reduces a risk of ARIA, e.g., ARIA-E and/or ARIA-H, e.g., as observed by MRI.

41. The method of embodiment 40, wherein ARIA-E does not increase in size or number after treatment, e.g., as measured by MRI.

42. The method of embodiment 40, wherein ARIA-H does not increase in size or number after treatment, e.g., as measured by MRI.

43. A method of reducing brain amyloid beta in a subject having or suspected of having AD, comprising
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-amyloid β (Aβ) protofibril antibody;
d. measuring the concentration of amyloid β 1-42 (Aβ42) and amyloid β 1-40 (Aβ40) in a second blood sample obtained from the subject after the first sampling to determine a second Aβ42/40 ratio;
e. optionally, measuring a second level of p-tau181 in the second blood sample obtained from the subject;
and
f. administering a second therapeutically effective dose comprising the same or a lower amount of the anti-Aβ protofibril antibody than in the first dose to the subject having i) an elevated second ratio relative to the first ratio and/or ii) a second level of p-tau181 that is lower than the first level of p-tau181,
thereby reducing brain amyloid beta in the subject.
44. A method of reducing brain amyloid beta in a subject having or suspected of having AD, comprising
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. optionally, measuring a first level of phosphorylated tau181 (p-tau181) in the first blood sample obtained from the subject;
c. administering to the subject a first therapeutically effective dose of an anti-amyloid β (Aβ) protofibril antibody;
d. measuring the concentration of amyloid β 1-42 (Aβ42) and amyloid β 1-40 (Aβ40) in a second blood sample obtained from the subject after the first sampling to determine a second Aβ42/40 ratio;
e. optionally, measuring a second level of p-tau181 in the second blood sample obtained from the subject;
and
f. administering a second therapeutically effective dose comprising a higher amount of the anti-Aβ protofibril antibody than in the first dose to the subject having i) the same or a reduced second ratio relative to the first ratio and/or ii) a second level of p-tau181 that is the same as or higher than the first level of p-tau181,
thereby reducing brain amyloid beta in the subject.

45. The method of embodiment 43 or embodiment 44, wherein the subject has AD.

46. The method of embodiment 43 or embodiment 44, wherein the subject has been diagnosed with early AD.

47. The method of embodiment 43 or embodiment 44, wherein the subject has been diagnosed as having mild cognitive impairment due to Alzheimer's disease-intermediate likelihood and/or has been diagnosed as having mild Alzheimer's disease dementia.

48. The method of embodiment 47, wherein the subject has been diagnosed with mild cognitive impairment due to Alzheimer's disease-intermediate likelihood by National Institute of Aging-Alzheimer's Association (NIA-AA) core clinical criteria.

49. The method of embodiment 47, wherein the subject has been diagnosed with mild cognitive impairment due to Alzheimer's disease-intermediate likelihood by a CDR global score of 0.5 and a Memory Box score of 0.5 or greater before treatment.

50. The method of embodiment 47, wherein the subject has been diagnosed with mild cognitive impairment due to Alzheimer's disease-intermediate likelihood by a history of subjective memory decline with gradual onset and slow progression over the last 1 year before treatment, e.g., as corroborated by an informant.

51. The method of embodiment 57, wherein the subject has been diagnosed with mild Alzheimer's disease dementia by the NIA-AA core clinical criteria for probable Alzheimer's disease dementia.

52. The method of embodiment 57, wherein the subject has been diagnosed with mild Alzheimer's disease dementia by a CDR score of 0.5 to 1.0 and a Memory Box score of 0.5 or greater before treatment.

53. The method of any one of the embodiments 43-52, wherein the subject is amyloid-positive prior to administration, e.g., as indicated by a PET assessment, a CSF assessment of Aβ(1-42), MRI, retinal amyloid accumulation, and/or specific behavioral/cognitive phenotypes.

54. The method of any one of the embodiments 43-53, wherein the subject has at least one copy of the ApoE4 gene.

55. The method of any one of the embodiments 43-54, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).

56. The method of embodiment 55, wherein the anti-Aβ protofibril antibody comprises a heavy chain variable region comprising an amino acid sequence of SEQ ID NO: 7 and a light chain variable region comprising an amino acid sequence of SEQ ID NO: 8. 557. The method of embodiment 55 or 56, wherein the anti-Aβ protofibril antibody comprises a heavy chain comprising an amino acid sequence of SEQ ID NO: 9 and a light chain comprising an amino acid sequence of SEQ ID NO: 10.

58. The method of any one of the embodiments 43-57, wherein the second sample is obtained at least 1 week, 2 weeks, 3 weeks, 1 month, 2 months, 3 months, 6 months, 12 month, 18 months or 24 months after the first sample.

59. The method of any one of the embodiments 43-58, wherein the anti-Aβ protofibril antibody is administered as an infusion.

60. The method of any one of the embodiments 43-59, wherein the Aβ42/40 ratio is measured using an LC MS/MS platform.

61. The method of any one of the embodiments 43-60, wherein the first therapeutically effective dose of the anti-Aβ protofibril antibody comprises 2.5 mg/kg to 15 mg/kg relative to the weight of the subject, e.g., about 10 mg/kg.

62. The method of any one of the embodiments 43-61, wherein the therapeutically effective dose of the anti-Aβ protofibril antibody comprises 10 mg/kg relative to the weight of the subject, wherein the dose is administered once every 2 weeks.

63. The method according to embodiment 61, wherein the composition is administered once every 2 weeks or once every month.

64. The method of any one of the embodiments 43 or 45-63, wherein the frequency of administration is reduced e.g., to monthly, bimonthly, quarterly, or semi-annual administration in a subject having an elevated second Aβ42/40 ratio relative to the first Aβ42/40 ratio and/or a second level of p-tau181 that is lower than the first level of p-tau181.

65. The method of any one of the embodiments 44-63, wherein the frequency of administration is increased in a subject having a reduced second Aβ42/40 ratio relative to the first Aβ42/40 ratio and/or a second level of p-tau181 that is the same or higher than the first level of p-tau181.

66. The method any one of the embodiments 43 or 45-64, wherein the elevated second Aβ42/40 ratio relative to the first Aβ42/40 ratio indicates that the treatment has converted the subject from amyloid-positive to amyloid-negative.

67. The method of embodiment 43, wherein the amount of the anti-Aβ protofibril antibody in the second therapeutically effective dose is reduced, relative to the amount in the first therapeutically effective dose and/or the frequency of administration is reduced, e.g., from biweekly to monthly or to every six months.

68. The method any one of the embodiments 43-67, wherein the second therapeutically effective dose is administered in combination with a second therapeutic.

69. The method of embodiment 43, wherein the second ratio is elevated by at least 10% relative to the first ratio, e.g., when measured at least 6 months after a first therapeutically effective dose of the anti-Aβ protofibril antibody, and/or wherein the ratio is increased to at least about 0.092.

70. The method of any one of the embodiments 43-69, wherein the subject is sequentially or simultaneously administered at least one additional AD medication.

71. The method of any one of the embodiments 43-70, wherein the method results in a reduction of a cerebrospinal fluid level of Aβ1-42, total tau, phospho-tau, and/or neurogranin, and/or results in a slowing of increase in cerebrospinal fluid level of neurofilament light chain as compared to before treatment.

72. The method of any one of the embodiments 43-71, further comprising measuring brain amyloid level in the subject before and/or after treatment, e.g., by measuring a PET SUVr value.

73. The method of embodiment 72, comprising administering the second therapeutically effective dose of the anti-Aβ protofibril antibody if a reduction in brain amyloid level is detected, e.g., as determined by an adjusted mean reduction from baseline in a PET SUVr value of at least about 0.10, or 0.15, or 0.20, e.g., after 6 or 12 months of treatment with the first therapeutically effective dose.

74. The method of any one of the embodiments 43-73, wherein the method reduces brain amyloid level in the subject, as measured by an adjusted mean change from baseline in a PET SUVr value.

75. The method of embodiment 74, wherein the adjusted mean change from baseline in the PET SUVr value is a reduction of at least about 0.10, or 0.15, or 0.20, e.g., after 12 months of treatment with the first therapeutically effective dose.

76. The method of any one of the embodiments 43-75, wherein the treatment delays clinical decline as determined by ADCOMS.

77. The method of any one of the embodiments 43-76, wherein the treatment delays clinical decline as determined by ADAS MCI-ADL.

78. The method of any one of the embodiments 43-77, wherein the treatment delays clinical decline as determined by modified iADRS.

79. The method of any one of the embodiments 43-78, wherein the treatment delays clinical decline as measured by a CDR-SB.

80. The method of any one of the embodiments 43-79, wherein the treatment delays clinical decline as measured by an ADAS-Cog.

81. The method of any one of the embodiments 43-80, wherein the method of treatment further comprises monitoring for ARIA, e.g., ARIA-E and/or ARIA-H, e.g., as observed by MRI.

82. The method of any one of the embodiments 43 or 45-81, wherein the method reduces a risk of ARIA, e.g., ARIA-E and/or ARIA-H, e.g., as observed by MRI.

83. The method of embodiment 82, wherein ARIA-E does not increase in size or number after treatment, e.g., as measured by MRI.

84. The method of embodiment 82, wherein ARIA-H does not increase in size or number after treatment, e.g., as measured by MRI.

85. A method of monitoring treatment efficacy in a subject having or suspected of having AD, comprising:
a. administering to the subject a therapeutically effective dose of an anti-Aβ protofibril antibody;
b. measuring the concentration of Aβ42 and Aβ40 in a blood sample obtained from the subject to determine a Aβ42/40 ratio;
c. optionally, measuring the level of phosphorylated tau181 (p-tau181) in the blood sample;
d. comparing the Aβ42/40 ratio of the sample to a ratio in a sample from the patient prior to treatment or in a control, wherein the same or a higher Aβ42/40 ratio after treatment indicates an effective treatment; and
e. optionally, comparing the level of p-tau181 in a sample from the patient prior to treatment or in a control, wherein a reduced level of p-tau181 after treatment indicates an effective treatment.

86. The method of embodiment 85, wherein the same or a higher Aβ42/40 ratio after treatment indicates a reduction in a brain Aβ level.

87. The method of embodiment 85, wherein a reduced level of p-tau181 after treatment indicates a reduction in a brain Aβ level.

88. The method of embodiment 85, further comprising measuring brain amyloid level in the subject before and/or after treatment, e.g., by measuring a PET SUVr value.

89. The method of embodiment 88, wherein the adjusted mean change from baseline in the PET SUVr value is a reduction of at least about 0.10, or 0.15, or 0.20, e.g., after 12 months of treatment with the first therapeutically effective dose.

90. The method of embodiment 88, wherein the treatment efficacy is confirmed by a visual read of PET images to confirm the subject is amyloid-negative.

91. A method of detecting a decrease in a brain Aβ level, comprising:
a. measuring a concentration of Aβ42 and a concentration of Aβ40 in a first blood sample obtained from the subject to determine a first ratio of Aβ 42 to Aβ40 (Aβ42/40 ratio) prior to treatment;
b. optionally, measuring a level of phosphorylated tau181 (p-tau181) in the first blood sample obtained from the subject prior to treatment;
c. measuring the concentration of Aβ42 and Aβ40 in a second blood sample obtained from the subject after the first sampling to determine a second Aβ42/40 ratio after administering a therapeutically effective dose of an anti-Aβ protofibril antibody;
d. optionally, measuring the level of p-tau181 in the second blood sample obtained from the subject after the first sampling to determine a second level after administering a therapeutically effective dose of an anti-Aβ protofibril antibody;
e. comparing the first and second ratios, wherein an elevated Aβ42/40 ratio in the second sample relative to the first sample indicates a decrease of brain amyloid β in the subject at the second sampling relative to the first sampling; and
f. optionally, comparing the first and second levels, wherein a reduced level in the second sample relative to the first sample indicates a decrease of brain amyloid β in the subject at the second sampling relative to the first sampling.

92. A method of reducing brain amyloid level in a subject in need thereof comprising:
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. optionally, measuring a level of phosphorylated tau181 (p-tau181) in the first blood sample obtained from the subject to obtain a first level of p-tau181;
c. administering to the subject a first therapeutically effective dose of an anti-amyloid β (Aβ) protofibril antibody;
d. measuring the concentration of Aβ42 and Aβ40 in a second blood sample obtained from the subject after the first sampling to determine a second Aβ42/40 ratio;
e. optionally, measuring a level of p-tau181 in the second blood sample to determine a second level;
and
f. administering a second therapeutically effective dose comprising the same or a lower amount of the anti-Aβ protofibril antibody than in the first dose to the subject having i) an elevated second ratio relative to the first ratio and/or ii) an reduced second level of p-tau181 relative to the first level.

93. The method according to embodiment 92, wherein the subject has Alzheimer's disease, Down's Syndrome, chronic traumatic encephalopathy, cerebral amyloid angiopathy, Lewy Body Dementia, or another brain disease or conditions with Aβ peptide-containing soluble and/or insoluble Aβ aggregates.

94. The method of embodiment 92 or embodiment 93, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).

95. The method of embodiment 94, wherein the anti-Aβ protofibril antibody comprises a heavy chain variable region comprising an amino acid sequence of SEQ ID NO: 7 and a light chain variable region comprising an amino acid sequence of SEQ ID NO: 8.

96. The method of embodiment 94 or 95, wherein the anti-Aβ protofibril antibody comprises a heavy chain comprising an amino acid sequence of SEQ ID NO: 9 and a light chain comprising an amino acid sequence of SEQ ID NO: 10.

97. The method of any one of the embodiments 1-96, wherein the subject is administered a maintenance dose.

98. The method of embodiment 97, wherein the maintenance dose comprises an anti-amyloid β (Aβ) protofibril antibody

99. The method of embodiment 98, wherein the maintenance dose is administered once or multiple times.

100. The method of embodiment 99, wherein the maintenance dose is administered at a dose frequency selected to maintain a PET SUVr level achieved during treatment.

101. The method of embodiment 99, wherein the maintenance dose is administered at a dose frequency selected to maintain a PET SUVr level at or below amyloid negativity (e.g. for florbetapir, PET SUVr of 1.17).

102. The method of any one of the embodiments 99-101, wherein the maintenance dose is administered every three months at a dosage of 10 mg/kg.

103. The method of any one of embodiments 99-101, wherein the maintenance dose is administered every month at a dosage of 10 mg/kg.

104. The method of embodiment 99, wherein the maintenance dose is administered at a dose frequency selected to maintain a Aβ42/40 ratio and/or a p-tau181 level achieved during treatment.

105. The method of embodiment 104, wherein the maintenance dose is administered at a dose frequency selected to maintain a Aβ42/40 ratio at or above 0.092.

106. The method of any one of embodiments 104-105, wherein the maintenance dose is administered every month at a dosage of 10 mg/kg.

107. The method of embodiment 98 or 99, wherein the maintenance dose is administered at a lower dose than during an earlier course of treatment.

108. The method of embodiment 98 or 99, wherein the maintenance dose is administered less frequently than during the earlier course of treatment.

109. The method of embodiment 99, wherein the maintenance dose is administered weekly, biweekly, monthly, or every 3 months.

110. The method of embodiment 99, wherein the maintenance dose is administered after a subject obtains a reduced Aβ42/40 ratio, e.g., a ratio below about 0.092-0.094.

111. The method of any one of the embodiments 97-99, wherein the maintenance dose comprises 2.5 mg/kg to 15 mg/kg relative to the weight of the subject.

112. The method of embodiment 111, wherein the maintenance dose comprises 10 mg/kg.

113. A method of selecting a subject for treatment with an anti-amyloid β (Aβ) protofibril antibody, comprising
a. measuring a concentration of amyloid β 1-42 (Aβ42) and a concentration of amyloid β 1-40 (Aβ40) in a blood sample obtained from the subject to determine a ratio of Aβ42 to Aβ40 (Aβ42/40 ratio); and
b. selecting the subject for treatment if the ratio is below a threshold (e.g., about 0.092-0.094).

114. A method of treating Alzheimer's disease (AD) in a subject having or suspected of having AD, comprising
a. measuring a concentration of amyloid β 1-42 (Aβ42) and a concentration of amyloid β 1-40 (Aβ40) in a blood sample obtained from the subject to determine a ratio of Aβ42 to Aβ40 (Aβ42/40 ratio); and
b. administering a treatment comprising a therapeutically effective dose of an anti-amyloid β (Aβ) protofibril antibody to the subject having an Aβ42/40 ratio below a threshold (e.g., of about 0.092-0.094).

115. The method of any one of the embodiments 1-111, wherein administering to the subject a first therapeutically effective dose of an anti-Aβ protofibril antibody does not require a titration step.

116. A method of treating Alzheimer's disease (AD) in a subject having or suspected of having AD, comprising administering to the subject a treatment comprising a therapeutically effective dose of an anti-amyloid β (Aβ) protofibril antibody biweekly for a period of time sufficient to achieve a brain amyloid negativity as measured by PET SUVr, e.g., a florbetapir PET SUVr level at or below 1.17, then administering a maintenance dose of the antibody at least every three months to maintain a PET SUVr level at or below amyloid negativity (e.g. for florbetapir, PET SUVr of 1.17).

117. The method of embodiment 116, wherein the antibody is administered at a dosage of 10 mg/kg body weight of the subject.

118. The method of embodiment 116 or 117, wherein the maintenance dose is administered monthly.

119. The method of any one of the embodiments 97-118, wherein the subject has early AD.

120. The method of any one of the embodiments 97-118, wherein the subject has pre-AD.

121. The method of any one of the embodiments 1-120, wherein an Aβ42/40 ratio is measured in a blood sample from the subject prior to treatment.

122. The method of any one of the embodiments 1-121, wherein a p-tau181 level is measured in a blood sample from the subject prior to treatment.

123. The method of embodiment 121 or 122, wherein the Aβ42/40 ratio is measured without a brain amyloid measurement by PET.

124. A method of treating Alzheimer's disease (AD) in a subject having or suspected of having AD, comprising
a. measuring a concentration of amyloid β 1-42 (Aβ42) and a concentration of amyloid β 1-40 (Aβ40) in a first blood sample obtained from the subject to determine a first ratio of Aβ 42 to Aβ40 (Aβ42/40 ratio);
b. administering to the subject a first therapeutically effective dose of an anti-amyloid β (Aβ) protofibril antibody;
c. measuring the concentration of Aβ42 and Aβ40 in a second blood sample obtained from the subject after the first sampling to determine a second Aβ42/40 ratio; and
d. administering a second therapeutically effective dose comprising the same or a lower amount of the anti-Aβ protofibril antibody than in the first dose to the subject having an elevated second ratio relative to the first ratio.

125. A method of treating AD in a subject having or suspected of having AD, comprising
a. measuring a concentration of Aβ42 and a concentration of Aβ40 in a first blood sample obtained from the subject to determine a first ratio of Aβ42 to Aβ40 (Aβ42/40 ratio);
b. administering to the subject a first therapeutically effective dose of an anti-Aβ protofibril antibody;
c. measuring the concentration of Aβ42 and Aβ40 in a second blood sample obtained from the subject after the first sampling to determine a second Aβ42/40 ratio; and
d. administering a second therapeutically effective dose comprising a higher amount of the anti-Aβ protofibril antibody than in the first dose or a different treatment for AD to the subject having the same or a reduced second ratio relative to the first ratio.

### Sequence Tables

TABLE 1
Amino acid sequences of monoclonal antibody (mAb) CDRs

| mAb | IgG chain | SEQ ID NO | Amino acid sequence |
|---|---|---|---|
| BAN2401 | HCDR1 | 1 | SFGMH |
| | HCDR2 | 2 | YISSGSSTIYYGDTVKG |
| | HCDR3 | 3 | EGGYYYGRSYYTMDY |
| BAN2401 | LCDR1 | 4 | RSSQSIVHSNGNTYLE |
| | LCDR2 | 5 | KVSNRFS |
| | LCDR3 | 6 | FQGSHVPPT |

TABLE 2
Amino acid sequences of mAb variable regions

| mAb | IgG chain | SEQ ID NO | Amino acid sequence |
|---|---|---|---|
| BAN2401 | Heavy chain | 7 | EVQLVESGGGLVQPGGSLRLSCSASGF |
| | variable | | TFSSFGMHWVRQAPGKGLEWVAYISS |
| | region | | GSSTIYYGDTVKGRFTISRDNAKNSLFL |
| | | | QMSSLRAEDTAVYYCAREGGYYYGRS |
| | | | YYTMDYWGQGTTVTVSS |
| BAN2401 | Light chain | 8 | DVVMTQSPLSLPVTPGAPASISCRSSQSI |
| | variable | | VHSNGNTYLEWYLQKPGQSPKLLIYKV |
| | region | | SNRFSGVPDRFSGSGSGTDFTLRISRVE |
| | | | AEDVGIYYCFQGSHVPPTFGPGTKLEIK |

TABLE 3
Amino acid sequences of mAb heavy and light chains

| mAb | IgG chain | SEQ ID NO | Amino acid sequence |
|---|---|---|---|
| BAN2401 | Heavy chain | 9 | EVQLVESGGGLVQPGGSLRLSCSASG |
| | | | FTFSSFGMHWVRQAPGKGLEWVAYI |
| | | | SSGSSTIYYGDTVKGRFTISRDNAKNS |
| | | | LFLQMSSLRAEDTAVYYCAREGGYY |
| | | | YGRSYYTMDYWGQGTTVTVSSASTK |
| | | | GPSVFPLAPSSKSTSGGTAALGCLVK |
| | | | DYFPEPVTVSWNSGALTSGVHTFPAV |
| | | | LQSSGLYSLSSVVTVPSSSLGTQTYICN |
| | | | VNHKPSNTKVDKRVEPKSCDKTHTCPP |
| | | | CPAPELLGGPSVFLFPPKPKDTLMISRTP |
| | | | EVTCVVVDVSHEDPEVKFNWYVDGV |
| | | | EVHNAKTKPREEQYNSTYRVVSVLT |
| | | | VLHQDWLNGKEYKCKVSNKALPAPI |
| | | | EKTISKAKGQPREPQVYTLPPSREEM |
| | | | TKNQVSLTCLVKGFYPSDIAVEWESN |
| | | | GQPENNYKTTPPVLDSDGSFFLYSKL |
| | | | TVDKSRWQQGNVFSCSVMHEALHN |
| | | | HYTQKSLSLSPGK |
| BAN2401 | Light chain | 10 | light chain: |
| | | | DVVMTQSPLSLPVTPGAPASISCRSSQ |
| | | | SIVHSNGNTYLEWYLQKPGQSPKLLI |
| | | | YKVSNRFSGVPDRFSGSGSGTDFTLRI |
| | | | SRVEAEDVGIYYCFQGSHVPPTFGPGT |
| | | | KLEIKRTVAAPSVFIFPPSDEQLKSGTAS |
| | | | VVCLLNNFYPREAKVQWKVDNALQSG |
| | | | NSQESVTEQDSKDSTYSLS |
| | | | STLTLSKADYEKHKVYACEVTHQGL |
| | | | SSPVTKSFNRGEC |

TABLE 4
Amino acid sequences of mAb constant regions

| mAb | IgG chain | Class | SEQ ID NO | Amino acid sequence |
|---|---|---|---|---|
| BAN2401 | Heavy chain | IgG1 | 11 | ASTKGPSVFPLAPSSKSTSGGT |
| | | | | AALGCLVKDYFPEPVTVSWN |
| | | | | SGALTSGVHTFPAVLQSSGLY |
| | | | | SLSSVVTVPSSSLGTQTYICNV |
| | | | | NHKPSNTKVDKRVEPKSCDK |
| | | | | THTCPPCPAPELLGGPSVFLFP |
| | | | | PKPKDTLMISRTPEVTCVVVD |
| | | | | VSHEDPEVKFNWYVDGVEVH |
| | | | | NAKTKPREEQYNSTYRVVSVL |
| | | | | TVLHQDWLNGKEYKCKVSNK |
| | | | | ALPAPIEKTISKAKGQPREPQV |
| | | | | YTLPPSREEMTKNQVSLTCLV |
| | | | | KGFYPSDIAVEWESNGQPENN |
| | | | | YKTTPPVLDSDGSFFLYSKLT |
| | | | | VDKSRWQQGNVFSCSVMHEA |
| | | | | LHNHYTQKSLSLSPGK |
| BAN2401 | Light chain | kappa | 12 | RTVAAPSVFIFPPSDEQLKSGT |
| | | | | ASVVCLLNNFYPREAKVQWK |
| | | | | VDNALQSGNSQESVTEQDSK |
| | | | | DSTYSLSSTLTLSKADYEKHK |
| | | | | VYACEVTHQGLSSPVTKSFNR |
| | | | | GEC |

TABLE 5
Amino acid sequences of Amyloid β

| Amyloid β | SEQ ID NO | Amino acid sequence |
|---|---|---|
| Amyloid β 1-42 | 13 | DAEFRHDSGYEVHHQKLVFFAEDVGSNKGAIIGL |
| | | MVGGVVIA |
| Amyloid β 1-40 | 14 | DAEFRHDSGYEVHHQKLVFFAEDVGSNKGAIIGL |
| | | MVGGVV |

TABLE 11
Amino acid sequences of E2814 CDRs

| mAb | IgG chain | SEQ ID NO | Amino acid sequence |
|---|---|---|---|
| E2814 | HCDR1 | 15 | TYWIT |
| Heavy Chain | HCDR2 | 16 | DIYPGSSISNYNEKFKS |
| | HCDR3 | 17 | EDGYDAWFAY |
| E2814 Light | LCDR1 | 18 | RSSQSILHSNGNTYLE |
| Chain | LCDR2 | 19 | KVSNRFS |
| | LCDR3 | 20 | FQGSHVPFT |

TABLE 12
Amino acid sequences of E2814 variable regions

| mAb | IgG chain | SEQ ID NO | Amino acid sequence |
|---|---|---|---|
| E2814 | Heavy chain | 21 | EVQLLESGGGLVQPGGSLRLSCAASGY |
| | variable | | TFTTYWITWVRQAPGKGLEWVSDIYPG |
| | region | | SSISNYNEKFKSRFTISVDNSKNTLYLQ |
| | | | MNSLRAEDTAVYYCAREDGYDAWFA |
| | | | YWGQGTLVTVSS |
| E2814 | Light chain | 22 | DIQMTQSPSSLSASVGDRVTITCRSSQSI |
| | variable | | LHSNGNTYLEWYQQKPGKAPKLLISKV |
| | region | | SNRFSGVPSRFSGSGSGTDFTLTISSLQP |
| | | | EDFATYYCFQGSHVPFTFGQGTKLEIK |

TABLE 13
Amino acid sequences of E2814 constant regions

| mAb | IgG chain | Class | SEQ ID NO | Amino acid sequence |
|---|---|---|---|---|
| E2814 | Heavy chain | IgG1 | 23 | ASTKGPSVFPLAPSSKSTSGGT |
| | | | | AALGCLVKDYFPEPVTVSWN |
| | | | | SGALTSGVHTFPAVLQSSGLY |
| | | | | SLSSVVTVPSSSLGTQTYICNV |
| | | | | NHKPSNTKVDKKVEPKSCDK |
| | | | | THTCPPCPAPELLGGPSVFLFP |
| | | | | PKPKDTLMISRTPEVTCVVVD |
| | | | | VSHEDPEVKFNWYVDGVEVH |
| | | | | NAKTKPREEQYNSTYRVVSVL |
| | | | | TVLHQDWLNGKEYKCKVSNK |
| | | | | ALPAPIEKTISKAKGQPREPQV |
| | | | | YTLPPSRDELTKNQVSLTCLV |
| | | | | KGFYPSDIAVEWESNGQPENN |
| | | | | YKTTPPVLDSDGSFFLYSKLT |
| | | | | VDKSRWQQGNVFSCSVMHEA |
| | | | | LHNHYTQKSLSLSPGK |
| E2814 | Light chain | kappa | 24 | RTVAAPSVFIFPPSDEQLKSGT |
| | | | | ASVVCLLNNFYPREAKVQWK |
| | | | | VDNALQSGNSQESVTEQDSK |
| | | | | DSTYSLSSTLTLSKADYEKHK |
| | | | | VYACEVTHQGLSSPVTKSFNR |
| | | | | GEC |

TABLE 14
Amino acid sequence of Tau

| | SEQ ID NO | Amino acid sequence |
|---|---|---|
| Tau | 25 | MAEPRQEFEVMEDHAGTYGLGDRKDQGGYTMHQDQEGDTDAGLKES |
| | | PLQTPTEDGSEEPGSETSDAKSTPTAEDVTAPLVDEGAPGKQAAAQPHT |
| | | EIPEGTTAEEAGIGDTPSLEDEAAGHVTQARMVSKSKDGTGSDDKKAK |
| | | GADGKTKIATPRGAAPPGQKGQANATRIPAKTPPAPKTPPSSGEPPKSG |
| | | DRSGYSSPGSPGTPGSRSRTPSLPTPPTREPKKVAVVRTPPKSPSSAKSRL |
| | | QTAPVPMPDLKNVKSKIGSTENLKHQPGGGKVQIINKKLDLSNVQSKC |
| | | GSKDNIKHVPGGGSVQIVYKPVDLSKVTSKCGSLGNIHHKPGGGQVEV |
| | | KSEKLDFKDRVQSKIGSLDNITHVPGGGNKKIETHKLTFRENAKAKTDH |
| | | GAEIVYKSPVVSGDTSPRHLSNVSSTGSIDMVDSPQLATLADEVSASLA |
| | | KQGL |

### EXAMPLES

The present disclosure is further illustrated by the following examples that should not be construed as limiting. The contents of all references, patents, and published patent applications cited throughout this application, as well as the figures, are incorporated herein by reference in their entirety for all purposes.

1. Treatment of Subjects Having Early Alzheimer's Disease with BAN2401 Lecanemab

BAN2401-G000-201 (Study 201 Core, NCT01767311) is a double-blind, parallel-group, placebo-controlled, multicenter and multinational study that utilized a dose-finding response adaptive randomization (RAR) design to evaluate the safety, tolerability, and efficacy of BAN2401 in subjects with MCI due to AD-intermediate likelihood, or with mild AD dementia (collectively designated as early AD in this study). 854 subjects were randomized for treatment. MCI due to AD-intermediate likelihood and mild AD dementia are defined by the National Institute of Aging-Alzheimer's Association (NIA-AA) core clinical criteria.

The core study consisted of a Prerandomization Phase (Screening Period and Baseline Period), and a Randomization Phase with a planned 18-month treatment period followed by a 3-month Follow-Up Period. An Open-Label Extension Phase was implemented to allow for up to 60 months (5 years) of additional treatment. There was an intervening gap period off-treatment between the core study and the Open-Label Extension ranging from 9-59 months (mean 24 months).

### Core Study

### Prerandomization Phase

The Prerandomization Phase lasted up to 60 days, and consisted of a Screening

Period (up to 30 days duration) and a Baseline Period (up to 30 days duration). During the Screening Period and Baseline Period, all subjects were assessed for eligibility using clinical tests, safety MRIs, and amyloid PET assessments to confirm that subjects meet the diagnostic criteria for MCI due to AD-intermediate likelihood or mild Alzheimer's disease dementia and that they do not have other medical conditions that may interfere with study participation. All subjects were confirmed amyloid positive via amyloid positron emission tomography (PET) or cerebrospinal fluid (CSF) Aβ1-42 for eligibility.

Further assessments were conducted during the Baseline Period, including additional clinical evaluations, mandatory blood sampling for pharmacogenomics (APOE4 status), and CSF sampling for those who consent to soluble CSF biomarker analysis. At the Baseline Visit, clinical assessments: MMSE, CDR, ADAS-Cog, and FAQ, were conducted.

### Randomization Phase

Subjects were randomized to receive placebo or 1 of 5 doses of BAN2401 (2.5, 5, or 10 mg/kg given biweekly, or 5 or 10 mg/kg given every 4 weeks [monthly]), administered by intravenous (IV) infusion for the duration of the Randomization Phase (18 months).

There was a notable protocol amendment during the course of the study related to a safety observation for apolipoprotein E4 (ApoE4) gene carriers receiving the highest dose (10 mg/kg biweekly) of lecanemab. Emerging data from the study just prior to the 350 subject interim analysis indicated that ApoE4 positive homozygous individuals on the highest dose of lecanemab had the highest risk of developing symptomatic amyloid-related imaging abnormalities-edema/effusion (ARIA-E). Following comprehensive data review, one regulatory authority requested that ApoE4 carriers (homozygous and heterozygous; approximately 70% of the overall subject population) no longer be administered the 10 mg/kg biweekly dose of lecanemab going forward, and this approach was adopted for all subsequent randomizations. At the same time, a request was also made to discontinue from study drug administration, without exception, all ApoE4 carriers (homozygous and heterozygous) who were randomized to the 10 mg/kg biweekly dose and were on study for less than 6 months.

### Efficacy Assessments

Primary efficacy of BAN2401 was assessed via Bayesian analysis, by comparing to placebo, the change from Baseline at 12 months on ADCOMS, a composite clinical score representing a new approach to the analysis of selected items (12 total) from 3 fully validated and well-established clinical tools, including CDR (all 6 items), ADAS-Cog14 (4 items), and MMSE (2 items).

The key secondary efficacy of BAN2401 was assessed using mixed model repeated measures (MMRM) analysis, by comparing to placebo at 18 months as follows: brain amyloid pathophysiology as measured by PET; clinical status on ADCOMS, CDR-SB, and ADAS-Cog14; CSF biomarkers (including Aβ[1-42], t-tau, and p-tau); information from measurements of potential novel emerging CSF biomarkers at 18 months [e.g., neurogranin and neurofilament light chain (NfL, also measured in plasma)]; and total hippocampal volume as measured by vMRI.

The secondary efficacy was assessed using MMRM analyses at 12 months using brain amyloid pathophysiology by PET, clinical status on ADCOMS, CDR-SB, and ADAS-Cog14, and CSF biomarkers (including Aβ[1-42], t-tau, and p-tau); information from measurements of potential novel emerging CSF biomarkers at 12 months [e.g., neurogranin, and NfL (also measured in plasma)]; and total hippocampal atrophy by vMRI at 6 and 12 months, and left and right hippocampus, whole brain, and total ventricular volume by vMRI at 6, 12, and 18 months.

Exploratory efficacy was evaluated by clinical status on ADCOMS, CDR-SB, and ADAS-Cog14 at other time points, as well as MMSE and FAQ.

### Amyloid PET and CSF Aβ(1-42)

Amyloid PET imaging or CSF Aβ(1-42) assessment was used to confirm that all subjects with EAD have amyloid deposition in the brain. This criterion allowed for the definition of subjects with MCI due to AD-intermediate likelihood and confirmed amyloid pathology in mild Alzheimer's disease dementia subjects.

Amyloid PET or CSF Aβ(1-42) assessment was required at Baseline during the Prerandomization Phase for all subjects to qualify for study inclusion per the protocol, and subjects who consented to participate in the imaging subgroup received amyloid PET imaging at 12 and 18 months of treatment. Duration of the PET scan and its timing relative to injection of the imaging agent were as per the imaging agent manufacturer's guidance. As the imaging agent, florbetapir and flutemetamol were used, however, most of the subjects who had amyloid PET scan used florbetapir. Data are included for those subjects who received florbetapir as the imaging agent.

Amyloid plaque load identified by PET imaging uptake was determined via 2 separate methodologies: visual read and standard uptake value ratio (SUVr) using a cortical composite versus a reference region.

### Sampling of Blood for Exploratory Plasma Biomarkers

Blood was collected from subjects at Baseline during the Prerandomization Phase prior to amyloid PET assessment and at 12 and 18 months of treatment to evaluate potential novel biomarkers of AD.

### Open-Label Extension Phase (Study 201 OLE)

The ongoing Extension Phase is conducted in accordance with the protocol outlined as below.

The Open-Label Extension (OLE) Phase was initiated following the Core Study to allow subjects to receive open-label BAN2401 10 mg/kg biweekly. All subjects who are continuing on in the Extension Phase and who have completed at least 18 months of treatment in the Extension Phase may take part in an optional dosing regimen substudy to evaluate the effects on safety, PK exposure, biomarker, and clinical efficacy of alternate dosing regimens for maintenance dosing of BAN2401. Subjects may choose to enter this substudy at any study visit according to their prior biweekly schedule of assessments. Subjects who choose to participate in this substudy will be randomized to 1 of 2 intravenous dosing regimens; either BAN2401 10 mg/kg once every 4 weeks (Q4W) or BAN2401 10 mg/kg once every 3 months (Q3M)]. All subjects will continue participation in the Extension Phase for up to 60 months (5 years), until the drug is commercially available in the country where the subject resides, or until the benefit to risk ratio from treatment with BAN2401 is no longer considered favorable, whichever comes first. Any subject who completed study treatment (Visit 42 [Week 79] of the Core Study) and fulfills the Extension Phase inclusion and exclusion criteria had the option to participate. Subjects who previously completed the Core Study (through the Follow-Up Visit, Visit 43) at any time before implementation of the Extension Phase, and/or fulfill the Extension Phase inclusion and exclusion criteria, were eligible to participate. Subjects who discontinued the Core Study were eligible to participate in the Extension Phase, provided they met the inclusion and exclusion criteria for the Extension Phase. No subjects entered the OLE Phase immediately after the Core Study completed, and for all subjects there was a gap of at least 9 months between the last dose in the Core Study and the first dose in the OLE Phase. No treatment was given, and no data were collected during this gap period.

All subjects in the ongoing extension phase have received and will continue to receive the BAN2401 10 mg/kg biweekly dose (or Q4W or Q3M, if enrolled in the dosing regimen substudy), including subjects who are confirmed APOE4 positive (hetero- or homozygous). All infusions take place in the clinic; however, if approved by the Sponsor and allowable and conducted according to country and local guidelines, subjects are offered the option of home infusions for approved visits. Home infusions are being implemented as a result of the Coronavirus Disease 2019 (COVID-19) pandemic to allow subjects who cannot visit clinical sites for various reasons to continue receiving treatment. Home infusions are allowed per Sponsor approval according to country and local guidelines during the COVID-19 pandemic and following its resolution, where permitted. The Follow-up Visit in the Extension Phase takes place 3 months after the last dose of study drug. Subjects may discontinue from study drug for any reason. Subjects who discontinue the study drug are requested to undertake the Early Termination Visit (within 7 days after the last dose of study drug) and the Follow-up Visit (3 months after the last dose of study drug).

### Efficacy Assessment

ADCOMS: This composite clinical score represents a new approach to the analysis of selected items (12 total) from 3 fully validated and well-established clinical tools, including the MMSE, the CDR, and the ADAS-Cog. The data from 4 studies, including the Alzheimer's Disease Neuroimaging Initiative (ADNI) (MCI subset), ADCS-008, E2020-A001-412 and E2020-E033-415 have been used in a statistically validated model aimed at optimizing sensitivity to disease progression over time in the MCI population. The MMSE, the CDR, and the ADAS-Cog will each be administered to subjects using standard methods, and results will be used to calculate the ADCOMS.

### Amyloid PET

All subjects who underwent amyloid PET for inclusion in the Core Study received a baseline amyloid PET scan before dosing in the Extension Phase. The baseline amyloid PET scan had to be conducted with the same imaging tracer that was used for inclusion at the baseline visit for the Core Study. In addition, qualified subjects located in the US and Japan had the option to participate in the longitudinal PET substudy. Florbetapir was used in the US and flutemetamol was used in Japan for longitudinal amyloid PET analysis. At Extension Screening Visits, subjects who consented to the longitudinal imaging substudy were stratified into 2 cohorts based on their treatment allocation during the Core Study. Cohort 1 amyloid PET assessments are performed at baseline (Extension Screening Visit), Visit 50 (Extension Week 13), Extension Phase Visit 70 [Extension Week 53], and continue annually; Cohort 2 amyloid PET assessments are performed at baseline (Extension Screening Visit), Visit 57 (Extension Week 27), at Extension Phase Visit 70 [Extension Week 53], and continue annually. In Japan, those who consented to the longitudinal imaging substudy only underwent amyloid PET at Extension Phase Visit 70 [Extension Week 53], and continue annually.

### Sampling of Blood for Exploratory Plasma Biomarkers

Blood was collected from subjects at baseline (Extension Screening Visit), Extension Phase Visit 50 [Extension Week 13], Extension Phase Visit 57 [Extension Week 27], Extension Phase Visit 70 [Extension Week 53], Extension Phase Visit 83 [Extension Week 79], Extension Phase Visit 96 [Extension Week 105], Extension Phase Visit 109 [Extension Week 131], Extension Phase Visit 122 [Extension Week 157], Extension Phase Visit 135 [Extension Week 183], Extension Phase Visit 148 [Extension Week 209], Extension Phase Visit 161 [Extension Week 235], Extension Phase Visit 174 [Extension Week 261], and early termination visit.

In the dosing regimen substudy, subjects will undertake a baseline plasma blood draw upon entry to the substudy and will attend site visits every 4 weeks, regardless of dosing regimen for the 1st year of the dosing regimen substudy. Blood will be drawn at each dosing regimen substudy visit for plasma biomarker monitoring to assess that baseline levels (at substudy entry) are maintained over the course of treatment at each dosing regimen. The sponsor will assess the plasma biomarker responses periodically, and if an optimal regimen is established, that regimen will be administered to all subjects in the substudy. For those subjects who are also enrolled in the optional longitudinal PET substudy, a dosing regimen substudy baseline amyloid PET assessment should be conducted before substudy Visit 1 (Week 1), unless it has been conducted within 3 months of the dosing regimen substudy Visit 1.

2. Correlation Between Plasma Amyloid Beta 1-42/1-40 Ratio and Brain Amyloid Beta During the Treatment with Lecanemab

The amyloid PET sub-study in the Core study assessed baseline, 12 months and 18 months SUVr with florbetapir, and participants in the OLE amyloid PET sub-study were imaged at baseline, 3 or 6 months, and 12 months. Plasma samples were collected at the same timepoints. Plasma Aβ40 and Aβ42 levels (pg/mL), and a Aβ42/40 concentration ratio were measured using a PrecivityAD™ LC MS/MS platform (C2N Diagnostics, LLC)(Kirmess, et. al., Clinica Chimica Acta 519 (2021) 267-275, West et al, Mol Neurodegen (2021) 16-30). Mean changes from Core or OLE baseline and Pearson correlation coefficients were calculated at the group and individual levels for amyloid PET SUVr and plasma Aβ 42/40 ratio, accounting for repeated measures. Baseline and demographic characteristics are provided in the table below.

Analyses on change in plasma Aβ42/40 ratio vs. dose and change in plasma Aβ42/40 ratio vs. change in PET SUVr were performed on the following data.
Subjects/data:
   Only subjects enrolled in the OLE who reached OLE 12 m and had minimum core baseline plasma samples were included (N=121 subjects)
      N=88 had at least one visual read by Florbetapir
      N=81 had at least one PET SUVr by Florbetapir
   Plasma sample analysis (835 samples from 7 timepoints in each of the 121 subjects):
      Core: Baseline, 12 m, 18,
      OLE: Baseline, 3 m, 6 m, 12 m
   Analysis populations:
      Plasma Biomarker Analysis Set: Defined as the group of subjects who had plasma Aβ42/40 ratios determined at core baseline and at least 1 post core assessment

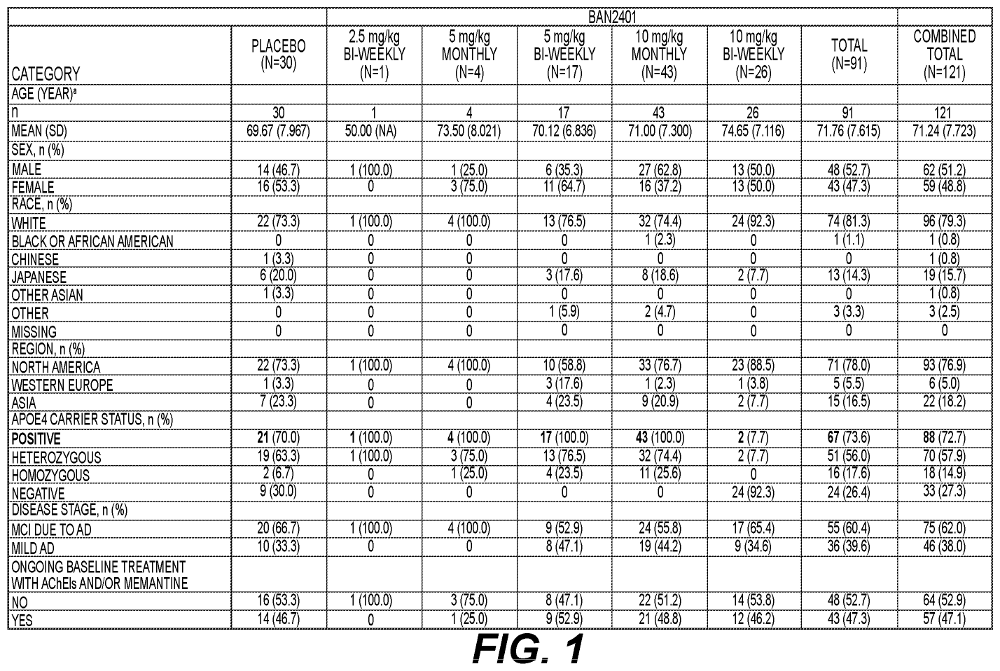

FIG. 1 is an interim summary table of patient demographics and dosing regimen.

Analyses with additional patient data: Table 6 is a summary of the patient demographics for patients in the Study 201 Core used in the full dataset analysis. Table 8 is a summary of the biomarker results in the Study 201 Core. Table 7 is a summary of the patient demographics for patients in the OLE Phase used in the full dataset analysis. Table 9 is a summary of the biomarker results in the OLE Phase. Table 10 is a summary of the clinical results in the Study 201 Core.

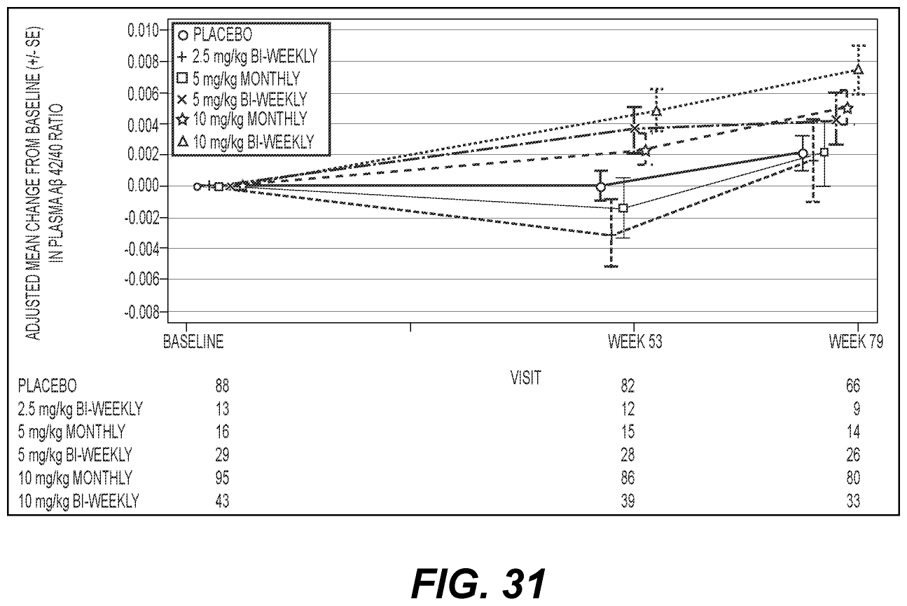

For Core study, plasma Aβ42/40 was evaluated from 284 subjects (placebo: n=88, lecanemab 2.5 mg/kg biweekly: n=13, lecanemab 5 mg/kg monthly: n=16, lecanemab 5 mg/kg biweekly: n=29, lecanemab 10 mg/kg monthly: n=95, lecanemab 10 mg/kg biweekly: n=43) (FIG. 31). The C2N PrecivityAD assay for Aβ42/40 ratio requires 500 μL of plasma sample volume. This sample volume was available only for a limited number of subjects at Baseline and postbaseline assessments; therefore, the sample size was smaller for the Aβ42/40 ratio analyses.

TABLE 6
Patient demographics for patients in the Study 201 Core used in the full dataset analysis

| | | | | Lecanemab | | | | | |
|---|---|---|---|---|---|---|---|---|---|
| | | | | 2.5 mg/kg | | 5 mg/kg | | 5 mg/kg | |
| | | Placebo | | Biweekly | | Monthly | | Biweekly | |
| Category | | (N = 238) | | (N = 52) | | (N = 48) | | (N = 89) | |
| Age (year)^{a} | n | 238 | | 52 | | 48 | | 89 | |
| | Mean (SD) | 71.11 | (8.892) | 70.50 | (8.257) | 70.42 | (7.514) | 70.64 | (7.446) |
| | Median | 72.00 | | 70.50 | | 71.00 | | 72.00 | |
| | Min, max | 50.0, 89.0 | | 50.0, 86.0 | | 55.0, 84.0 | | 52.0, 87.0 | |
| Age group, n (%) | <65 years | 55 | (23.1) | 11 | (21.2) | 9 | (18.8) | 20 | (22.5) |
| | ≥65 to <80 years | 144 | (60.5) | 35 | (67.3) | 35 | (72.9) | 60 | (67.4) |
| | ≥80 years | 39 | (16.4) | 6 | (11.5) | 4 | (8.3) | 9 | (10.1) |
| Sex, n (%) | Male | 101 | (42.4) | 26 | (50.0) | 24 | (50.0) | 41 | (46.1) |
| | Female | 137 | (57.6) | 26 | (50.0) | 24 | (50.0) | 48 | (53.9) |
| Ethnicity, n (%) | Hispanic or | 9 | (3.8) | 4 | (7.7) | 1 | (2.1) | 3 | (3.4) |
| | Latino | | | | | | | | |
| | Not Hispanic or | 229 | (96.2) | 48 | (92.3) | 47 | (97.9) | 86 | (96.6) |
| | Latino | | | | | | | | |
| Race, n (%) | White | 216 | (90.8) | 48 | (92.3) | 46 | (95.8) | 7 | (82.0) |
| | Black or African | 5 | (2.1) | 2 | (3.8) | 1 | (2.1) | 4 | (4.5) |
| | American | | | | | | | | |
| | Chinese | 1 | (<1.0) | 0 | | 0 | | 0 | |
| | Japanese | 10 | (4.2) | 1 | (1.9) | 0 | | 6 | (6.7) |
| | Other Asian | 5 | (2.1) | 1 | (1.9) | 1 | (2.1) | 3 | (3.4) |
| | Other | 1 | (<1.0) | 0 | | 0 | | 3 | (3.4) |
| Region, n (%) | North America | 195 | (81.9) | 47 | (90.4) | 41 | (85.4) | 70 | (78.7) |
| | Western Europe | 28 | (11.8) | 4 | (7.7) | 6 | (12.5) | 7 | (7.9) |
| | Asia | 15 | (6.3) | 1 | (1.9) | 1 | (2.1) | 12 | (13.5) |
| CDR-Global, n (%) | 0.5 | 200 | (84.0) | 44 | (84.6) | 40 | (83.3) | 77 | (86.5) |
| | 1 | 38 | (16.0) | 8 | (15.4) | 8 | (16.7) | 12 | (13.5) |
| APOE4 carrier | Carrier | 169 | (71.0) | 38 | (73.1) | 37 | (77.1) | 81 | (91.0) |
| status, n (%) | Heterozygous | 129 | (54.2) | 33 | (63.5) | 26 | (54.2) | 67 | (75.3) |
| | Homozygous | 40 | (16.8) | 5 | (9.6) | 11 | (22.9) | 14 | (15.7) |
| | Non-carrier | 69 | (29.0) | 14 | (26.9) | 11 | (22.9) | 8 | (9.0) |
| Disease stage, | MCI due to AD | 154 | (64.7) | 34 | (65.4) | 33 | (68.8) | 52 | (58.4) |
| n (%) | Mild AD | 84 | (35.3) | 18 | (34.6) | 15 | (31.3) | 37 | (41.6) |
| AChEIs and/or | No | 110 | (46.2) | 24 | (46.2) | 23 | (47.9) | 33 | (37.1) |
| memantine at | | | | | | | | | |
| Baseline, n (%) | Yes | 128 | (53.8) | 28 | (53.8) | 25 | (52.1) | 56 | (62.9) |
| Number of years of | n | 237 | | 52 | | 48 | | 89 | |
| disease since | Mean (SD) | 2.38 | (1.659) | 2.27 | (1.705) | 2.08 | (1.235) | 2.16 | (1.242) |
| diagnosis | Median | 2.00 | | 2.00 | | 2.00 | | 2.00 | |
| | Min, max | 1.0, 11.0 | | 1.0, 7.0 | | 1.0, 6.0 | | 1.0, 6.0 | |
| Age at diagnosis | n | 237 | | 52 | | 48 | | 89 | |
| (years) | Mean (SD) | 70.32 | (8.740) | 69.75 | (8.364) | 69.94 | (7.575) | 70.09 | (7.442) |
| | Median | 70.00 | | 70.00 | | 71.00 | | 71.00 | |
| | Min, max | 50.0, 90.0 | | 49.0, 86.0 | | 54.0, 84.0 | | 52.0, 87.0 | |
| Age at onset of | n | 238 | | 52 | | 48 | | 89 | |
| symptoms (years) | Mean (SD) | 68.00 | (8.880) | 67.35 | (8.220) | 67.13 | (7.601) | 67.93 | (7.513) |
| | Median | 68.00 | | 68.00 | | 67.50 | | 69.00 | |
| | Min, max | 46.0, 88.0 | | 47.0, 83.0 | | 51.0, 82.0 | | 50.0, 87.0 | |
| | | Lecanemab | | | | | | | |
| | | 10 mg/kg | | 10 mg/kg | | | | Combined | |
| | | Monthly | | Biweekly | | Total | | Total | |
| Category | | (N = 246) | | (N = 152) | | (N = 587) | | (N = 825) | |
| Age (year)^{a} | n | 246 | | 152 | | 587 | | 825 | |
| | Mean (SD) | 71.26 | (7.455) | 72.64 | (8.777) | 71.39 | (7.907) | 71.31 | (8.198) |
| | Median | 71.00 | | 73.00 | | 72.00 | | 72.00 | |
| | Min, max | 53.0, 90.0 | | 51.0, 88.0 | | 50.0, 90.0 | | 50.0, 90.0 | |
| Age group, n (%) | <65 years | 44 | (17.9) | 27 | (17.8) | 111 | (18.9) | 166 | (20.1) |
| | ≥65 to <80 years | 168 | (68.3) | 94 | (61.8) | 392 | (66.8) | 536 | (65.0) |
| | ≥80 years | 34 | (13.8) | 31 | (20.4) | 84 | (14.3) | 123 | (14.9) |
| Sex, n (%) | Male | 136 | (55.3) | 88 | (57.9) | 315 | (53.7) | 416 | (50.4) |
| | Female | 110 | (44.7) | 64 | (42.1) | 272 | (46.3) | 409 | (49.6) |
| Ethnicity, n (%) | Hispanic or | 9 | (3.7) | 9 | (5.9) | 26 | (4.4) | 35 | (4.2) |
| | Latino | | | | | | | | |
| | Not Hispanic or | 237 | (96.3) | 143 | (94.1) | 561 | (95.6) | 790 | (95.8) |
| | Latino | | | | | | | | |
| Race, n (%) | White | 222 | (90.2) | 141 | (92.8) | 530 | (90.3) | 746 | (90.4) |
| | Black or African | 4 | (1.6) | 4 | (2.6) | 15 | (2.6) | 20 | (2.4) |
| | American | | | | | | | | |
| | Chinese | 0 | | 0 | | 0 | | 1 | (<1.0) |
| | Japanese | 12 | (4.9) | 5 | (3.3) | 24 | (4.1) | 34 | (4.1) |
| | Other Asian | 5 | (2.0) | 2 | (1.3) | 12 | (2.0) | 17 | (2.1) |
| | Other | 3 | (1.2) | 0 | | 6 | (1.0) | 7 | (<1.0) |
| Region, n (%) | North America | 215 | (87.4) | 135 | (88.8) | 508 | (86.5) | 703 | (85.2) |
| | Western Europe | 15 | (6.1) | 10 | (6.6) | 42 | (7.2) | 70 | (8.5) |
| | Asia | 16 | (6.5) | 7 | (4.6) | 37 | (6.3) | 52 | (6.3) |
| CDR-Global, n (%) | 0.5 | 210 | (85.4) | 133 | (87.5) | 504 | (85.9) | 704 | (85.3) |
| | 1 | 13 | (14.6) | 19 | (12.5) | 83 | (14.1) | 121 | (14.7) |
| APOE4 carrier | Carrier | 218 | (88.6) | 46 | (30.3) | 420 | (71.6) | 589 | (71.4) |
| status, n (%) | Heterozygous | 160 | (65.0) | 38 | (25.0) | 324 | (55.2) | 453 | (54.9) |
| | Homozygous | 58 | (23.6) | 8 | (5.3) | 96 | (16.4) | 136 | (16.5) |
| | Non-carrier | 28 | (11.4) | 106 | (69.7) | 167 | (28.4) | 236 | (28.6) |
| Disease stage, | MCI due to AD | 166 | (67.5) | 90 | (59.2) | 375 | (63.9) | 529 | (64.1) |
| n (%) | Mild AD | 80 | (32.5) | 62 | (40.8) | 212 | (36.1) | 296 | (35.9) |
| AChEIs and/or | No | 115 | (46.7) | 73 | (48.0) | 268 | (45.7) | 378 | (45.8) |
| memantine at | | | | | | | | | |
| Baseline, n (%) | Yes | 131 | (53.3) | 79 | (52.0) | 319 | (54.3) | 447 | (54.2) |
| Number of years of | n | 245 | | 152 | | 586 | | 823 | |
| disease since | Mean (SD) | 2.20 | (1.551) | 2.22 | (1.491) | 2.19 | (1.479) | 2.25 | (1.534) |
| diagnosis | Median | 2.00 | | 2.00 | | 2.00 | | 2.00 | |
| | Min, max | 1.0, 12.0 | | 1.0, 9.0 | | 1.0, 12.0 | | 1.0, 12.0 | |
| Age at diagnosis | n | 245 | | 152 | | 586 | | 823 | |
| (years) | Mean (SD) | 70.71 | (7.526) | 72.03 | (8.855) | 70.81 | (7.971) | 70.67 | (8.197) |
| | Median | 71.00 | | 73.00 | | 71.00 | | 71.00 | |
| | Min, max | 52.0, 90.0 | | 51.0, 89.0 | | 49.0, 90.0 | | 49.0, 90.0 | |
| Age at onset of | n | 246 | | 152 | | 587 | | 825 | |
| symptoms (years) | Mean (SD) | 68.48 | (7.815) | 69.95 | (9.057) | 68.57 | (8.156) | 68.40 | (8.370) |

| | | | | |
|---|---|---|---|---|
| Median | 69.00 | 71.00 | 69.00 | 69.00 |
| Min, max | 45.0, 89.0 | 47.0, 87.0 | 45.0, 89.0 | 45.0, 89.0 |

TABLE 7
Summary of the patient demographics for patients in
the OLE Phase used in the full dataset analysis

| | Lecanemab | |
|---|---|---|
| | 10 mg/kg biweekly | |
| | (N = 180) | |
| Category | n (%) | |
| Age (year)^{a} | | |
| N | 180 | |
| Mean (SD) | 74.0 | (7.69) |
| Median | 74.0 | |
| Min, Max | 52, 87 | |
| Sex, n (%) | | |
| Male | 93 | (51.7) |
| Female | 87 | (48.3) |
| Race, n (%) | | |
| White | 148 | (82.2) |
| Black or African American | 2 | (1.1) |
| Asian | 30 | (16.7) |
| Japanese | 21 | (11.7) |
| Chinese | 1 | (0.6) |
| South Korean | 8 | (4.4) |
| Ethnicity, n (%) | | |
| Hispanic or Latino | 1 | (0.6) |
| Not Hispanic or Latino | 179 | (99.4) |
| Region, n (%) | | |
| North America | 139 | (77.2) |
| Europe | 12 | (6.7) |
| Asia Pacific | 29 | (16.1) |
| APOE4 Status, n (%) | | |
| Positive | 125 | (69.4) |
| Heterozygous | 97 | (53.9) |
| Homozygous | 28 | (15.6) |
| Negative | 55 | (30.6) |
| Concurrent approved symptomatic AD | | |
| treatment at OLE Baseline, n (%) | | |
| Yes | 122 | (67.8) |
| No | 58 | (32.2) |
| Clinical Subgroup at Core Baseline, n (%) | | |
| MCI due to AD | 110 | (61.1) |
| Mild AD dementia | 70 | (38.9) |

TABLE 8
Summary of biomarker results from Study 201 Core.

| | BAN2401 | | |
|---|---|---|---|
| | 10 mg/kg | | |
| Biomarker Endpoints | biweekly | | Placebo |
| Amyloid Beta PET Composite SUVR | N = 44 | | N = 98 |
| Mean baseline | 1.373 | | 1.402 |
| Adjusted mean change from baseline at Week 53 | −0.266 | | −0.009 |
| Difference from placebo | −0.257 | (p < 0.001) | |
| Adjusted mean change from baseline at Week 79 | −0.306 | | 0.004 |
| Difference from placebo | −0.310 | (p < 0.001) | |
| Amyloid Beta PET Centiloid | N = 44 | | N = 98 |
| Mean baseline | 78.02 | | 84.75 |
| Adjusted mean change from baseline at Week 53 | −62.827 | | −2.154 |
| Difference from placebo | −60.673 | (p < 0.001) | |
| Adjusted mean change from baseline at Week 79 | −72.495 | | 1.004 |
| Difference from placebo | −73.499 | (p < 0.001) | |
| Conversion to Amyloid Negative (visual read) | N = 44 | | N = 99 |
| % Amyloid negative at Week 53 | 65.1% | | 11.5% |
| % Amyloid negative at Week 79 | 81.1% | | 21.6% |
| Plasma Aβ42/40 | N = 43 | | N = 88 |
| Mean baseline | 0.0842 | | 0.0855 |
| Adjusted mean change from baseline at Week 53 | 0.0049 | | 0.0000 |
| Difference from placebo | 0.0048 | (p = 0.0029) | |
| Adjusted mean change from baseline at Week 79 | 0.0075 | | 0.0021 |
| Difference from placebo | 0.0054 | (p = 0.0036) | |
| Plasma p-tau181 (pg/mL) | N = 84 | | N = 179 |
| Mean baseline | 4.6474 | | 4.435 |
| Adjusted mean change from baseline at Week 53 | −1.2054 | | 0.0664 |
| Difference from placebo | −1.2718 | (p < 0.0001) | |
| Adjusted mean change from baseline at Week 79 | −1.1127 | | 0.0832 |
| Difference from placebo | −1.1960 | (p < 0.0001) | |
| CSF p-tau181 (pg/mL) | N = 12 | | N = 24 |
| Adjusted mean change from baseline at Week 53 | −9.732 | | 3.258 |
| Difference from placebo | −12.990 | (p = 0.078) | |
| Adjusted mean change from baseline at Week 79 | −10.880 | | 1.436 |
| Difference from placebo | −12.315 | (p = 0.044) | |

N is the number of patients with baseline value.

TABLE 9
Summary of biomarker results of OLE Phase

| | BAN2401 | |
|---|---|---|
| Biomarker Endpoints | 10 mg/kg biweekly* | |
| Amyloid Beta PET Composite SUVR | N = 22 | |
| Mean OLE baseline | 1.370 | |
| Adjusted mean change from baseline at Week 13 | −0.088 | (p < 0.001) |
| Adjusted mean change from baseline at Week 27 | −0.157 | (p < 0.001) |
| Adjusted mean change from baseline at Week 53 | −0.232 | (p < 0.001) |
| Amyloid Beta PET Centiloid | N = 22 | |
| Mean OLE baseline | 77.20 | |
| Adjusted mean change from baseline at Week 13 | −18.347 | (p = 0.022) |
| Adjusted mean change from baseline at Week 27 | −40.921 | (p < 0.001) |
| Adjusted mean change from baseline at Week 53 | −56.328 | (p < 0.001) |

| | |
|---|---|
| Conversion to Amyloid Negative (visual read) | N = 17 |
| % Amyloid negative at Week 13 | 42.9% |
| % Amyloid negative at Week 27 | 75.0% |
| % Amyloid negative at Week 53 | 83.3% |
| Plasma Aβ42/40 | N = 32 |
| Mean OLE baseline | 0.0857 |
| Mean change from baseline at Week 13 | 0.0028 |
| Mean change from baseline at Week 27 | 0.0044 |
| Mean change from baseline at Week 53 | 0.0097 |
| Plasma p-tau181 (pg/mL) | N = 27 |
| Mean OLE baseline | 5.272 |
| Mean change from baseline at Week 13 | 0.089 |
| Mean change from baseline at Week 27 | −0.921 |
| Mean change from baseline at Week 53 | −1.596 |

*Patients who were randomized to placebo in the double-blind, placebo-controlled period of Study 201 Core and received 10 mg/kg biweekly in the Open-Label Extension

TABLE 10
Summary results of clinical outcomes in Study 201 Core.

| | BAN2401 | | |
|---|---|---|---|
| Clinical Endpoints | 10 mg/kg biweekly | | Placebo |
| ADCOMS | N = 152 | | N = 238 |
| Mean baseline | 0.373 | | 0.370 |
| Adjusted mean change from baseline at Week 53 | 0.085 | | 0.131 |
| Difference from placebo | −0.046 | (p = 0.027) | |
| Adjusted mean change from baseline at Week 79 | 0.136 | | 0.193 |
| Difference from placebo | −0.057 | (p = 0.034) | |
| CDR-SB | N = 152 | | N = 238 |
| Mean baseline | 2.97 | | 2.89 |
| Adjusted mean change from baseline at Week 53 | 0.568 | | 0.911 |
| Difference from placebo | −0.344 | (p = 0.077) | |
| Adjusted mean change from baseline at Week 79 | 1.102 | | 1.499 |
| Difference from placebo | −0.396 | (p = 0.125) | |
| ADAS-Cog 14 | N = 152 | | N = 237 |
| Mean baseline | 22.06 | | 22.56 |
| Adjusted mean change from baseline at Week 53 | 1.481 | | 2.842 |
| Difference from placebo | −1.361 | (p = 0.073) | |
| Adjusted mean change from baseline at Week 79 | 2.588 | | 4.902 |
| Difference from placebo | −2.313 | (p = 0.017) | |

N is the number of patients with baseline value.
All p-values are nominal.

### Mean Change in Aβ42/40 Ratio by Core Treatment Group and Visit—Core/Open Label Extension (OLE)

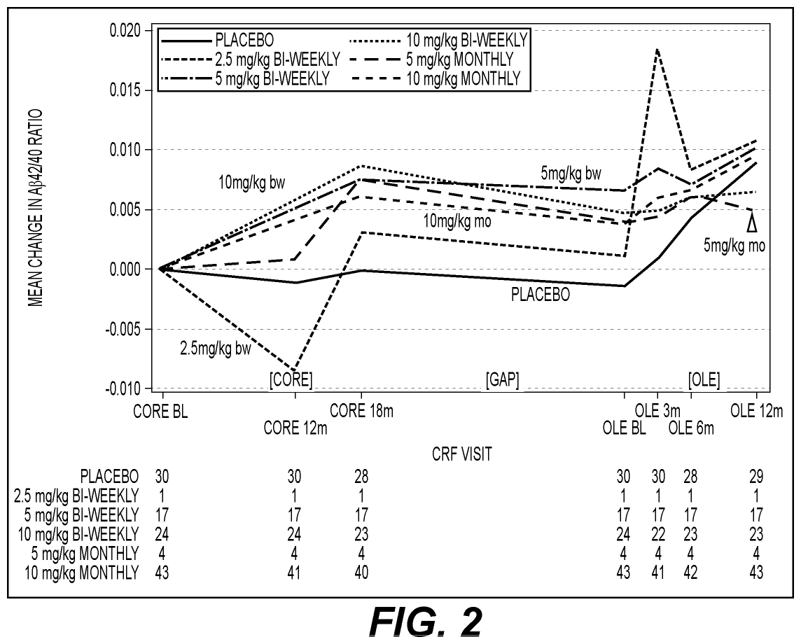

Analysis Population: Subjects with Plasma Aβ42/40 Ratio at Core Baseline (BL) and at Least One Postbaseline Visit (FIG. 2)

[Core] Plasma Aβ42/40 ratio increased in Core 10 mg/kg biweekly (Core 10bw) and Core 10 mg/kg monthly (Core 10mo) during Core Study, with a slight decrease in the Core Placebo group, indicating expected subtle accumulation of Aβ plaques. Changes for Placebo and 10 mg/kg biweekly and 10 mg/kg monthly lecanemab were dose-dependent.

[Gap] There was an intervening gap period off-treatment between the core study and the OLE Phase. Plasma Aβ42/40 ratio slightly decreased in all three groups during Gap period.

[OLE] During the OLE extension, all cohorts (including the placebo cohort form the Core study) received 10 mg/kg biweekly. Plasma Aβ42/40 ratio increased in all three groups during OLE. The mean change in plasma Aβ42/40 ratio was dependent on OLE baseline amyloid levels. All subsequent analyses were focus on the Placebo, 10 mg/kg biweekly and 10 mg/kg monthly.

Similar trends were seen in the analysis of additional patients. Lecanemab demonstrated a dose-dependent and time-dependent increase in plasma Aβ42/40 across all doses versus placebo, with statistical significance seen at 12 and 18 months in the lecanemab 10 mg/kg monthly (P≤0.0388) and lecanemab 10 mg/kg biweekly (P≤0.0036) dose groups. The least squares (LS) mean changes from Baseline in plasma Aβ42/40 ratio at 18 months are presented in FIG. 31.

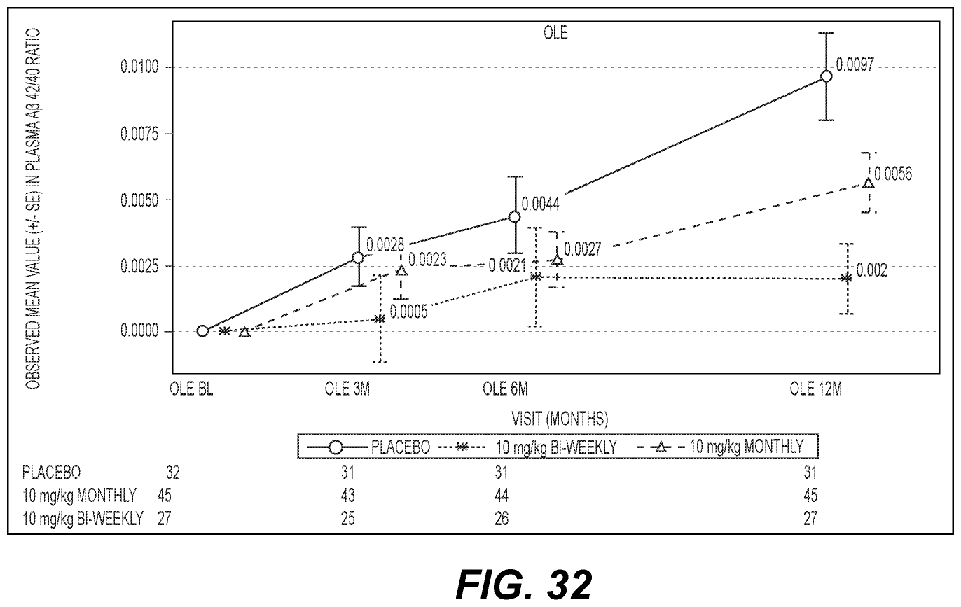

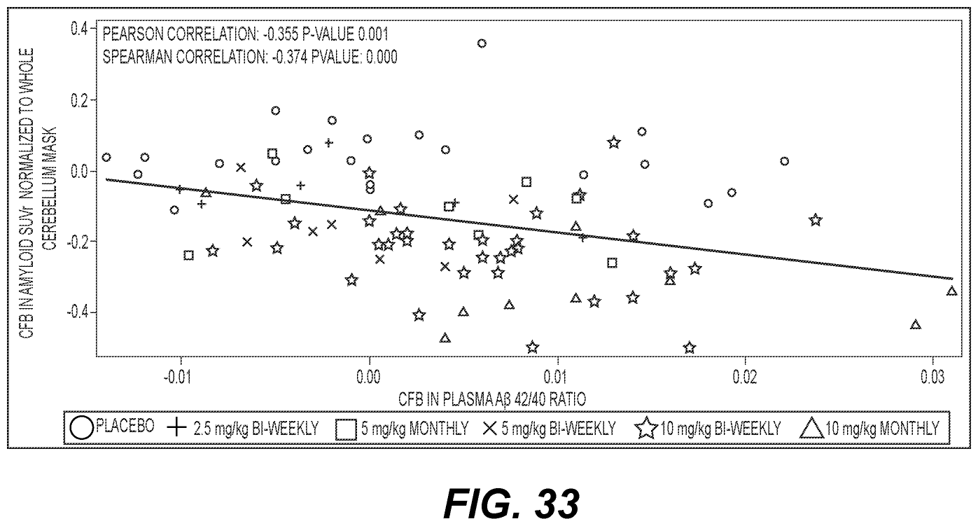

In the OLE Phase, both newly-treated Core Study placebo subjects and re-treated lecanemab 10 mg/kg biweekly and lecanemab 10 mg/kg monthly subjects showed an increase in plasma Aβ42/40 ratio following treatment with lecanemab 10 mg/kg biweekly (FIG. 32), which, without being bound by theory, is likely associated with lecanemab clearing newly generated amyloid including protofibrils even after amyloid plaque removal. The largest increase in Aβ42/40 ratio is seen in the newly treated Core Placebo group, the group which had the largest PET SUVr reduction from OLE Baseline, with a statistically significant reduction P<0.001 seen as early as 3 months (Week 13 Visit, amyloid reduction of 0.09 SUVR) and amyloid reduction of 0.16 (p<0.001) SUVR at Week 27, which was maintained through to the 24 month visit in the OLE.

### Mean Change in Aβ42/40 Ratio and PET SUVR by Core Treatment Group—Core/OLE

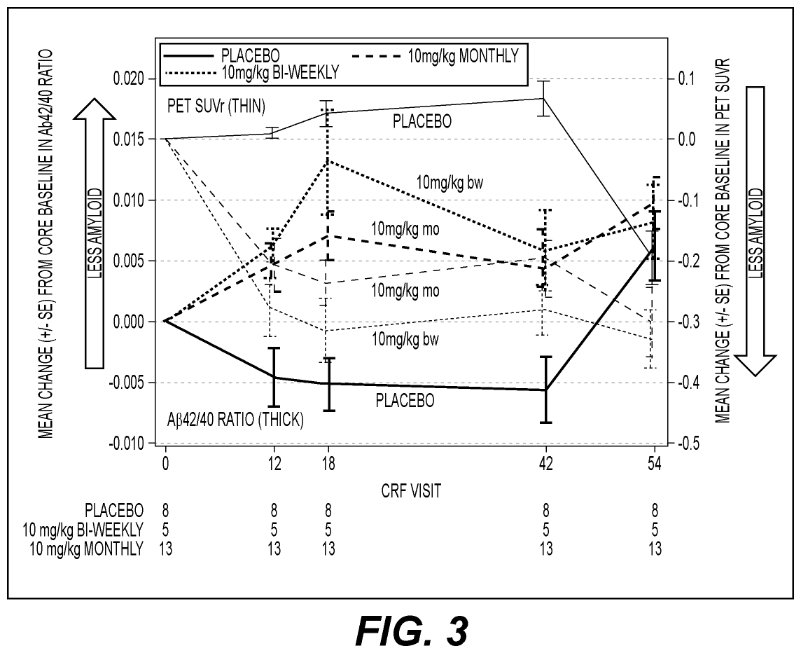

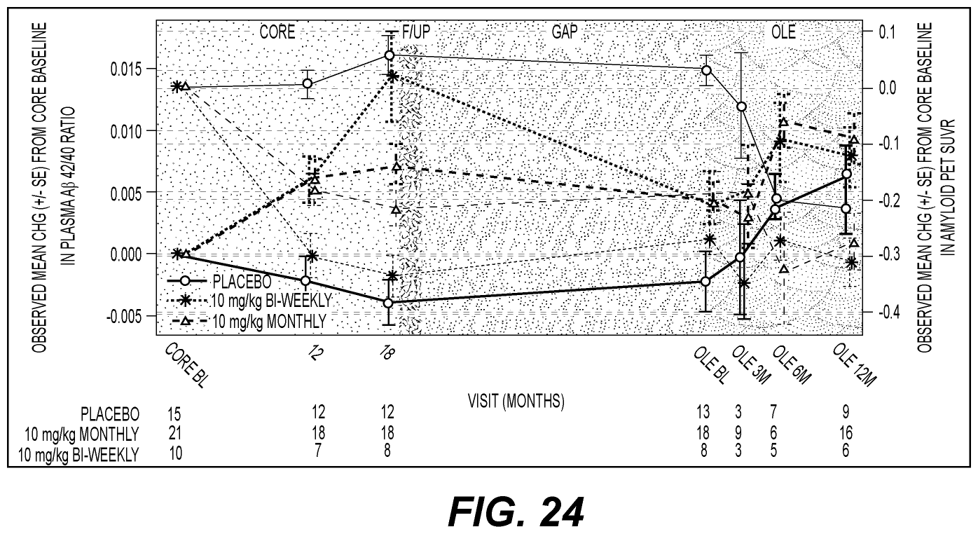

Analysis population: subjects with both plasma ratio and PET SUVR (by Florbetapir) at Core Baseline, Core 18 months, OLE BL, and OLE 12 months (FIG. 3). Similar trends were seen with the analysis of additional patients, as provided in FIG. 24.

Longitudinal changes in amyloid assessed by PET SUVr were mirrored by (inverse) changes in Aβ42/40 ratio for all three Core treatment groups (Placebo, 10 mo, 10 bw) across Core, Gap, and longitudinal OLE.

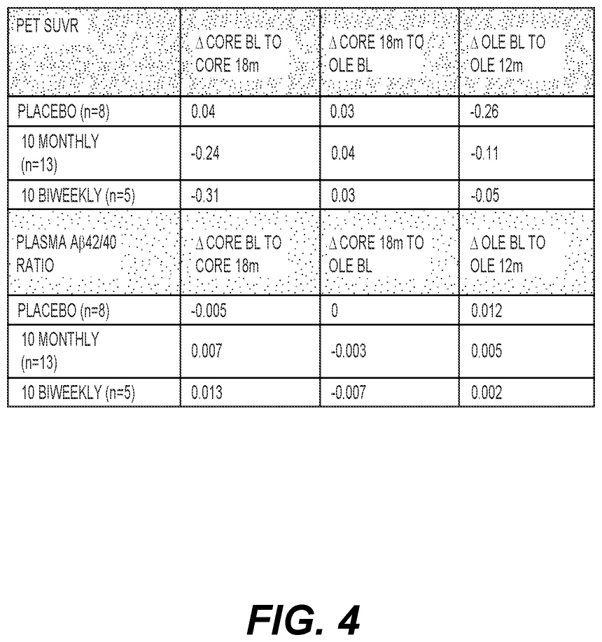

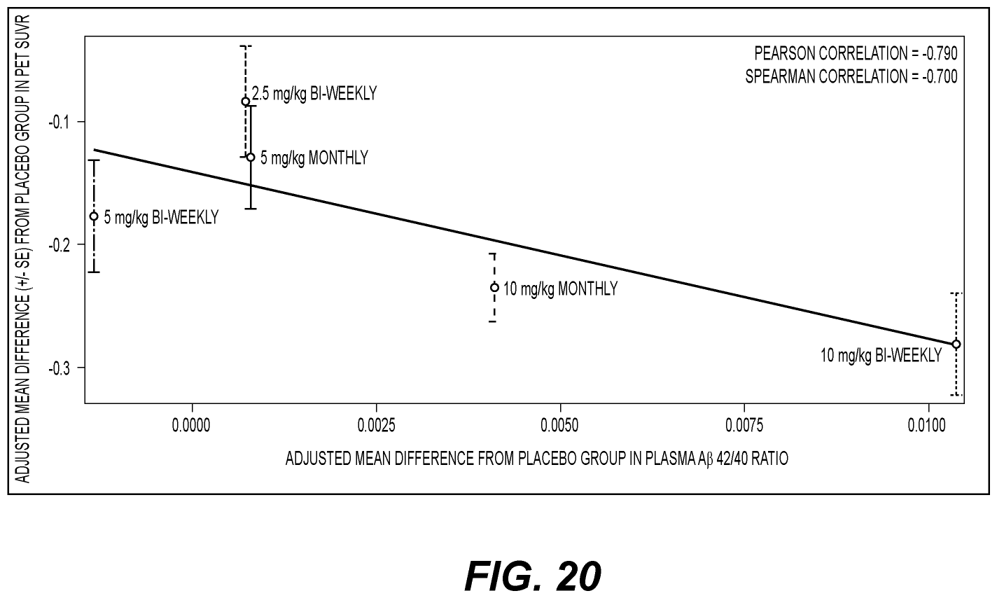

FIG. 4 is a summary table of change in PET SUVr and Plasma Aβ42/40 ratio during Core and OLE phases. FIG. 20 provides further analysis with additional patient data from the Core phase comparing the change from baseline in plasma Aβ42/40 ratio and amyloid PET SUVr values at 18 months (Pearson correlation coefficient=−0.790, P=0.112.) FIG. 32 is a scatter plot of individual patient data from the Core phase comparing the change from baseline in plasma Aβ42/40 ratio and amyloid PET SUVr values at 18 months (Pearson correlation coefficient=−0.355, P=0.001).

Mean Change in Aβ42/40 ratio and PET SUVR by Core Treatment Group—OLE

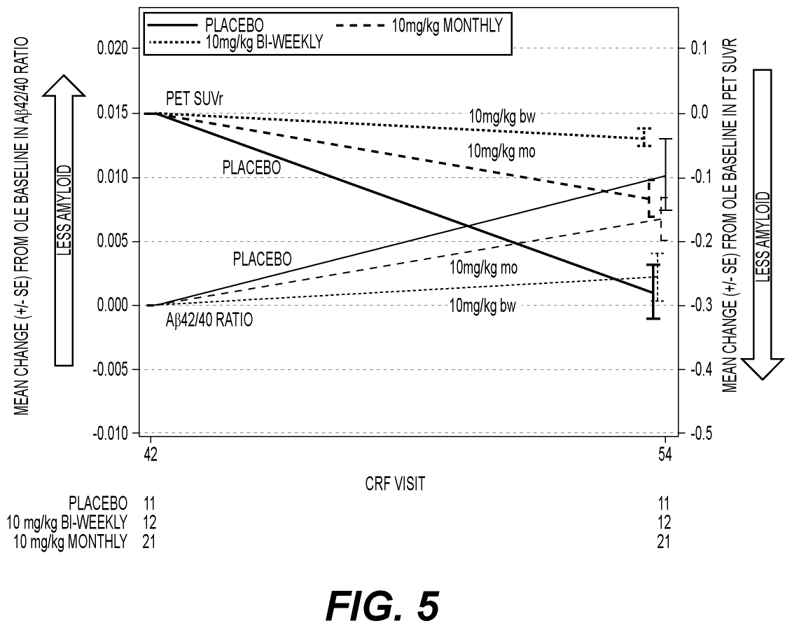

Analysis population: subjects with both plasma ratio and PET SUVR (by Florbetapir) at OLE BL and OLE 12 m (FIG. 5)

Longitudinal changes in amyloid in OLE assessed by PET SUVr were mirrored by (inverse) changes in Aβ42/40 ratio for all three Core treatment groups (Pbo, 10 M, 10 bw)

Changes for both PET SUVr and Aβ42/40 ratio were dependent on Core treatment assignment and corresponding OLE Baseline amyloid levels as measured by PET SUVr

### Scatter Plot of Mean Change in Aβ42/40 Ratio and PET SUVR by Core Treatment Group and Visit—OLE

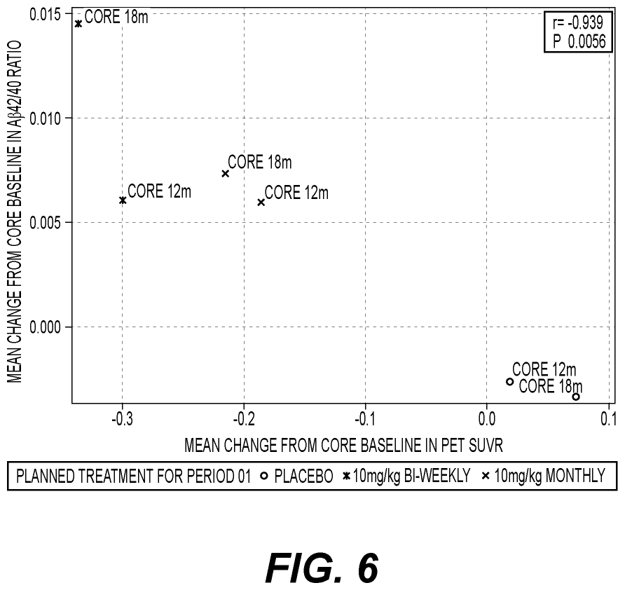

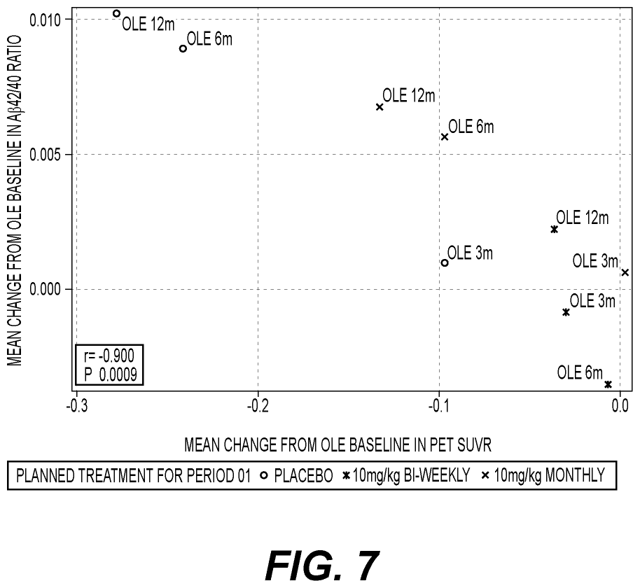

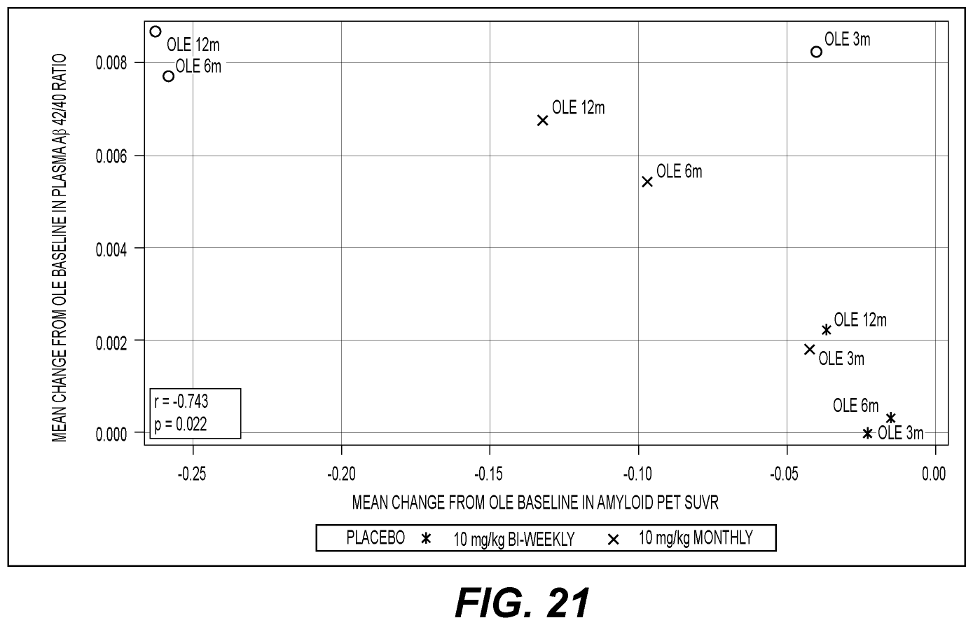

Analysis population: subjects with both plasma ratio and PET SUVR (by Florbetapir) at Core BL, Core 18 m, OLE BL, and OLE 12 m
r=−0.939 (P=0.0056)
   Mean change in ratio was negatively correlated with mean change in PET SUVR (population level) during Core (FIG. 6)
r=−0.900 (P=0.0009)
   Mean change in ratio was negatively correlated with mean change in PET SUVR (population level) during OLE (FIG. 7). Patients that received placebo in Core and then received 10 mg/kg biweekly during the OLE saw improvements in plasma Aβ42/40 ratio and amyloid PET SUVr before 12 months of treatment, e.g., within 3 months or 6 months. A further analysis with additional patient data from the OLE is provided in FIG. 21.

### Scatter Plots Between Aβ42/40 Ratio and PET SUVR—Core

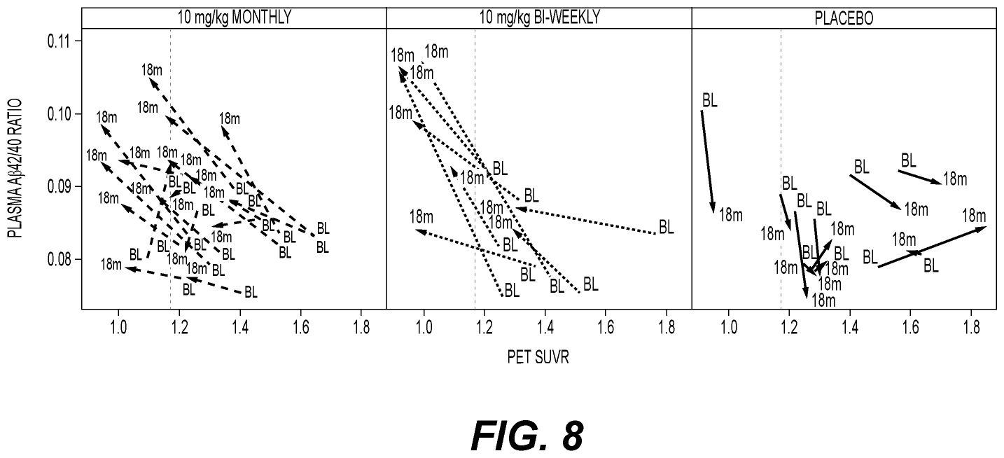

Analysis population: subjects with both plasma ratio and PET SUVR (by Florbetapir) at Core BL and Core 18 m (FIG. 8)
Based on all subjects with plasma ratio and PET SUVR at both Core baseline and Core 18 m
   11 out of 30 subjects in Core Placebo
   17 out of 43 subjects in Core 10mo
   8 out of 24 subjects in Core 10bw
10 bw/10mo showed improvement in both plasma Aβ42/40 ratio and PET SUVR (arrows rising from right to left) at Core
   All 10 mg/kg biweekly subjects show improvement on both plasma Aβ42/40 ratio and PET SUVR

### Scatter Plots Between Aβ42/40 Ratio and PET SUVR—OLE

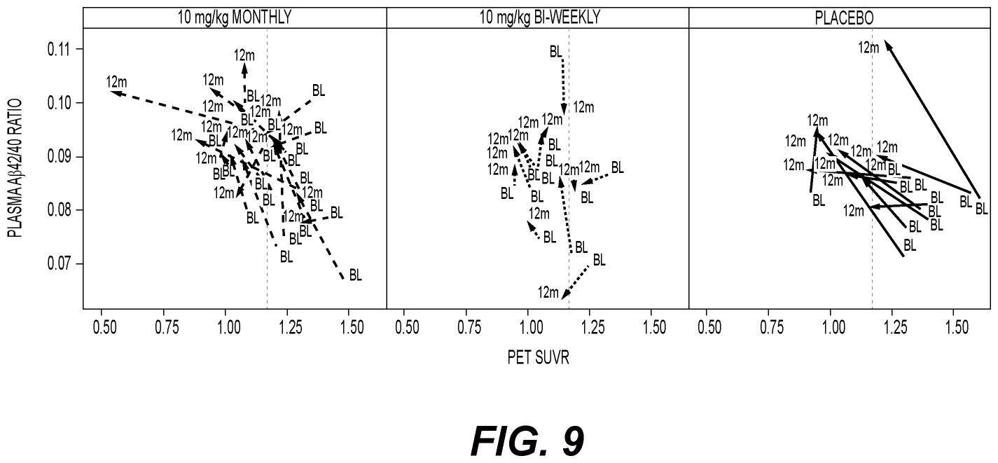

Analysis population: subjects with both plasma ratio and PET SUVR (by Florbetapir) at OLE BL and OLE 12 m (FIG. 9)
Based on all subjects with plasma ratio and PET SUVR at both OLE baseline and OLE 12 m
   11 out of 30 subjects in Core Placebo
   21 out of 43 subjects in Core 10mo
   12 out of 24 subjects in Core 10bw
Core Placebo treated subjects showed improvement in both plasma Aβ42/40 ratio and PET SUVR (arrows rising from right to left) in OLE
Nearly all Core placebo treated subjects showed improvement in both plasma Aβ42/40 ratio and PET SUVR

Plasma Aβ42/40 ratio showed dose-dependent increase in Core over 18 months of treatment. Plasma Aβ42/40 ratio and PET SUVr changes tracked for all doses across Core, GAP, and OLE. Rate of change in brain amyloid reduction (slopes) in OLE for both PET SUVr and Aβ42/40 ratio were dependent on Core treatment assignment and associated brain amyloid levels at OLE Baseline. Mean change in plasma Aβ42/40 ratio was negatively correlated with mean change in PET SUVR at both the group and individual levels. Plasma Aβ42/40 ratio also correlated with clinical endpoints such as CDR-SB, ADAS-Cog14, and ADCOMS. For example, in the Core study CDR-SB showed correlation with plasma Aβ42/40 ratio (r=−0.745, P=−0.0894). These findings suggest potential to use the plasma Aβ42/40 ratio assay to monitor for drug effects in individual subjects/patients who are under treatment with lecanemab. Such monitoring using plasma Aβ42/40 ratio assay may enable evaluating a response to the treatment with lecanemab and/or to guide further treatment regimens which may include dose reduction or increase of lecanemab, changes in dosing interval, discontinuation of the treatment with lecanemab, or switching to other treatment options with or without lecanemab.

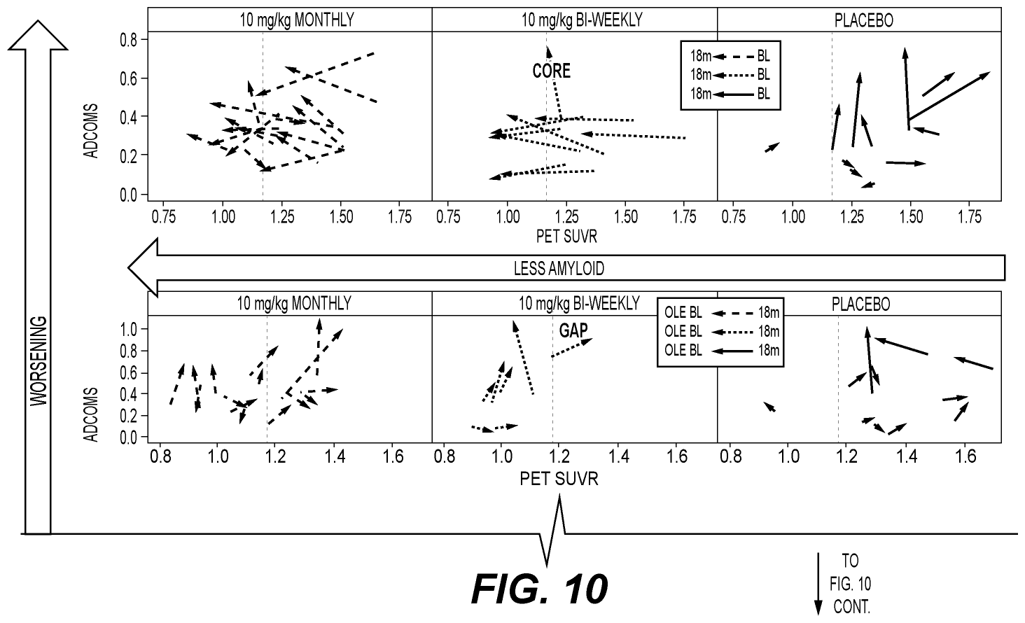

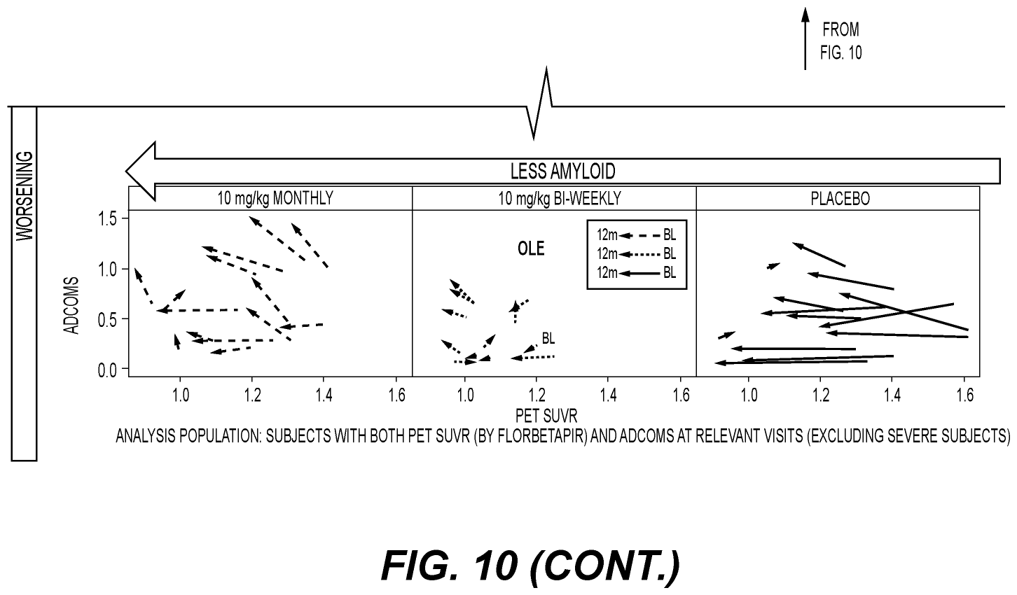

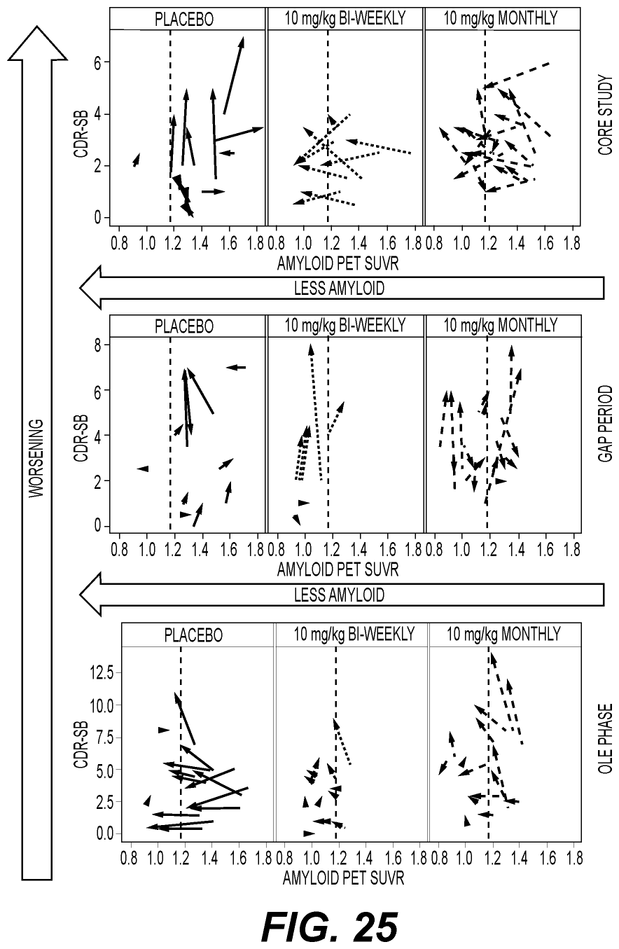

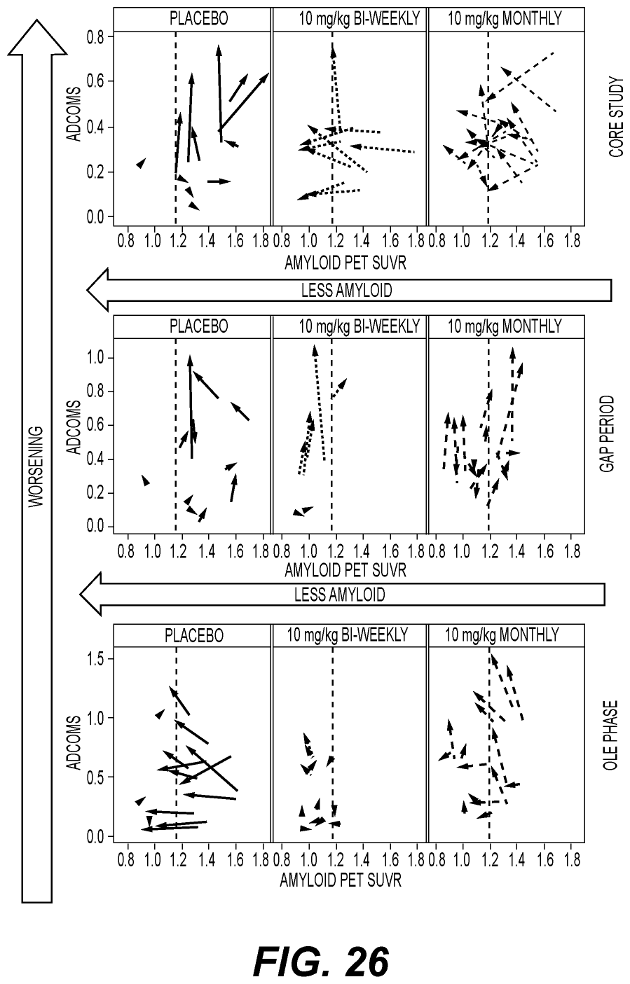

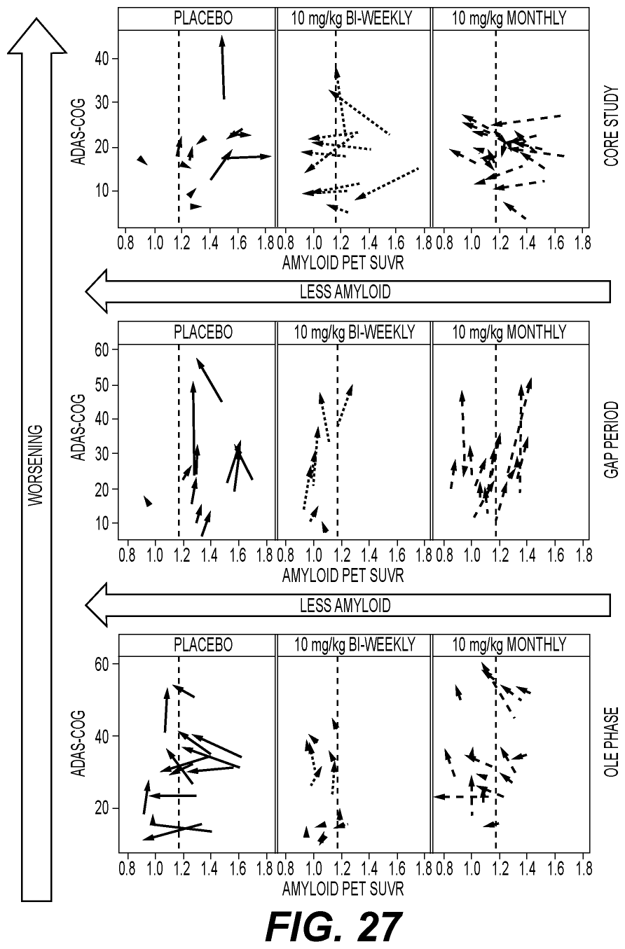

Individual Data Correlation of PET SUVr with ADCOMS During Core, Gap, & OLE
PET SUVr correlates with CDR-SB during Core (r=0.936, p=0.0060). Correlation between them is not significant during Gap (r=0.435, p=0.7137) & OLE (r=−0.195, p=0.7114), though trend is in the same direction for both. Need to interpret results with caution due to small sample size. A further analysis with additional patient data is provided in FIG. 25.
PET SUVr correlates with ADCOMS during Core (r=0.790, p=0.0614). Correlation between them is not significant during Gap (r=0.663, p=0.5385) & OLE (r=−0.154, p=0.7706), though trend is in the same direction for both (FIG. 10). A further analysis with additional patient data is provided in FIG. 26.
PET SUVr correlates with ADAS-Cog during Core (r=0.590, p=0.2177). Correlation between them is not significant during Gap (r=0.958, p=0.1843) & OLE (r=0.118, p=0.8237), though trend is in the same direction for both. A further analysis with additional patient data is provided in FIG. 27.
   Individual Data Correlation of Aβ42/40 Ratio with ADCOMS During Core, Gap, & OLE

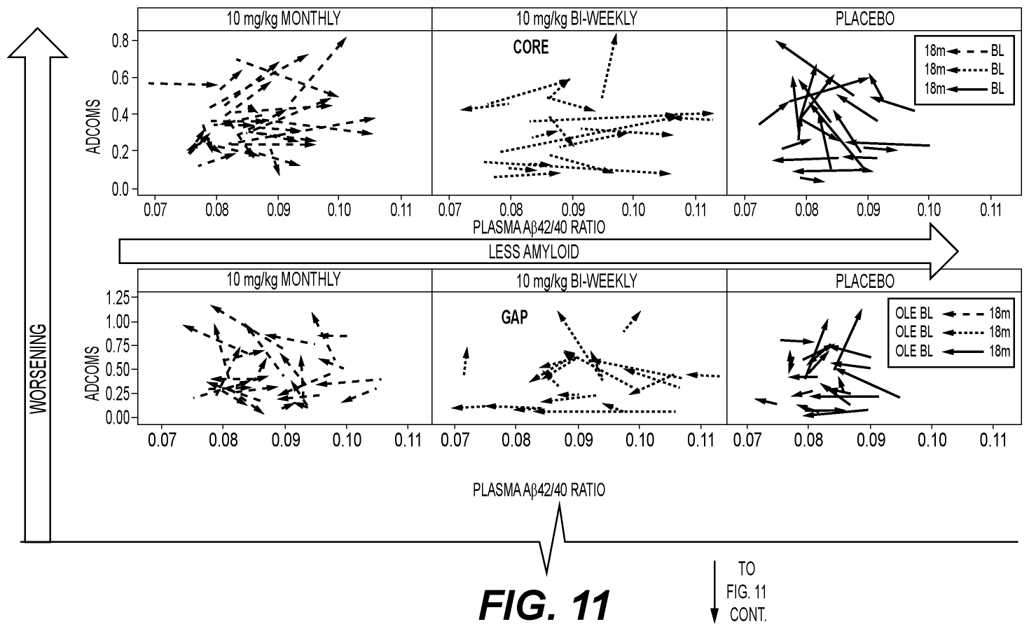

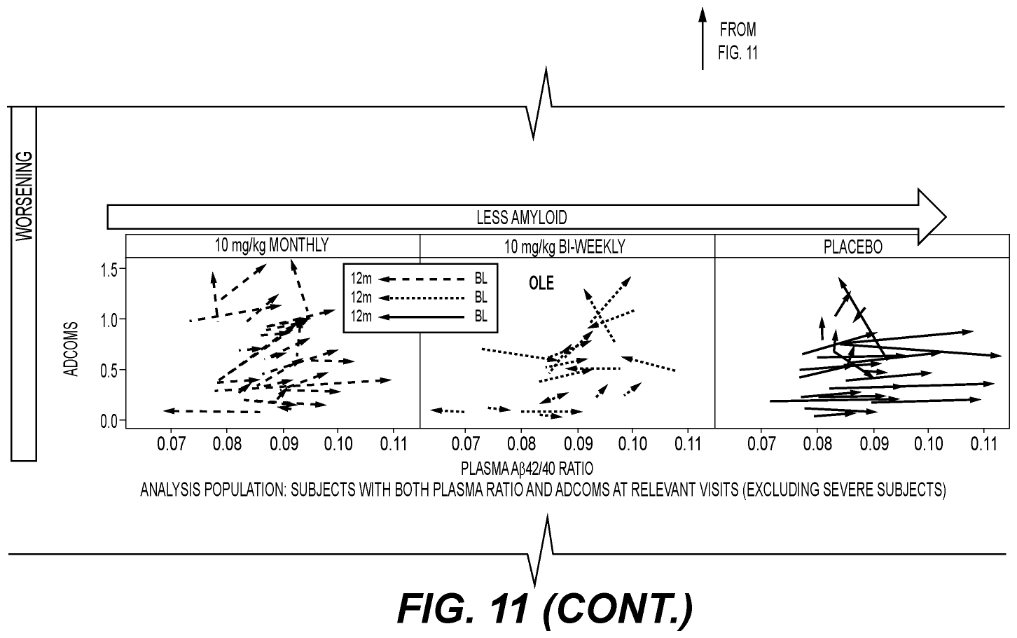

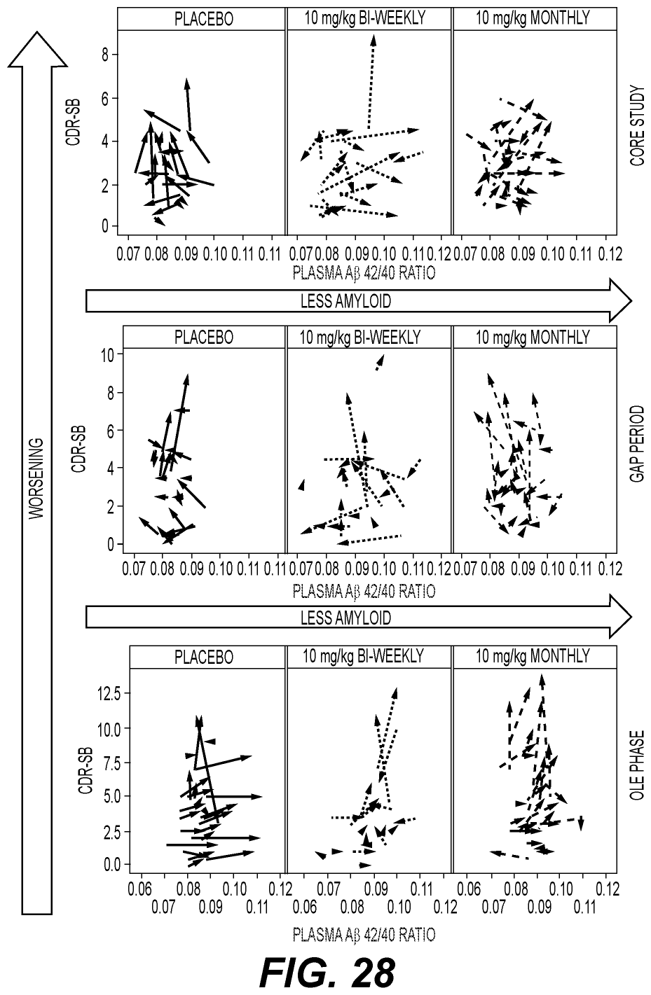

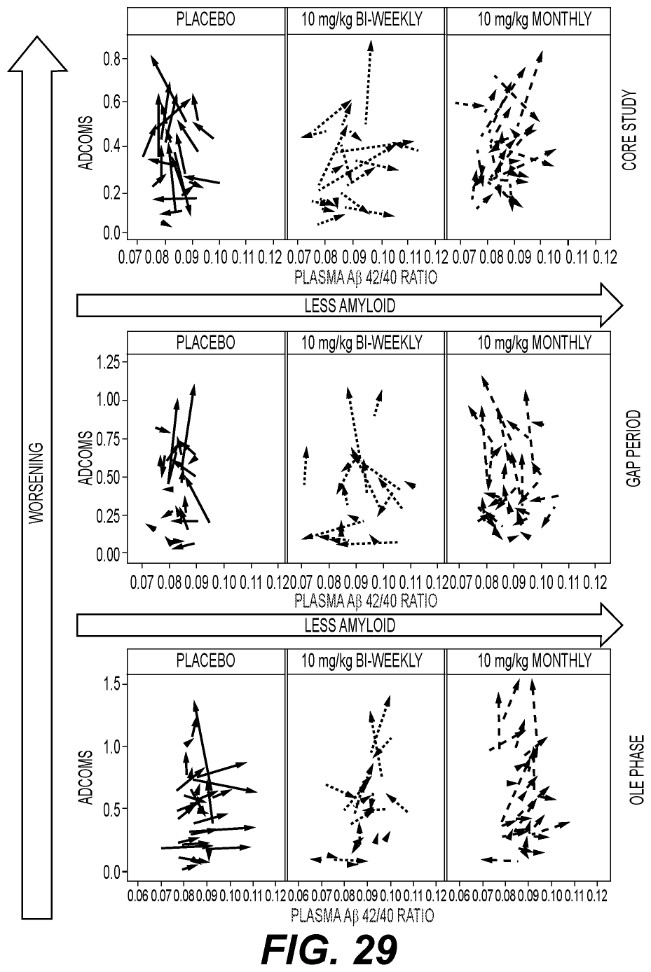

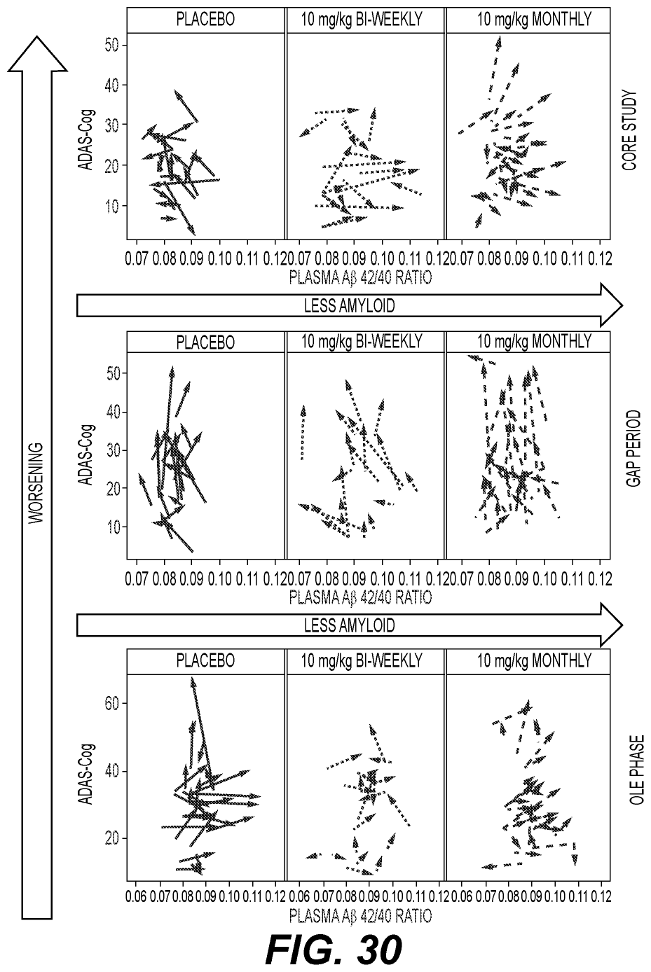

Longitudinal changes in plasma Aβ42/40 ratio inversely correlates with CDR-SB during Core (r=−0.745, p=0.0894). Correlation between them is not significant during Gap (r=−0.190, p=0.8782) & OLE (r=0.174, p=0.7420), though trend is in the same direction for both. A further analysis with additional patient data is provided in FIG. 28.
Longitudinal changes in plasma Aβ42/40 ratio inversely correlates with ADCOMS during Core (r=−0.672, p=0.1435). Correlation between them is not significant during Gap (r=0.054, p=0.9654) & OLE (r=−0.005, p=0.9919), though trend is in the same direction for both (FIG. 11). A further analysis with additional patient data is provided in FIG. 29.
Similar results seen for ADAS-Cog, though not statistically significant. r=−0.482, p=0.3330 in Core, r=0.558, p=0.6232 in Gap & r=0.243, p=0.6432 in OLE. A further analysis with additional patient data is provided in FIG. 30.
   Individual and Population Data Correlation of Aβ42/40 Ratio with ADCOMS, CDR-SB and ADAS-Cog During Core

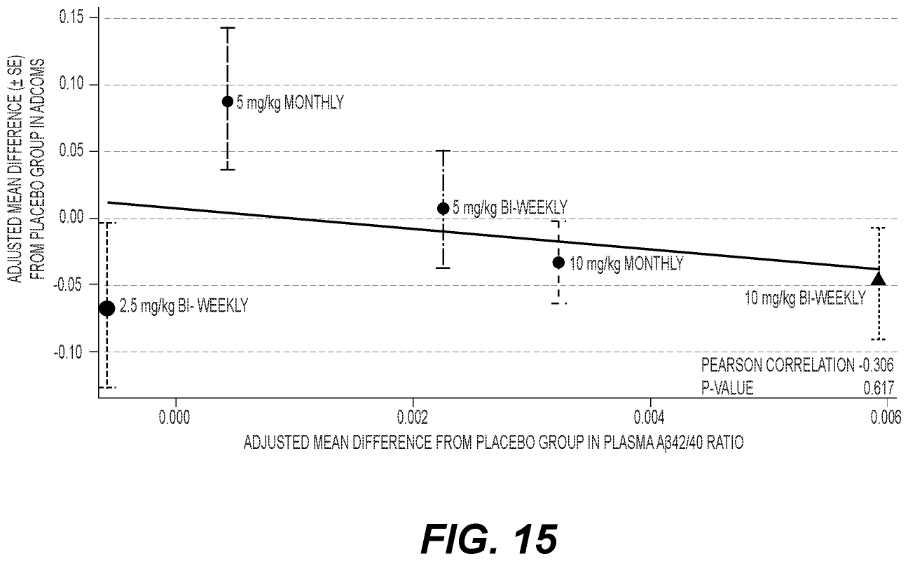

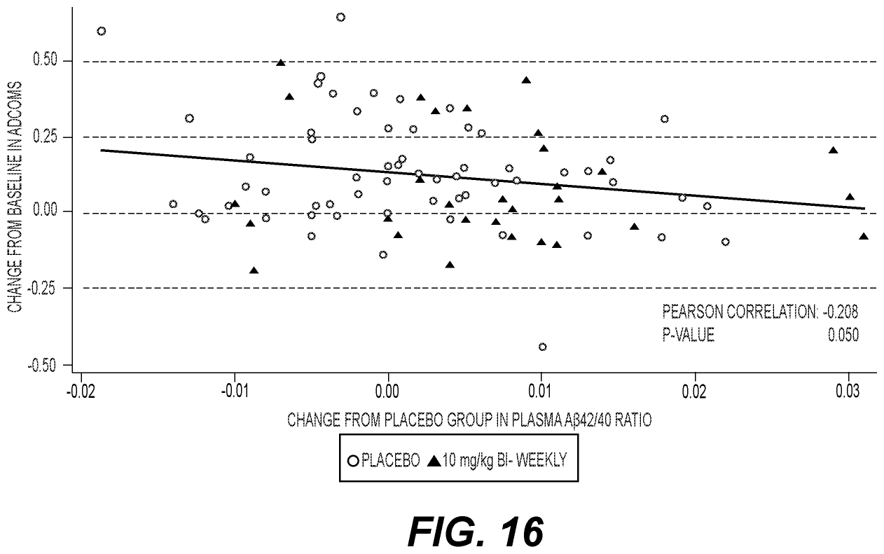

Population longitudinal changes in plasma Aβ42/40 ratio relative to baseline were assessed in subjects and were inversely correlated with ADCOMS during Core, although the correlation between them is not significant (r=−0.306, p=0.617)(FIG. 15). Individual longitudinal changes in plasma Aβ42/40 ratio inversely correlated with ADCOMS during Core (r=−0.208, p=0.050) (FIG. 16).

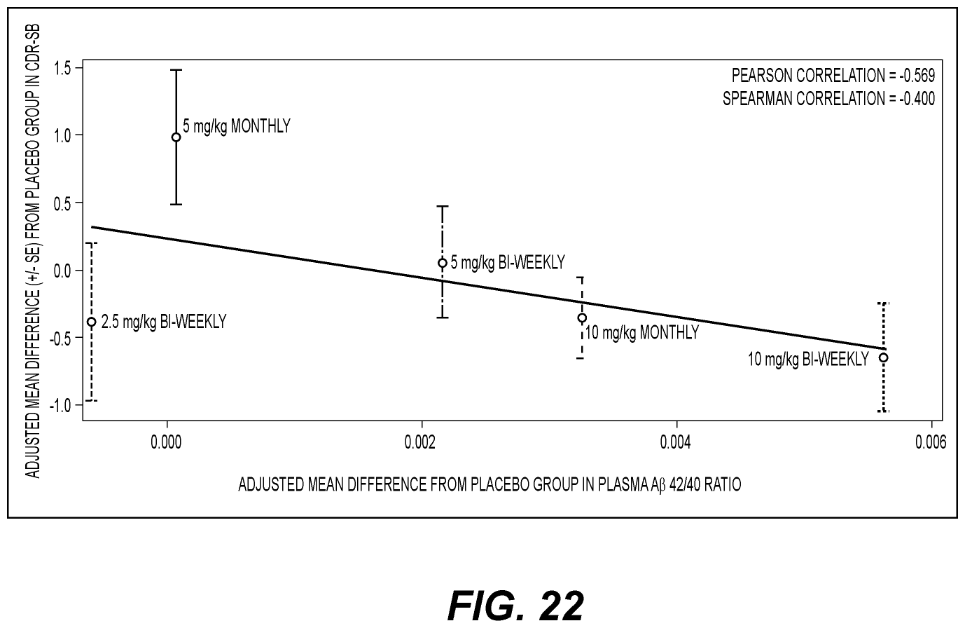

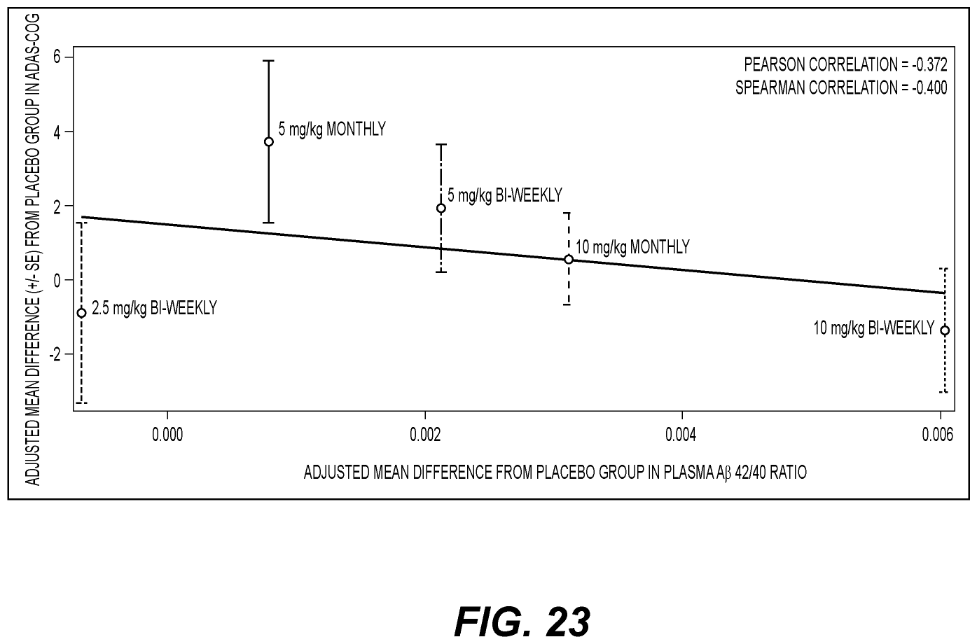

Population longitudinal changes in plasma Aβ42/40 ratio relative to baseline were assessed in subjects and were inversely correlated with CDR-SB during Core (FIG. 22). Population longitudinal changes in plasma Aβ42/40 ratio relative to baseline were assessed in subjects and were also found to be inversely correlated with ADAS-Cog during Core (FIG. 23).

### 2. Treatment of Subjects Prior to Cognitive Impairment

The AHEAD 3-45 Study is testing whether intervention with lecanemab (BAN2401), a humanized IgG1 monoclonal antibody that preferentially targets soluble aggregated Aβ, initiated prior to cognitive impairment, can slow accumulation of tau and prevent cognitive decline. The AHEAD Study consists of two sister trials (Aβ and A45) in cognitively unimpaired (CU) individuals ages 55-80. Similar to the experience in the A4 Study and other secondary prevention trials in preclinical AD, the screening process can be time consuming and costly with thousands of PET scans. Thus, a need exists to accelerate and improve the efficiency of identifying CU individuals beyond PET imaging. Recent advances in blood-based biomarkers suggest that plasma measures can detect evidence of AD pathology with reasonable accuracy, even at the preclinical stage of AD, and thus, may effectively triage individuals most appropriate to undergo screening PET imaging.

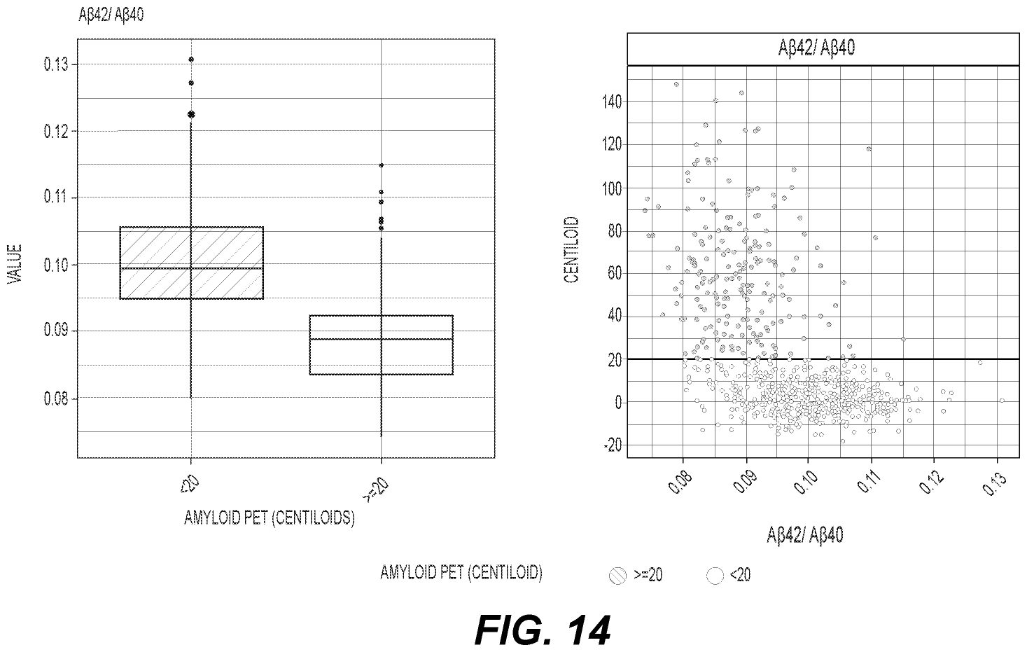

The AHEAD 3-45 consists of two sister trials (Aβ and A45) with specific dosing regimens tailored to baseline brain amyloid levels on screening PET scans: for Aβ intermediate amyloid (approximately 20-40 centiloids) and for A45 elevated amyloid (>40 centiloids). Both trials exist under a single protocol, screening process, and common schedule of assessments. The trials will include plasma screening with C2N Diagnostics' mass spectrometry platform (PrecivityAD™) to quantitate the amyloid beta 42/40 ratio (Aβ 42/40), which has been previously shown to be a reliable predictor of brain amyloid level (FIG. 14). Blood samples will be collected at a brief initial visit and used to determine eligibility to proceed in the screening process to PET imaging. Eligibility to randomize into the Aβ or A45 study will still be based on the screening PET imaging results.

Over 1150 participants are in or have completed the screening process for the AHEAD Study at more than 80 active sites in the US, Japan, UK, Singapore and Australia and 717 have completed screening amyloid PET imaging. Currently, 9.9% of those participants are into the intermediate amyloid range (potentially eligible for Aβ), and 22.4% are in the elevated range (potentially eligible for A45), but these rates vary by age and APOE ε4 carrier status. The first 700 plasma samples are being processed. The initial threshold for eligibility to proceed to PET imaging will be determined based on the AUC results using receiver operating characteristic (ROC) curve analysis from these first 700 participants.

Data from 659 participants was analyzed. Adjusted Aβ42/40 ratio demonstrated very good ability to predict amyloid PET eligibility (AUC of 0.87), suggesting plasma screening has potential to substantially reduce number of PET scans needed to fully enroll Aβ and A45.

The AHEAD 3-45 Study will be the first secondary prevention trial to employ plasma-based biomarkers to accelerate the screening process and potentially substantially decrease the number of screening PET scans. Additional plasma biomarkers and demographic information will be utilized to optimize the screening algorithm.

3. Treatment at 10 mg/kg Biweekly for 18 Months Followed by Treatment Discontinuation or Alternative Dosing Frequency for 24 Months

### Objective: To Explore the Effects of Dosing and Dosing Frequency on Amyloid PET SUVr, and Aβ42/40.

Methods: From the PK model previously developed with PK data from earlier studies, including Core and OLE, Bayesian predictions of PK parameters for each subject were made, using the nonlinear mixed effects model (NONMEM) POSTHOC function. For Bayesian predictions of PK parameters for each subject, a final PK model with the existing dataset was used. These predicted parameters were used to generate individually predicted PK profiles, which were then incorporated into the PK/PD datasets to be used in the subsequent PK/PD analyses.

### PK/PD Model for Plasma Aβ42/40 Ratio (Study 201 Core and OLE)

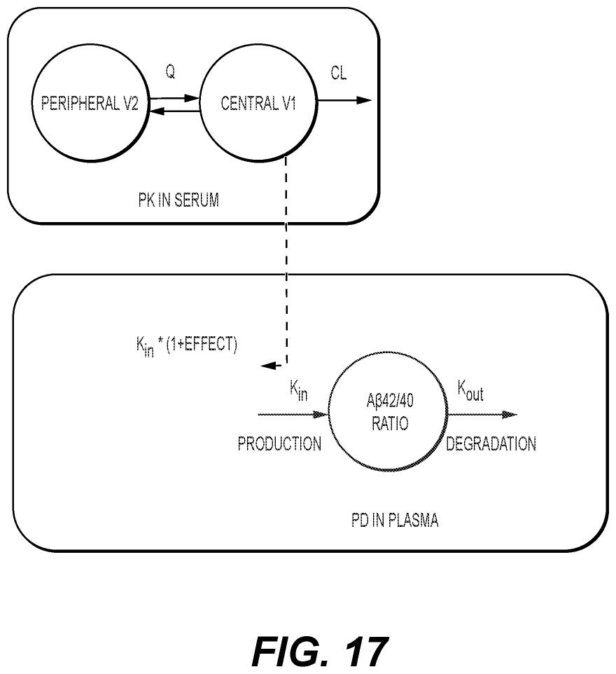

Absolute plasma Aβ42/40 ratio measurements over time (R(t)) were related to model-predicted lecanemab serum concentration at the time of the assessment. A low Aβ42/40 ratio is indicative of elevated amyloid in the brain, therefore treatment aimed toward reducing brain amyloid is expected to increase the plasma Aβ42/40 ratio. The relationship between the lecanemab concentration and the Aβ42/40 ratio change time course was described by an indirect response model with the lecanemab concentration increasing the plasma Aβ42/40 ratio (Kᵢₙ), as indicated in FIG. 17.

The equation for Aβ42/40 ratio PK/PD model is presented below:

$$\frac{dR}{dt} = {K}_{in} \cdot [ 1 + SLOPE \cdot Conc ] - R ( t ) \cdot {K}_{out}$$

Estimated parameters included baseline plasma Aβ42/40 ratio, indirect response parameters (Kᵢₙ and Kₒᵤₜ) and slope of drug effect (SLOPE). Emax function was also explored. Inter-individual variability was estimated for the baseline and SLOPE. Residual variability was modeled using proportional model.

This model was applied to pooled data from lecanemab treatment arms in Study 201 Core and OLE with placebo arm in Study 201 Core. Estimation of model parameters was performed using first order conditional estimation with interaction (FOCEI).

Covariate analysis was performed for the effect of age, weight, APOE4 carrier status, sex, AD diagnosis (MCI or mild AD), anti-drug antibodies (ADA) and neutralizing ADA (NAb) at subject level on baseline, Kₒᵤₜ and SLOPE and baseline Aβ42/40 ratio value on SLOPE. If the ADA sample was positive, an NAb assay was performed. In the categorical covariate analysis of NAb status at subject level, ADA(+)/NAb(+) was assigned as NAb positive, and ADA(+)/NAb(−) or samples with missing NAb category (NAb assay was not performed) were assigned as NAb negative. One subject (ID=195) had ADA(−) but NAb(+) result, and this subject was assigned as NAb positive.

### Relationship Between Plasma Aβ42/40 Ratio and Amyloid PET SUVr or Clinical Efficacy Endpoints

From the PK/PD model for plasma Aβ42/40 ratio developed in Section 5.4.1.1, Bayesian predictions of PD parameters for each subject were made, using the NONMEM POSTHOC function. These predicted parameters were used to generate individually predicted change from baseline (CFB) for plasma Aβ42/40 ratio profile, which was then incorporated into the datasets for relationships between CFB for plasma Aβ42/40 ratio and CFB for amyloid PET SUVr or CFB as clinical efficacy endpoints to be used in the subsequent analyses.

### Model for Relationship Between Plasma Aβ42/40 Ratio and Amyloid PET SUVr (Study 201 Core and OLE)

In the previous analysis, the relationship between serum lecanemab concentration and amyloid PET SUVr reduction time course was best described by an indirect response model. Instead of lecanemab concentration, CFB for plasma Aβ42/40 ratio was used as a predictor to describe relationship between the Aβ42/40 ratio and the amyloid PET SUVr reduction.

CFB for PET SUVr (CFB SUVr) was related to model-predicted CFB for plasma Aβ42/40 ratio (CFB ABETA) at the time of the assessment. The relationship between the CFB for Aβ42/40 ratio and CFB for PET SUVr was described by a direct response model as indicated in the following equation:

$$CFB = INT + ( {E}_{max} \cdot CFB ABETA ) / ( E 50 + CFB ABETA )$$

Estimated parameters included intercept (INT), maximum drug effect (Eₘₐₓ) and CFB for plasma Aβ42/40 ratio resulting in half of the maximum drug effect (E50). Linear function was also tested. Inter-individual variability as additive model was estimated for the intercept and Emax. Residual variability was modeled using additive model.

This model was applied to data from Study 201 Core and OLE. Estimation of model parameters was performed using FOCEI.

### Model for Relationship Between Plasma Aβ42/40 Ratio and Clinical Efficacy Endpoints (Study 201 Core)

In the previous analysis, the relationship between serum lecanemab concentration and clinical efficacy endpoints (CDR-SB, ADCOMS, and ADAS-cog) time course was best described by a PK/PD model with the exposure effect on disease progression as a linear function. Slowing of disease progression rate for CDR-SB, ADCOMS, and ADAS-Cog was positively correlated with lecanemab average steady-state concentration (Css,av). Each clinical efficacy endpoint (EFF) was expressed as an absolute score. An exposure-response model was developed introducing lecanemab exposure effect on the disease progression (DESLOPE) and baseline clinical score (INT) and disease progression rate (SLP) as below:

$$EFF = INT + SLP \cdot ( 1 - DESLOPE \cdot {C}_{ss , av} ) \cdot Time$$

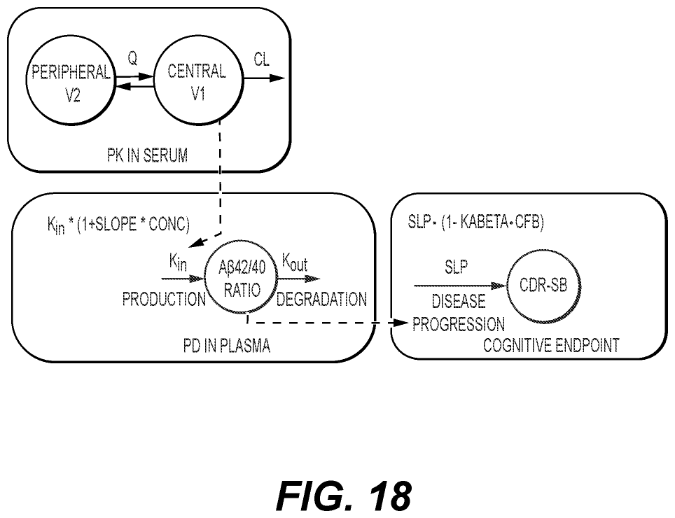

To explore the relationship between CFB for plasma Aβ42/40 ratio and clinical efficacy endpoints (CDR-SB, ADCOMS and ADAS-Cog), the model predicted CFB for plasma Aβ42/40 ratio was also evaluated as a predictor of the efficacy endpoints (referred to as the plasma Aβ42/40 ratio-Efficacy model), as indicated in FIG. 18.

Absolute clinical efficacy endpoint score (EFF) over time was related to model-predicted CFB for plasma Aβ42/40 ratio at the time of the assessment. The relationship between the CFB for Aβ42/40 ratio and clinical efficacy endpoint time course was described by a model that associates the greater change from baseline for plasma Aβ42/40 ratio with slower disease progression (SLP). The equation for plasma Aβ42/40 ratio-Efficacy is presented below:

$$EFF = INT + SLP \cdot ( 1 - KABETA \cdot CFB ABETA ) \cdot Time$$

Estimated parameters included baseline clinical score (INT), the effect of change from baseline for plasma Aβ42/40 ratio on the disease progression (KABETA) and disease progression rate (SLP). Inter-individual variability was estimated for baseline and SLP. Residual variability was modeled using proportional and additive model.

This model was applied to data from Study 201 Core (placebo and lecanemab treatment arms). Estimation of model parameters was performed using FOCEI.

For PK/PD analysis of plasma Aβ42/40 ratio, all subjects receiving lecanemab with serum PK information or receiving placebo in Study 201 Core and who had baseline and at least one post-dose Aβ42/40 ratio assessment were included. Subjects treated with lecanemab 10 mg/kg biweekly in the OLE and who had baseline and post-dose Aβ42/40 ratio assessment were also included.

For plasma Aβ42/40 ratio-amyloid PET SUVr modeling, subjects receiving lecanemab or placebo in Study 201 Core and OLE and who had baseline and at least one post-dose Aβ42/40 ratio assessment as well as baseline and at least one post-dose PET SUVr value were included. Subjects receiving lecanemab in Study 201 Core and who did not have Core baseline value of SUVr were excluded because CFB could not be calculated.

For plasma Aβ42/40 ratio-efficacy modeling, subjects receiving lecanemab or placebo in Study 201 Core and who had baseline and at least one post-dose Aβ42/40 ratio assessment baseline and at least one post-dose CDR-SB, ADCOMS and ADAS-cog value were included.

Population mean plasma Aβ42/40 ratio-time profiles were simulated for the following dosing regimens, based on the estimates from the final PK/PD model.
10 mg/kg biweekly for 42 months.
10 mg/kg biweekly for 18 months, followed by 24 months treatment discontinuation.
10 mg/kg biweekly for 18 months, followed by 10 mg/kg monthly for 24 months.
10 mg/kg biweekly for 18 months, followed by 10 mg/kg every 3 months for 24 months.

### PK/PD Model for Amyloid PET

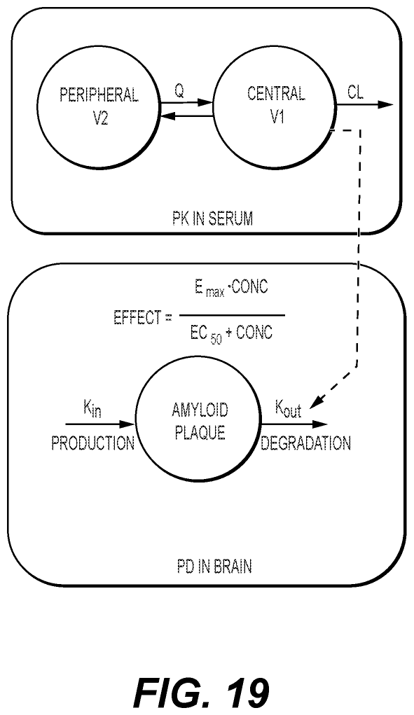

In the previous analysis using data from Study 201 Core, the relationship between serum lecanemab concentration and the amyloid SUVr reduction time course was best described by an indirect response model with serum lecanemab concentration in the central compartment inducing the reduction of amyloid plaque with degradation rate Kₒᵤₜ, as indicated in FIG. 19.

The equation for SUVr PK/PD model is presented below:

$$\frac{dSUVr}{dt} = {K}_{in} - SUVr ( t ) \cdot {K}_{out} \cdot [ 1 + \frac{{E}_{max} \cdot Conc}{{EC}_{50} + Conc} ]$$

Estimated parameters included baseline SUVr (BSUVr₀), indirect response parameters: Kᵢₙ is the production rate of amyloid plaque and Kₒᵤₜ is the rate constant of degradation of plaque, maximum drug effect (Eₘₐₓ) and lecanemab concentration resulting in half of the maximum drug effect (EC₅₀). In the Eₘₐₓ models, inter-individual variability (IIV) was estimated for baseline and Eₘₐₓ, while IIV could not be estimated for both Kin and EC50. Residual variability was modeled using proportional model. This PK-SUVr model was re-evaluated after adding data from subjects in Study 201 OLE. Estimation of model parameters was performed using FOCEI.

Covariate analysis was performed for the effect of age, weight, APOE4 carrier status, sex, AD diagnosis (MCI or mild AD), ADA and NAb status at subject level and baseline PET value (for Centiloid only). If the ADA sample was positive, a NAb assay was performed. In the categorical covariate analysis of NAb status at subject level, ADA(+)/NAb(+) was assigned as NAb positive, and ADA(+)/NAb(−) or samples with missing NAb category (NAb assay was not performed) were assigned as NAb negative. One subject (ID=195) had ADA(−) but NAb(+) result, and this subject was assigned as NAb positive.

The impact of covariates on the SUVr reduction was evaluated using a forest plot analysis. The SUVr change from baseline at 18 months with extreme values (5th and 95th percentile) of each individual covariate were simulated and compared with a reference SUVr change derived based on population typical covariate values. The median and 95% CIs of individual covariate effects were generated based on 1000 simulations using final estimates of typical values and the variance-covariance matrix from final PK/PD model.

The same analyses were conducted using Centiloid scale. Data from amyloid PET assessments by flutemetamol in Study 201 OLE were included in the PK/PD analysis using Centiloid scale. In addition, to avoid negative values in observed amyloid PET data in Centiloid scale, the modeling was performed using transformed value by adding 100 to the original value (ie, transformed value=original value+100). Residual variability was modeled using the additive model.

### Simulations

The dose-dependence in the lecanemab exposure-amyloid removal relationship was shown by comparing population mean SUVr (or Centiloid)-time profiles for 10 mg/kg biweekly and 10 mg/kg monthly over 18 months. In addition, to explore the impact of potential dose adjustments, population mean SUVr (or Centiloid)-time profiles were simulated for the following dosing scenarios:
10 mg/kg biweekly for 18 months for 42 months
10 mg/kg biweekly for 18 months, followed by 24 months treatment discontinuation
10 mg/kg biweekly for 18 months, followed by 10 mg/kg monthly for 24 months
10 mg/kg biweekly for 18 months, followed by 10 mg/kg every 3 months for 24 months
10 mg/kg biweekly for 18 months, followed by 10 mg/kg every 6 months for 24 months

The simulations accounted for the uncertainty of PD parameter estimates. Uncertainty was assessed by sampling 1000 sets of PD parameters, using final estimates of typical values and the variance-covariance matrix from final PK/PD models.

In addition, based on the 1000 model-predicted individual profiles considering inter-individual variabilities of PD parameters, the % of subjects achieving amyloid negativity for SUVr (<1.17) and PET Centiloid (<30.0) at 3, 6, 9, 12, 15 and 18 months following continuous treatment with lecanemab at 10 mg/kg biweekly, 10 mg/kg monthly, 10 mg/kg every 3 months and 10 mg/kg every 6 months were simulated. The quantitative threshold for amyloid negativity was defined as PET SUVr <1.17, corresponding to Centiloid cut off of 30 Centiloid (Fleisher, et al., 2011).

### Results:

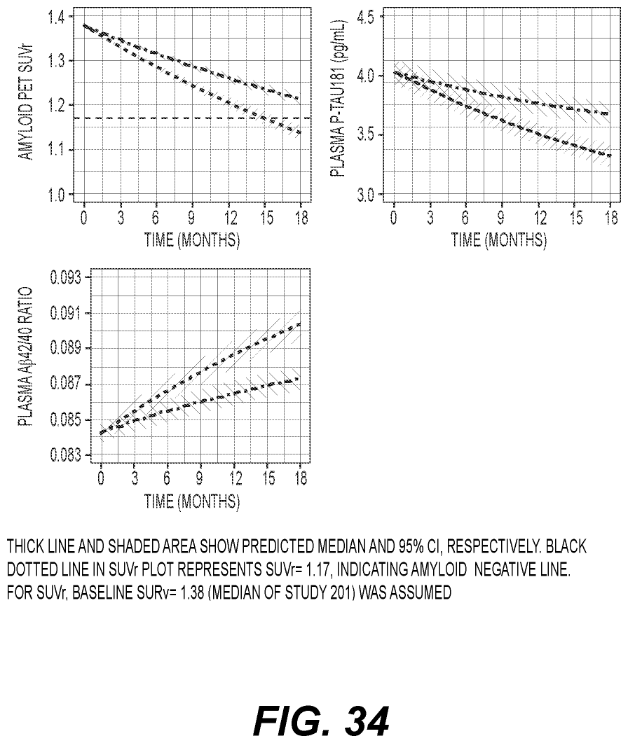

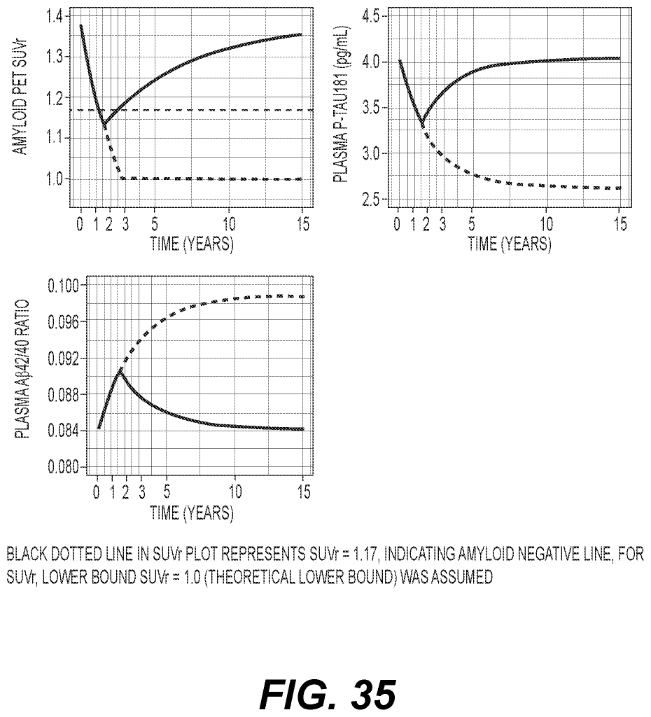

FIG. 34 summarizes the model-predicted, dose-dependent decrease in SUVr and p-tau181 and increase in Aβ42/40 ratio following 18 months treatment with lecanemab at 10 mg/kg biweekly or 10 mg/kg monthly.

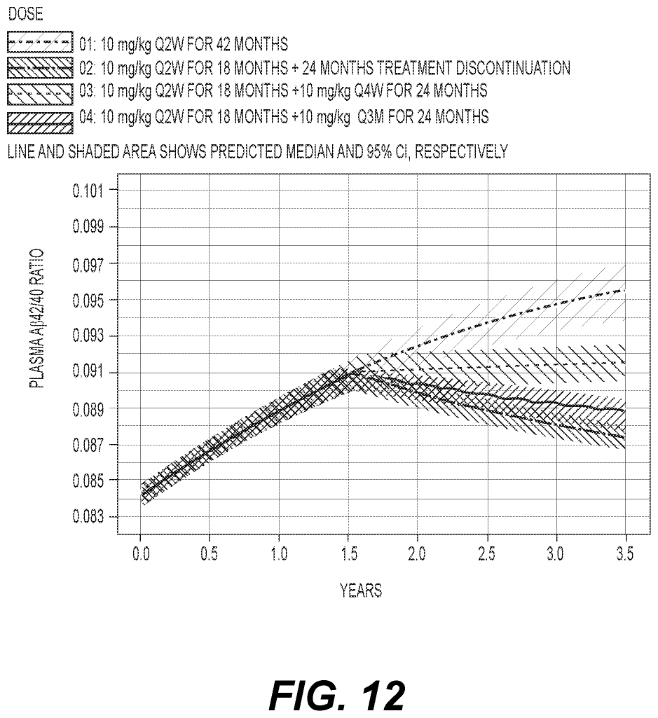

As depicted below in FIG. 12, when treatment at 10 mg/kg biweekly is continued beyond 18 months, plasma Aβ42/40 ratio is predicted to continue increasing further, whereas once treatment is discontinued after 18 months, plasma Aβ42/40 ratio is predicted to start to decrease slowly. A maintenance dose of 10 mg/kg monthly is predicted to maintain plasma Aβ42/40 ratio at a level achieved following 18 months of treatment at 10 mg/kg biweekly.

For alternative dosing regimens at 10 mg/kg biweekly with a monthly maintenance dose of 10 mg/kg for 18 months, plasma Aβ42/40 ratio is predicted to be maintained at a relatively constant level for a duration of 2 years of treatment. However, for a dosing regimen of 10 mg/kg biweekly for 18 months followed by a maintenance dose of 10 mg/kg every 3 months for 2 years, the plasma Aβ42/40 ratio starts to decrease gradually, slightly at a lower rate compared to that following treatment discontinuation after 18 months of treatment at 10 mg/kg.

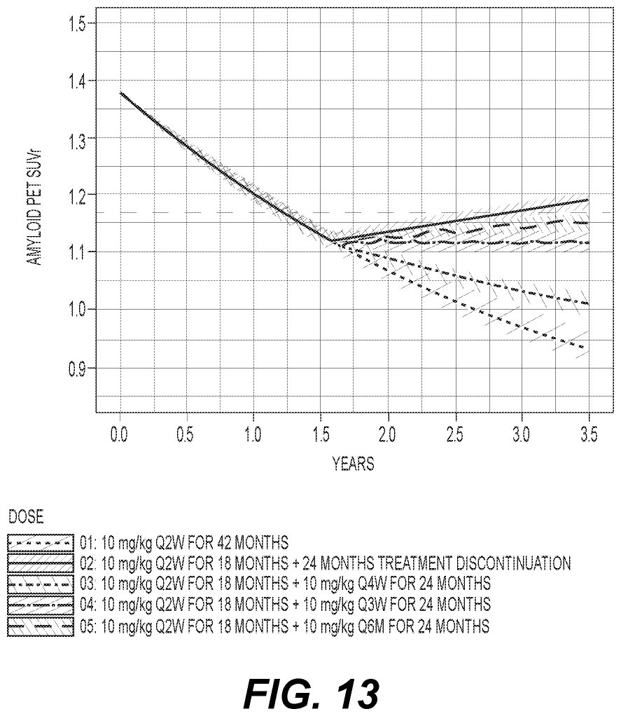

As depicted in FIG. 13, when treatment at 10 mg/kg biweekly is continued beyond 18 months, amyloid PET SUVr is predicted to continue decreasing further, whereas once treatment is discontinued after 18 months, amyloid PET SUVr is predicted to start to increasing slowly, an indication of slow re-accumulation of amyloid plaque. For alternative dosing regimens with less frequent dosing (monthly, every 3 months and every 6 months) after 10 mg/kg biweekly for 18 months, SUVr is predicted to be maintained at below the SUVr amyloid negativity of 1.17, as measured by florbetapir, for the full 24 months of simulated treatment. For a dosing regimen of 10 mg/kg biweekly for 18 months followed by either 10 mg/kg biweekly or 10 mg/kg monthly SUVr continues to decline further, however, with a slower rate of decline following 10 mg/kg monthly.

Modeling data in FIG. 35 illustrates that when treatment at 10 mg/kg biweekly is continued beyond 18 months, amyloid PET SUVr is predicted to continue declining to the lower bound of SUVr=1.0, whereas once treatment is discontinued after 18 months, amyloid PET SUVr is predicted to start increasing slowly and take over 15 years for amyloid to reaccumulate to baseline levels prior to treatment initiation with lecanemab. Plasma Aβ42/40 ratio and p-tau181 is predicted to take approximately 6-8 years for both biomarkers to reach a plateau by continuous lecanemab dosing at 10 mg/kg biweekly, or to return to baseline levels after the treatment discontinuation.

### Conclusions

The rate of change in plasma Aβ42/40 ratio relative to amyloid PET in the PK/PD modeling data suggests that the plasma Aβ42/40 ratio may be a more sensitive indicator of amyloid change and plaque accumulation compared to amyloid PET.

Clinical benefit of lecanemab treatment correlated with brain amyloid reduction (PET SUVr) and changes in plasma biomarkers of amyloid pathology (such as the Aβ42/40 ratio).

### PK/PD Analysis for Plasma Aβ42/40 Ratio

PK/PD analysis on data from 284 subjects in the Study 201 Core and OLE, indicated that longitudinal changes of plasma Aβ42/40 ratio were related to model-predicted lecanemab serum concentration at the time of the assessment by an indirect response model with the lecanemab concentration increasing the plasma Aβ42/40 ratio. Estimated model parameters included baseline plasma Aβ42/40 ratio, degradation rate constant of plasma Aβ42/40 ratio (Kₒᵤₜ) and slope of drug effect. The model describing exposure effect as Eₘₐₓ function had large condition number (>1000), therefore the model using a linear function for the exposure effect was selected. Based on the population estimate of Kₒᵤₜ, the half-life of plasma Aβ42/40 ratio degradation was estimated to be approximately 1.9 years. This suggests that the Aβ42/40 ratio would return to pretreatment levels after 4-5 half-lives, e.g., 8-10 years.

Covariate analysis was performed for the effect of age, weight, APOE4 carrier status, sex, AD diagnosis (MCI or mild AD), ADA and neutralizing ADA at subject level and baseline Aβ42/40 ratio value. However, none of these covariates were found to have a significant effect on either baseline, Kₒᵤₜ or slope.

Model-based simulations were used to explore the effects of dose on plasma Aβ42/40 ratio increase. The simulations showed that 18 months of treatment at 10 mg/kg biweekly resulted in higher lecanemab exposure, yielding a larger and faster increase in Aβ42/40 ratio with time than those following 10 mg/kg monthly for 18 months.

When treatment at 10 mg/kg biweekly is continued beyond 18 months, plasma Aβ42/40 ratio was predicted to increase further, whereas once treatment is discontinued after 18 months, plasma Aβ42/40 ratio starts to decrease slowly. Among the alternative dosing regimens, at 18-months 10 mg/kg biweekly treatment followed by less frequent monthly dosing, the plasma Aβ42/40 ratio was predicted to remain relatively constant for a duration of 2 years of simulated treatment. However, for a dosing regimen of 10 mg/kg biweekly for 18 months followed by 10 mg/kg every 3 months for 2 years, plasma Aβ42/40 ratio were predicted to start to decrease gradually, at a slightly lower rate compared to discontinuing lecanemab treatment after 18 months of treatment at 10 mg/kg.

Conclusions from the PK/PD Analysis for Aβ42/40 Ratio are:

The relationship between serum lecanemab concentration and plasma Aβ42/40 ratio time course in Study 201 Core and OLE was characterized by an indirect response model with lecanemab increasing the plasma Aβ42/40 ratio.

None of the covariates examined (age, weight, APOE4 carrier status, sex, AD diagnosis on baseline, Kₒᵤₜ and slope and of ADA, neutralizing ADA at subject level and baseline plasma Aβ42/40 ratio value on slope) were identified to be significant. However, conclusions from covariate analyses should be viewed with caution because of the small sample sizes of the dataset.

The half-life of plasma Aβ42/40 ratio degradation was estimated to be approximately 1.9 years, which is shorter than the half-life of PET SUVr (approximately 4 years) indicating that changes of plasma Aβ42/40 ratio is reflecting the early dynamic aggregation process of soluble Aβ aggregates compared to slow accumulation of Aβ fibrils to amyloid plaques.

Simulations showed that, following 18 months of treatment, 10 mg/kg biweekly dosing results in a larger and faster increase in plasma Aβ42/40 ratio with time compared to 10 mg/kg monthly dosing.

Simulations suggest that a maintenance dose of 10 mg/kg monthly is predicted to maintain plasma Aβ42/40 ratio at a level achieved following 18 months of treatment at 10 mg/kg biweekly.

### Exploratory Analysis of Relationship Between Plasma Aβ42/40 Ratio and Amyloid PET SUVr

For plasma Aβ42/40 ratio and amyloid PET SUVr modeling, 127 subjects receiving lecanemab or placebo in Study 201 Core and OLE and who had baseline and at least one post-dose Aβ42/40 ratio assessment as well as baseline and at least one post-dose PET SUVr value were included. Plasma Aβ42/40 ratio was a significant predictor of SUVr reduction. The relationship between the CFB of plasma Aβ42/40 ratio and CFB of SUVr was described by a direct Eₘₐₓ model with the CFB of plasma Aβ42/40 ratio increasing the CFB of SUVr.

Covariate analysis was performed for the effect of age, weight, APOE4 carrier status, sex, AD diagnosis (MCI or mild AD), ADA and neutralizing ADA at subject level and baseline Aβ42/40 ratio value. However, none of these covariates were found to have a significant effect on the relationship between plasma Aβ42/40 ratio and amyloid PET SUVr.

Conclusions from Exploratory Analysis of Relationship Between Plasma Aβ42/40 Ratio and Amyloid PET:

Plasma Aβ42/40 ratio was a significant predictor of SUVr reduction. The relationship between the CFB of plasma Aβ42/40 ratio and CFB of SUVr was described by a direct Eₘₐₓ model with the CFB of plasma Aβ42/40 ratio increasing the CFB of SUVr.

None of the covariates examined (age, weight, APOE4 carrier status, sex, AD diagnosis, ADA, neutralizing ADA at subject level and baseline plasma Aβ42/40 ratio value) were identified to be significant for the relationship between plasma Aβ42/40 ratio and amyloid PET SUVr. However, conclusions from covariate analyses should be viewed with caution because of the small sample sizes of the dataset.

### Exploratory Analyses of Relationship Between Plasma Aβ42/40 Ratio and Clinical Efficacy Endpoints

Relationships between the increase of the plasma Aβ42/40 ratio and key clinical endpoints (CDR-SB, ADCOMS and ADAS-Cog) were explored based on data from a subset of subjects (N=284) who had baseline and at least one post-dose Aβ42/40 ratio assessment in Study 201 Core. Relationships for change from baseline (CFB) of plasma Aβ42/40 ratio versus CFB of clinical endpoints over 18 months of treatment with different dosing regimen of lecanemab or placebo were subjected to nonlinear mixed effects modeling.

For CDR-SB and ADCOMS, increase in the plasma Aβ42/40 ratio from baseline over a treatment period of 18 months was a significant predictor of slowing of cognitive decline. Increase in plasma Aβ42/40 ratio from baseline was not a significant predictor of slowing of ADAS-Cog decline. Diagnosis of mild AD dementia had a significant effect on CDR-SB and ADCOMS baseline.

The plasma Aβ42/40 ratio-CDR-SB/ADCOMS models showed that a greater increase of plasma Aβ42/40 ratio was associated with a slower disease progression as measured by CDR-SB and ADCOMS. Model predicted disease progression rates in CDR-SB and ADCOMS were reduced by 6.76% and 5.97%, respectively, for every 0.002 unit increase from baseline in plasma Aβ42/40 ratio. The model predicted increase from baseline in plasma Aβ42/40 ratio over 18 months of 10 mg/kg biweekly was 0.0065; the model predicts corresponding reduction of progression rates in CDR-SB and ADCOMS of 22.0% and 19.4%.

Conclusions from Exploratory Analyses of Relationship Between Plasma Aβ42/40 Ratio and Clinical Efficacy Endpoints:

For CDR-SB and ADCOMS, an increase in plasma Aβ42/40 ratio from baseline over a treatment period of 18 months was a significant predictor of slowing cognitive decline. Although an estimated slope suggested that the trend of plasma Aβ42/40 ratio change dependent slowing of disease progression of ADAS-Cog, an increase in plasma Aβ42/40 ratio from baseline was not a significant predictor of slowing of ADAS-Cog decline, possibly due to high variability in ADAS-Cog in the dataset from the 284 subjects.

For both CDR-SB and ADCOMS there were no clinically significant covariate effects.

The plasma Aβ42/40 ratio-CDR-SB/ADCOMS models showed that a greater increase of plasma Aβ42/40 ratio was associated with a slower rate of cognitive decline, measured as a slower increase in CDR-SB and ADCOMS.

Model predicted disease progression rates in CDR-SB and ADCOMS were reduced by 6.76% and 5.97%, respectively, for every 0.002 unit increase from baseline in plasma Aβ42/40 ratio.

For lecanemab 10 mg/kg biweekly, the model predicted increase from baseline in plasma Aβ42/40 ratio over 18 months was 0.0065; the corresponding model predicted reduction of disease progression rates in CDR-SB and ADCOMS were 22.0% and 19.4%, respectively.

Conclusions from the PK/PD Analysis for Amyloid PET SUVr:

The relationship between serum lecanemab concentration and the SUVr reduction time course in Study 201 Core and OLE was characterized by an indirect response model with lecanemab inducing the reduction of amyloid plaque.

Covariates identified in the final PK/PD model for SUVr indicated that APOE4 carriers have higher baseline SUVr, and older subjects have higher maximum plaque removal (Emax).

The final PK/PD model indicates subjects with higher baseline SUVr have greater overall SUVr reductions.

ADA status and neutralizing ADA status at subject level did not impact drug effect on SUVr.

The half-life of amyloid re-accumulation is estimated to be approximately 4 years (0.693/Kout), suggesting that it will take approximately 16-20 years (based on 4-5 half-lives) for amyloid to re-accumulate and return to its value prior to treatment initiation with lecanemab.

Simulations showed that 18 months of treatment at 10 mg/kg biweekly dosing results in a larger and faster decrease in SUVr over time compared to 10 mg/kg monthly dosing.

The percent of subjects achieving amyloid negativity for SUVr at 3, 6, 9, 12, 15 and 18 months following 10 mg/kg biweekly is higher than corresponding values following 10 mg/kg monthly.

Simulations suggest that a dosing regimen at 10 mg/kg biweekly for 18 months followed by a maintenance dose of 10 mg/kg every 3 months is predicted to maintain SUVr constant and below an amyloid PET SUVr negativity (e.g. for florbetapir, PET SUVr of 1.17).

### Abbreviation Used in the Examples

Aβ amyloid beta
Aβ(1-42) amyloid beta monomer from amino acid 1 to 42
AD Alzheimer's disease
ADAS-Cog Alzheimer's Disease Assessment Scale-Cognitive Subscale
ADCOMS Alzheimer's Disease Composite Score
ADNI Alzheimer's Disease Neuroimaging Initiative
APOE Apolipoprotein E
APOE4 apolipoprotein ε4 variant
ARIA amyloid-related imaging abnormalities
ARIA-E amyloid related imaging abnormality edema/effusion
ARIA-H amyloid related imaging abnormality hemorrhage
BAN2401 a humanized IgG1 monoclonal antibody
CDR Clinical Dementia Rating
CDR-SB Clinical Dementia Rating-Sum of Boxes
COVID-19 Coronavirus Disease 2019
CSF cerebrospinal fluid
EAD early Alzheimer's Disease
FAQ Functional Assessment Questionnaire
IV intravenous
LC-MS/MS liquid chromatography-tandem mass spectrometry
MCI mild cognitive impairment
MMSE Mini Mental State Examination
MRI magnetic resonance imaging
NIA-AA National Institute of Aging-Alzheimer's Association
OLE open-label Extension
PET positron emission tomography
p-tau phospho-tau
RAR response adaptive randomization
SUVR standard uptake value ratio
t½ terminal elimination half-life
t-tau total tau
vMRI volumetric magnetic resonance imaging

1-49. (canceled)

50. A method of treating Alzheimer's disease (AD) in a subject in a subject having, suspected of having, or at risk for developing Alzheimer's disease (AD), comprising:
a. measuring a concentration of amyloid β 1-42 (Aβ42) and a concentration of amyloid β 1-40 (Aβ40) in a blood sample obtained from the subject to determine a ratio of Aβ42 to Aβ40 (Aβ42/40 ratio); and
b. administering a treatment comprising a therapeutically effective dose of an anti-amyloid β (Aβ) protofibril antibody to the subject having an Aβ42/40 ratio that indicates amyloid positive status,
wherein the anti-Aβ protofibril antibody comprises a heavy chain variable region comprising an amino acid sequence of SEQ ID NO: 7 and a light chain variable region comprising an amino acid sequence of SEQ ID NO: 8.

51. The method of claim 50, further comprising, after administering the treatment:
c. measuring a second concentration of Aβ42 and a second concentration of Aβ40 in a blood sample obtained from the subject to determine a second Aβ42/Aβ40 ratio; and
d. administering a second treatment comprising a therapeutically effective dose of the anti-Aβ protofibril antibody to the subject having an Aβ42/40 ratio that indicates amyloid positive status.

52. The method of claim 50, further comprising, after administering the treatment:
c. measuring a second concentration of Aβ42 and a second concentration of Aβ40 in a blood sample obtained from the subject to determine a second Aβ42/Aβ40 ratio; and
d. administering a second treatment comprising a maintenance dose of the anti-Aβ protofibril antibody to the subject having an Aβ42/40 ratio that indicates amyloid negative status.

53. The method of claim 52, comprising administering further maintenance doses of the anti-Aβ protofibril antibody to the subject according to a maintenance dosing regimen, wherein the subject has an Aβ42/40 ratio that indicates amyloid negative status.

54. The method of claim 50, wherein the Aβ42/40 ratio that indicates amyloid positive status is at most 0.1.

55. The method of claim 50, wherein the Aβ42/40 ratio that indicates amyloid positive status is a ratio below about 0.092 to 0.094.

56. The method of claim 55, wherein the Aβ42/40 ratio that indicates amyloid positive status is a ratio below 0.092.

57. The method of claim 50, wherein the subject has Alzheimer's disease.

58. The method of claim 50, wherein the subject has early Alzheimer's disease.

59. The method of claim 50, wherein the subject has pre-Alzheimer's disease (pre-AD).

60. The method of claim 50, wherein the subject is cognitively normal but exhibits at least one biomarker of AD.

61. The method of claim 50, wherein the subject has been diagnosed with
a. mild cognitive impairment due to Alzheimer's disease-intermediate likelihood and/or has been diagnosed as having mild Alzheimer's disease dementia;
b. mild cognitive impairment due to Alzheimer's disease-intermediate likelihood by National Institute of Aging-Alzheimer's Association (NIA-AA) core clinical criteria;
c. mild cognitive impairment due to Alzheimer's disease-intermediate likelihood by a CDR global score of 0.5 and a Memory Box score of 0.5 or greater before treatment;
d. mild cognitive impairment due to Alzheimer's disease-intermediate likelihood by a history of subjective memory decline with gradual onset and slow progression over the last 1 year before treatment as corroborated by an informant;
e. mild Alzheimer's disease dementia by the NIA-AA core clinical criteria for probable Alzheimer's disease dementia; or
f. mild Alzheimer's disease dementia by a CDR score of 0.5 to 1.0 and a Memory Box score of 0.5 or greater before treatment.

62. The method of claim 50, wherein the amyloid-positive status is confirmed by a PET assessment, a CSF assessment of Aβ(1-42), MRI, retinal amyloid accumulation, and/or specific behavioral/cognitive phenotypes.

63. The method of claim 50, wherein the subject has at least one copy of the ApoE4 gene.

64. The method of claim 50, wherein the concentrations of Aβ42 and Aβ40 used to obtain the Aβ42/40 ratio are measured using an LC MS/MS assay.

65. The method of claim 52, wherein an elevated Aβ42/40 ratio after treatment with the anti-protofibril antibody indicates that the treatment has converted the subject from amyloid-positive to amyloid-negative status.

66. The method of claim 52, wherein the Aβ42/40 ratio after treatment with the anti-protofibril antibody is above 0.092.

67. The method of claim 50, wherein the treatment is discontinued if, after treatment with the therapeutically effective dose of the anti-protofibril antibody, further measurements of Aβ42 and Aβ40 concentration in a blood sample obtained from the subject result in an Aβ42/Aβ40 ratio that does not indicate a conversion to amyloid negative status.

68. The method of claim 50, wherein the treatment comprises an intravenous administration of the anti-Aβ protofibril antibody at a therapeutically effective dose of 10 mg/kg relative to the weight of the subject.

69. The method of claim 68, wherein the therapeutically effective dose is administered every 2 weeks.

70. The method of claim 50, wherein the treatment comprises a subcutaneous administration of a therapeutically effective dose of the anti-Aβ protofibril antibody.

71. The method of claim 70, wherein the therapeutically effective dose of the anti-Aβ protofibril antibody is 720 mg.

72. The method of claim 70, wherein the therapeutically effective dose is administered weekly.

73. The method of claim 50, wherein the frequency of administration is reduced after 18 months or 24 months of treatment.

74. The method of claim 50, wherein the dosage of the anti-Aβ protofibril antibody is reduced after 18 months or 24 months of treatment.

75. The method of claim 50, wherein the subject is switched to a maintenance dosing regimen after 18 months or 24 months of treatment.

76. The method of claim 75, wherein the subject is switched to a maintenance dosing regimen when a further Aβ42/40 ratio obtained after treatment indicates amyloid negative status.

77. The method of claim 75, wherein the maintenance dosing regimen comprises an intravenous infusion of the anti-Aβ protofibril antibody at a therapeutically effective dose of 10 mg/kg relative to the weight of the subject, administered monthly.

78. The method of claim 75, wherein the maintenance dosing regimen comprises subcutaneous administration of the anti-Aβ protofibril antibody at a therapeutically effective dose of 360 mg, administered weekly.

79. The method of claim 70, wherein the subject is switched to a maintenance dosing regimen comprising administration of a maintenance dose of the anti-Aβ protofibril antibody that is 50% of the subcutaneous administration of the anti-Aβ protofibril antibody.

80. The method of claim 75, comprising detecting, before switching to a maintenance dose,
a. a decrease in p-tau181 in a blood sample from the subject, or
b. a brain amyloid negativity as measured by PET SUVr in the subject.

81. The method of claim 80, wherein the brain amyloid negativity measured by PET SUVr is a florbetapir PET SUVr level at or below 1.17.

82. The method of claim 75, wherein the maintenance dose is administered at a dose and/or frequency selected to maintain
a. an Aβ42/40 ratio in a blood sample from the patient that indicates amyloid negative status; and/or
b. a florbetapir PET SUVr level at or below 1.17.

83. The method of claim 50, wherein the subject is sequentially or simultaneously administered at least one additional AD medication.

84. The method of claim 83, wherein the additional AD medication is E2814.

85. The method of claim 50, wherein the method results in:
a. a reduction or a slowing of increase of one or more cerebrospinal fluid biomarkers selected from Aβ1-42, total tau, phosphorylated tau, neurogranin, and neurofilament light peptide; and/or
b. a reduction or a slowing of increase of plasma or serum biomarkers selected from total tau, phosphorylated tau, glial fibrillary acidic protein (GFAP), and neurofilament light (NfL);
as compared to the biomarker levels before treatment.

86. The method of claim 50, wherein the treatment
a. delays clinical decline as determined by ADCOMS;
b. delays clinical decline as determined by ADAS MCI-ADL;
c. delays clinical decline as determined by modified iADRS;
d. delays clinical decline as measured by a CDR-SB; or
e. delays clinical decline as measured by an ADAS-Cog.

87. The method of claim 50, further comprising monitoring for ARIA-E and/or ARIA-H by MRI.

88. The method of claim 50, wherein the use does not require a titration step prior to administering to the subject a first therapeutically effective dose of the anti-Aβ protofibril antibody.

89. The method of claim 50, wherein the subject has intermediate brain amyloid as measured by PET SUVr prior to treatment with the anti-Aβ protofibril antibody.

90. The method of claim 50, wherein the anti-Aβ protofibril antibody comprises a heavy chain comprising an amino acid sequence of SEQ ID NO: 9 and a light chain comprising an amino acid sequence of SEQ ID NO: 10.

91. A method of treating Alzheimer's disease (AD) in a subject, comprising:
a. measuring a concentration of amyloid β 1-42 (Aβ42) and a concentration of amyloid β 1-40 (Aβ40) in a blood sample obtained from a subject previously administered an anti-amyloid β (Aβ) protofibril antibody to determine a ratio of Aβ42 to Aβ40 (Aβ42/40 ratio); and
b. administering a maintenance dosing regimen comprising a therapeutically effective dose of the anti-Aβ protofibril antibody to the subject having an Aβ42/40 ratio that indicates amyloid negative status,
wherein the anti-Aβ protofibril antibody comprises a heavy chain variable region comprising an amino acid sequence of SEQ ID NO: 7 and a light chain variable region comprising an amino acid sequence of SEQ ID NO: 8.
